(19) United States
(12) Patent Application Publication
(10) Pub. No.: US 2017/0290920 A1
(43) Pub. Date: Oct. 12, 2017

(54) ANTI-CD22 ANTIBODIES AND IMMUNOCONJUGATES

(71) Applicants: MEDIMMUNE LIMITED, Cambridge (GB); GENENTECH, INC., South San Francisco, CA (US)

(72) Inventors: Paul Polakis, South San Francisco, CA (US); Andrew Polson, South San Francisco, CA (US); Susan Diane Spencer, South San Francsico, CA (US); Shang-Fan Yu, South San Francsico, CA (US); John A. Flygare, South San Francisco, CA (US); Janet L. Gunzner-Toste, South San Francisco, CA (US); Thomas H. Pillow, South San Francisco, CA (US); Philip Wilson Howard, London (GB); Luke Masterson, London (GB)

(73) Assignees: MEDIMMUNE LIMITED, CAMBRIDGE (GB); GENENTECH, INC., South San Francisco, CA (US)

(21) Appl. No.: 15/497,656

(22) Filed: Apr. 26, 2017

Related U.S. Application Data
(63) Continuation of application No. 13/936,279, filed on Jul. 8, 2013, now abandoned.
(60) Provisional application No. 61/669,272, filed on Jul. 9, 2012.
(60) Provisional application No. 61/777,113, filed on Mar. 12, 2013.

(51) Int. Cl. A61K 47/48; A61K 31/5517; C07D 487/16; C07K 19/00

(52) U.S. Cl. CPC A61K 47/4863; A61K 47/48561; A61K 31/5517; A61K 47/48669

(57) ABSTRACT

The invention provides anti-CD22 antibodies and immunoconjugates and methods of using the same.

This application is a continuation application of U.S. application Ser. No. 13/936,279, filed Jul. 8, 2013, which claims the benefit of U.S. Provisional Application No. 61/669,272, filed Jul. 9, 2012; and U.S. Provisional Application No. 61/777,113, filed Mar. 12, 2013; each of which is incorporated by reference herein in its entirety for any purpose.

### FIELD OF THE INVENTION

The present invention relates to immunoconjugates comprising anti-CD22 antibodies and methods of using the same.

### BACKGROUND

B-cell antigens, such as CD19, CD22, and CD52, represent targets of therapeutic potential for treatment of lymphoma (Grillo-Lopez A. J. et al., Curr Pharm Biotechnol, 2:301-11, (2001)). CD22 is a 135-kDa B-cell-restricted sialoglycoprotein expressed on the B-cell surface only at the mature stages of differentiation (Dorken, B. et al., J. Immunol. 136:4470-4479 (1986)). The predominant form of CD22 in humans is CD22beta which contains seven immunoglobulin superfamily domains in the extracellular domain (Wilson, G. L. et al., J. Exp. Med. 173:137-146 (1991)). A variant form, CD22 alpha, lacks immunoglobulin superfamily domains 3 and 4 (Stamenkovic, I. and Seed, B., Nature 345:74-77 (1990)). Ligand-binding to human CD22 has been shown to be associated with immunoglobulin superfamily domains 1 and 2 (also referred to as epitopes 1 and 2) (Engel, P. et al., J. Exp. Med. 181:1581-1586, 1995).

B cell-related disorders include, but are not limited to, malignant lymphoma (Non-Hodgkin's Lymphoma, NHL), multiple myeloma, and chronic lymphocytic leukemia (CLL, B cell leukemia (CD5+ B lymphocytes). Non-Hodgkin's lymphomas (NHLs), a heterogeneous group of cancers principally arising from B lymphocytes, represent approximately 4% of all newly diagnosed cancers (Jemal, A. et al., CA-Cancer J Clin, 52: 23-47, (2002)). Aggressive NHL comprises approximately 30-40% of adult NHL (Harris, N. L. et al., Hematol. J. 1:53-66 (2001)) and includes diffuse large B-cell lymphoma (DLBCL), mantle cell lymphoma (MCL), peripheral T-cell lymphoma, and anaplastic large cell lymphoma. Frontline combination chemotherapy cures less than half of the patients with aggressive NHL, and most patients eventually succumb to their disease (Fisher, R. I. Semin. Oncol. 27(suppl 12): 2-8 (2000)).

In B-cell NHL, CD22 expression ranges from 91% to 99% in the aggressive and indolent populations, respectively (Cesano, A. et al., Blood 100:350a (2002)). CD22 may function both as a component of the B-cell activation complex (Sato, S. et al., Semin. Immunol. 10:287-296 (1998)) and as an adhesion molecule (Engel, Pl t al., J. Immunol. 150:4719-4732 (1993)). The B cells of CD22-deficient mice have a shorter life span and enhanced apoptosis, which suggests a role of this antigen in B-cell survival (Otipoby, K. L. et al., Nature (Lond) 384:634-637 (1996)). After binding with its natural ligand(s) or antibodies, CD22 is rapidly internalized, providing a costimulatory signal in primary B cells and proapoptotic signals in neoplastic B cells (Sato, S. et al., Immunity 5:551-562 (1996)).

There is a need in the art for agents that target CD22 for the diagnosis and treatment of CD22-associated conditions, such as cancer. The invention fulfills that need and provides other benefits.

### SUMMARY

The invention provides anti-CD22 antibodies and immunoconjugates and methods of using the same.

In some embodiments, an immunoconjugate comprising an antibody that binds CD22 covalently attached to a cytotoxic agent is provided, wherein the antibody binds an epitope within amino acids 20 to 240 of SEQ ID NO: 28. In some embodiments, the cytotoxic agent is a pyrrolobenzodiazepine.

In some embodiments, the antibody comprises (i) HVR-H3 comprising the amino acid sequence of SEQ ID NO: 11, (ii) HVR-L3 comprising the amino acid sequence of SEQ ID NO: 14, and (iii) HVR-H2 comprising the amino acid sequence of SEQ ID NO: 10. In some embodiments, the antibody comprises (i) HVR-H1 comprising the amino acid sequence of SEQ ID NO: 9, (ii) HVR-H2 comprising the amino acid sequence of SEQ ID NO: 10, and (iii) HVR-H3 comprising the amino acid sequence of SEQ ID NO: 11. In some embodiments, the antibody comprises: a) (i) HVR-H1 comprising the amino acid sequence of SEQ ID NO: 9, (ii) HVR-H2 comprising the amino acid sequence of SEQ ID NO: 10, (iii) HVR-H3 comprising the amino acid sequence of SEQ ID NO: 11, (iv) HVR-L1 comprising an amino acid sequence selected from SEQ ID NOs: 12 and 15 to 22, (v) HVR-L2 comprising the amino acid sequence of SEQ ID NO: 13, and (vi) HVR-L3 comprising the amino acid sequence of SEQ ID NO: 14; or b) (i) HVR-H1 comprising the amino acid sequence of SEQ ID NO: 9, (ii) HVR-H2 comprising the amino acid sequence of SEQ ID NO: 10, (iii) HVR-H3 comprising the amino acid sequence of SEQ ID NO: 11, (iv) HVR-L1 comprising the amino acid sequence of SEQ ID NO: 15, (v) HVR-L2 comprising the amino acid sequence of SEQ ID NO: 13, and (vi) HVR-L3 comprising the amino acid sequence of SEQ ID NO: 14. In some embodiments, the antibody comprises: a) (i) HVR-L1 comprising an amino acid sequence selected from SEQ ID NOs: 12 and 15 to 22, (ii) HVR-L2 comprising the amino acid sequence of SEQ ID NO: 13, and (iii) HVR-L3 comprising the amino acid sequence of SEQ ID NO: 14; or b) (i) HVR-L1 comprising the amino acid sequence of SEQ ID NO: 15, (ii) HVR-L2 comprising the amino acid sequence of SEQ ID NO: 13, and (iii) HVR-L3 comprising the amino acid sequence of SEQ ID NO: 14. In some embodiments, the antibody comprises: a) a VH sequence having at least 95% sequence identity to the amino acid sequence of SEQ ID NO: 7; or b) a VL sequence having at least 95% sequence identity to the amino acid sequence of SEQ ID NO: 8; or c) a VH sequence as in (a) and a VL sequence as in (b). In some embodiments, the antibody comprises a VH sequence having the amino acid sequence of SEQ ID NO: 7. In some embodiments, the antibody comprises a VL sequence having the amino acid sequence of SEQ ID NO: 6 or a VL sequence having the amino acid sequence of SEQ ID NO: 8. In some embodiments, the antibody is an IgG1, IgG2a or IgG2b antibody.

In some embodiments, an immunoconjugate comprising an antibody that binds CD22 covalently attached to a cytotoxic agent is provided, wherein the antibody comprises (a) a VH sequence having the amino acid sequence of SEQ ID NO: 7 and a VL sequence having the amino acid sequence of SEQ ID NO: 8, and wherein the cytotoxic agent is a pyrrolobenzodiazepine.

In some embodiments, the immunoconjugate has the formula Ab-(L-D)p, wherein: (a) Ab is the antibody; (b) L is a linker; (c) D is the cytotoxic agent; and (d) p ranges from 1-8. In some such embodiments, D is a pyrrolobenzodiazepine of Formula A:

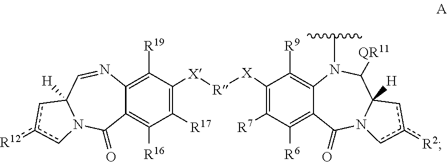

wherein the dotted lines indicate the optional presence of a double bond between C1 and C2 or C2 and C3;
R² is independently selected from H, OH, ═O, ═CH₂, CN, R, OR, ═CH—R^{D}, ═C(R^{D})₂, O—SO₂—R, CO₂R and COR, and optionally further selected from halo or dihalo, wherein R^{D} is independently selected from R, CO₂R, COR, CHO, CO₂H, and halo;
R⁶ and R⁹ are independently selected from H, R, OH, OR, SH, SR, NH₂, NHR, NRR′, NO₂, Me₃Sn and halo;
R⁷ is independently selected from H, R, OH, OR, SH, SR, NH₂, NHR, NRR′, NO₂, Me₃Sn and halo;
Q is independently selected from O, S and NH;
R¹¹ is either H, or R or, where Q is O, SO₃M, where M is a metal cation;
R and R′ are each independently selected from optionally substituted C₁₋₈ alkyl, C₃₋₈ heterocyclyl and C₅₋₂₀ aryl groups, and optionally in relation to the group NRR′, R and R′ together with the nitrogen atom to which they are attached form an optionally substituted 4-, 5-, 6- or 7-membered heterocyclic ring;
R¹², R¹⁶, R¹⁹ and R¹⁷ are as defined for R², R⁶, R⁹ and R⁷ respectively;
R″ is a C₃₋₁₂ alkylene group, which chain may be interrupted by one or more heteroatoms and/or aromatic rings that are optionally substituted; and
X and X′ are independently selected from O, S and N(H).

In some embodiments, D has the structure:

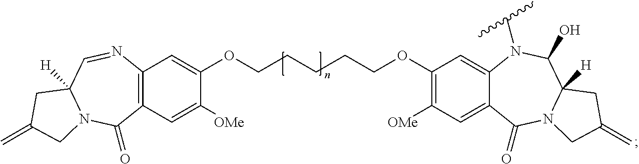

wherein n is 0 or 1.

In some embodiments, D has a structure selected from:

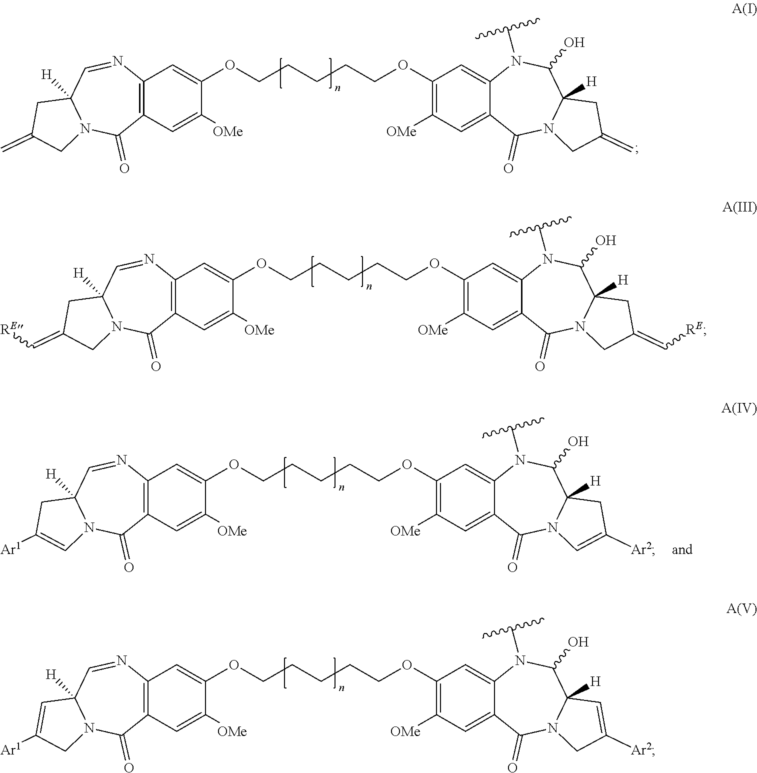

wherein R^{E} and R^{E″} are each independently selected from H or R^{D}, wherein R^{D} is independently selected from R, CO₂R, COR, CHO, CO₂H, and halo;
wherein Ar¹ and Ar² are each independently optionally substituted C₅₋₂₀ aryl; and wherein n is 0 or 1.

In some embodiments, D is a pyrrolobenzodiazepine of Formula B:

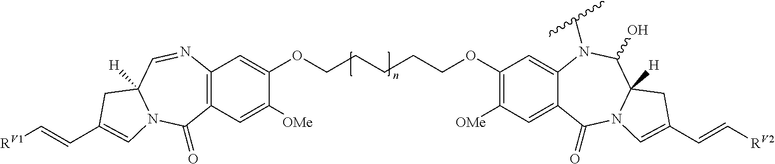

wherein the horizontal wavy line indicates the covalent attachment site to the linker; R^{V1} and R^{V2} are independently selected from H, methyl, ethyl, phenyl, fluoro-substituted phenyl, and C₅₋₆ heterocyclyl; and
n is 0 or 1.

In some embodiments, the immunoconjugate comprises a linker that is cleavable by a protease. In some such embodiments, the linker comprises a val-cit dipeptide or a Phe-homoLys dipeptide. In some embodiments, the immunoconjuge has the formula:

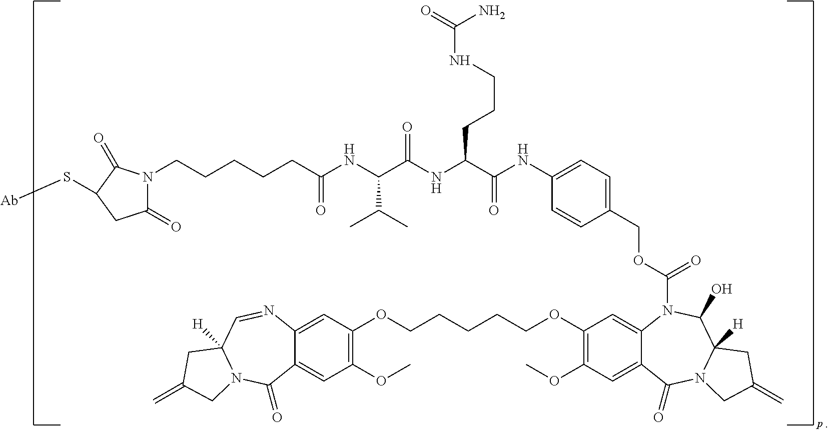

In some embodiments, p ranges from 1-3.

In some embodiments, the immunoconjugate comprises the structure:

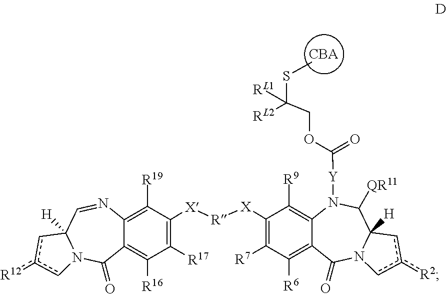

wherein CBA represents the antibody (Ab). In some embodiments, R^{L1} and R^{L2} are each independently selected from H and methyl, or together with the carbon atom to which they are bound form a cyclopropylene group. In some embodiments, Y is selected from a single bond, (a1), and (a2):

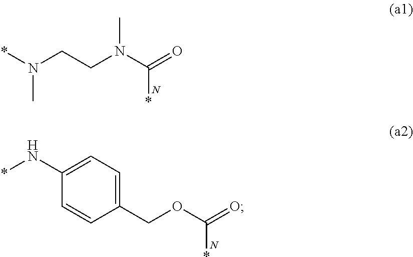

wherein N shows where the group binds to the N10 of the PBD moiety.

In some embodiments, the immunoconjugate comprises a structure selected from:

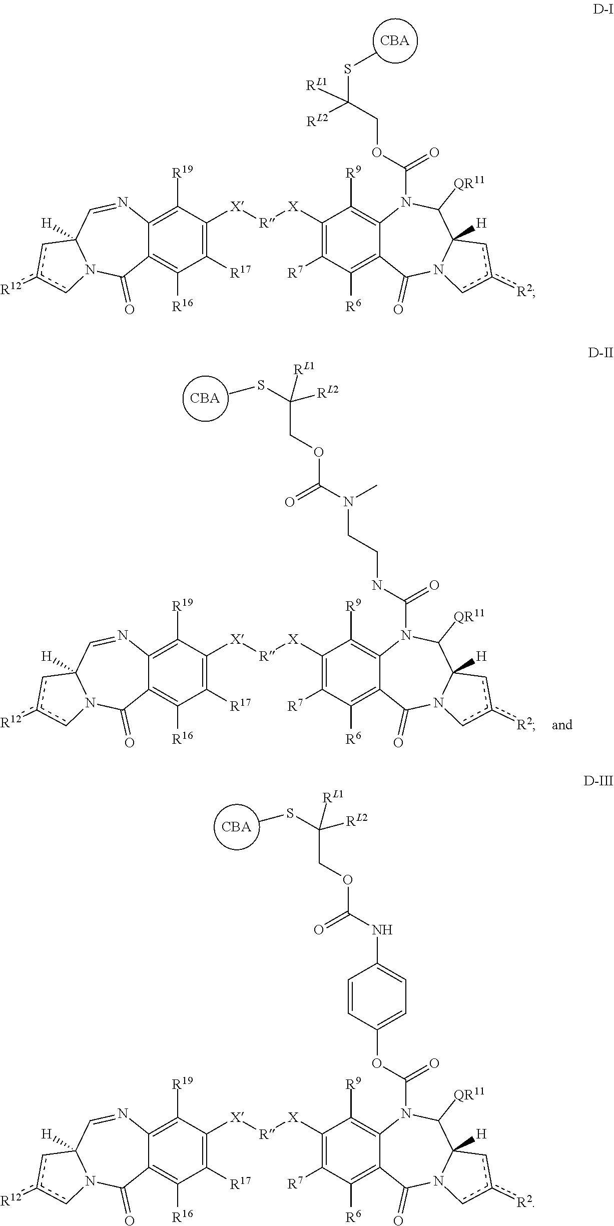

In some embodiments, the immunoconjugate comprises the structure:

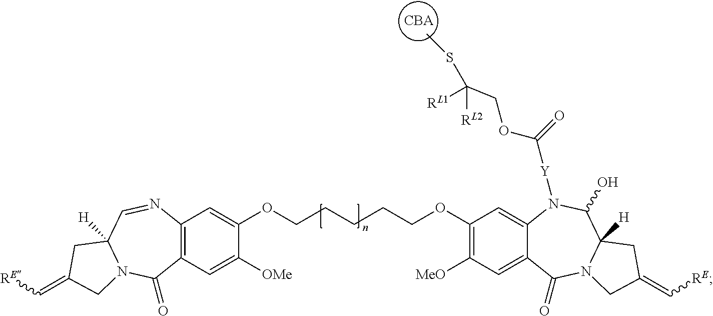

wherein R^{E} and R^{E″} are each independently selected from H and R^{D}.

In some embodiments, the immunoconjugate comprises the structure:

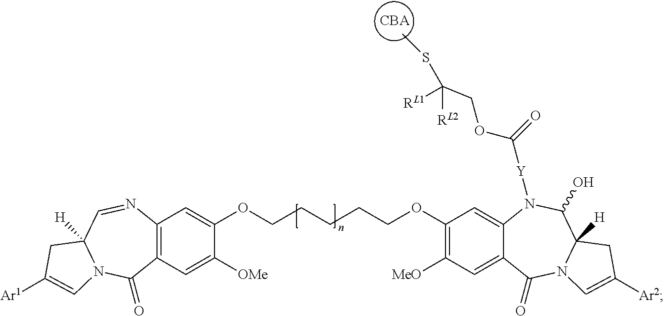

wherein Ar¹ and Ar² are each independently optionally substituted C₅₋₂₀ aryl. In some embodiments, Ar¹ and Ar² are each independently selected from optionally substituted phenyl, furanyl, thiophenyl and pyridyl.

In some embodiments, the immunoconjugate comprises the structure:

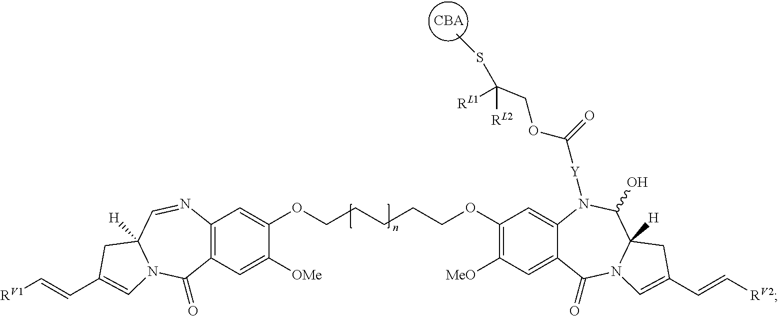

wherein R^{V1} and R^{V2} are each independently selected from H, methyl, ethyl, optionally substituted phenyl, and C₅₋₆ heterocyclyl. In some embodiments, R^{V1} and R^{V2} are each independently selected from H, phenyl, and 4-fluorophenyl.

In some embodiments, an immunoconjugate is provided that has a formula selected from:

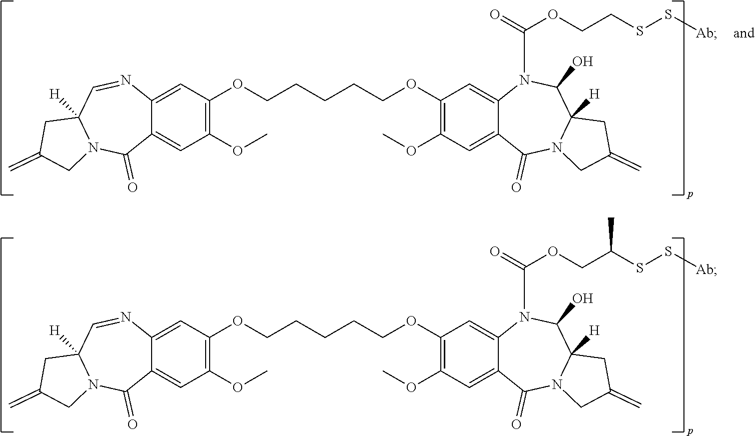

wherein Ab is an antibody comprising (i) HVR-H1 comprising the amino acid sequence of SEQ ID NO: 9, (ii) HVR-H2 comprising the amino acid sequence of SEQ ID NO: 10, (iii) HVR-H3 comprising the amino acid sequence of SEQ ID NO: 11, (iv) HVR-L1 comprising the amino acid sequence of SEQ ID NO: 15, (v) HVR-L2 comprising the amino acid sequence of SEQ ID NO: 13, and (vi) HVR-L3 comprising the amino acid sequence of SEQ ID NO: 14; and wherein p ranges from 1 to 3.

In some embodiments, an immunoconjugate is provided, wherein the immunoconjugate has the formula:

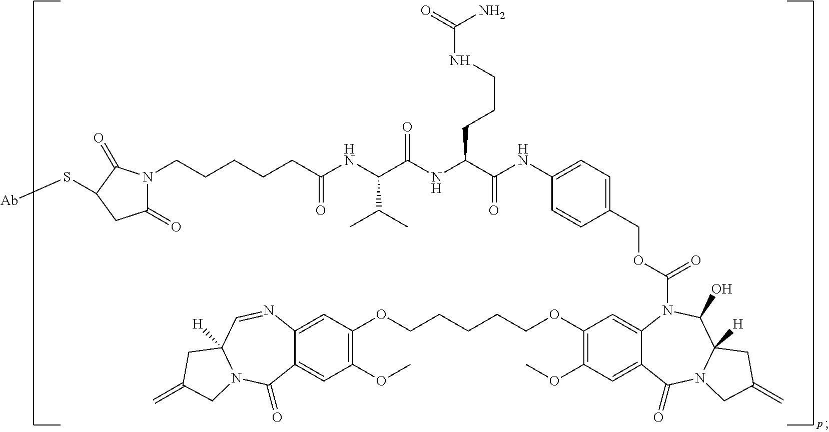

wherein Ab is an antibody comprising (i) HVR-H1 comprising the amino acid sequence of SEQ ID NO: 9, (ii) HVR-H2 comprising the amino acid sequence of SEQ ID NO: 10, (iii) HVR-H3 comprising the amino acid sequence of SEQ ID NO: 11, (iv) HVR-L1 comprising the amino acid sequence of SEQ ID NO: 15, (v) HVR-L2 comprising the amino acid sequence of SEQ ID NO: 13, and (vi) HVR-L3 comprising the amino acid sequence of SEQ ID NO: 14; and wherein p ranges from 1 to 3. In some such embodiments, the antibody comprises a VH sequence of SEQ ID NO: 7 and a VL sequence of SEQ ID NO: 8. In some embodiments, the antibody comprises a heavy chain of SEQ ID NO: 26 and a light chain of SEQ ID NO: 23.

In any of the embodiments discussed herein, the antibody may be a monoclonal antibody. In some embodiments, the antibody may be a human, humanized, or chimeric antibody. In some embodiments, the antibody is an antibody fragment that binds CD22. In some embodiments, the antibody binds human CD22. In some such embodiments, human CD22 has the sequence of SEQ ID NO: 28 or SEQ ID NO: 29.

In some embodiments, pharmaceutical formulations are provided, wherein the formulation comprises an immunoconjugate described herein and a pharmaceutically acceptable carrier. In some embodiments, the pharmaceutical formulation comprises an additional therapeutic agent.

In some embodiments, methods of treating an individual with a CD22-positive cancer are provided. In some embodiments, a method comprises administering to the individual an effective amount of an immunoconjugate described herein. In some embodiments, the CD22-positive cancer is selected from lymphoma, non-Hogkins lymphoma (NHL), aggressive NHL, relapsed aggressive NHL, relapsed indolent NHL, refractory NHL, refractory indolent NHL, chronic lymphocytic leukemia (CLL), small lymphocytic lymphoma, leukemia, hairy cell leukemia (HCL), acute lymphocytic leukemia (ALL), Burkitt's lymphoma, and mantle cell lymphoma. In some embodiments, the method further comprises administering an additional therapeutic agent to the individual. In some such embodiments, the additional therapeutic agent comprises an antibody that binds CD79b. In some embodiments, the additional therapeutic agent is an immunoconjugate comprising an antibody that binds CD79b covalently attached to a cytotoxic agent.

In some embodiments, a method of inhibiting proliferation of a CD22-positive cell is provided. In some such embodiments, the method comprises exposing the cell to the immunoconjugate described herein under conditions permissive for binding of the immunoconjugate to CD22 on the surface of the cell, thereby inhibiting proliferation of the cell. In some embodiments, the cell is a neoplastic B cell. In some embodiments, the cell is a lymphoma cell.

### BRIEF DESCRIPTION OF THE FIGURES

FIGS. 1A-1B: FIG. 1A shows the amino acid sequence of the heavy chain variable region of murine 10F4 anti-CD22 antibody (m10F4) aligned with the humanized 10F4 version 1 (hu10F4v1) heavy chain variable region and the humanized 10F4 version 3 (hu10F4v3) heavy chain variable region, and aligned with the human subgroup III sequence. The HVRs are boxed (HVR-H1, HVR-H2, HVR-H3). The sequences bracketing the HVRs are the framework sequences (FR-H1 to FR-H4). The sequences are numbered according to Kabat numbering. The Kabat, Chothia, and contact CDRs are indicated about the boxed HVRs. FIG. 1B shows the amino acid sequence of the light chain variable region of murine 10F4 anti-CD22 antibody (m10F4) aligned with the humanized 10F4 version 1 (hu10F4v1) light chain variable region and the humanized 10F4 version 3 (hu10F4v3) light chain variable region, and aligned with the human kappa I sequence. The antibody hu10F4v3 differ from hu10F4v1 at amino acid 28 of the HVR-L1 (N₂₈V). The HVRs are boxed. The FR-L1, FR-L2, FR-L3, and FR-L4 sequences bracket the HVRs (HVR-L1, HVR-L2, HVR-L3). The sequences are numbered according to Kabat numbering. The Kabat, Chothia, and contact CDRs are indicated about the boxed HVRs.

FIG. 2 shows the full length amino acid sequences (variable and constant regions) of the light and heavy chains of humanized anti-CD22 antibody 10F4v3, isotype IgG1. The underlined portions are the constant domains.

FIG. 3 shows the amino acid sequences of the anti-CD22 cysteine engineered antibodies in which the light chain or heavy chain or Fc region is altered to replace an amino acid with a cysteine at selected amino acid positions. The cysteine engineered antibodies shown include anti-CD22 10F4 variant light chain in which a valine at Kabat position 205 (sequential position valine 210) is altered to a cysteine (“Anti-CD22 V205C h10Fv3 Cysteine Engineered Light Chain”); anti-CD22 10F4 variant heavy chain in which an alanine at EU position 118 (sequential position alanine 121) is altered to a cysteine (“Anti-CD22 A118C h10Fv3 Cysteine Engineered Heavy Chain”); and anti-CD22 10F4 variant Fc region in which a serine at EU position 400 (sequential position serine 403) is altered to a cysteine (“Anti-CD22 S400C h10Fv3 Cysteine Engineered Fc Region”). In each figure, the altered amino acid is shown in bold text with double underlining. Single underlining indicates constant regions. Variable regions are not underlined.

FIG. 4 shows the linker and drug structure of (A) 10F4v3-PBD, (B) 10F4v3-SS-PBD, and (C) 10F4v3-SSMe-PBD, which are described in Example A.

FIG. 5 shows efficacy of various antibody-drug conjugates in a WSU-DLCL2 mouse xenograft model, as described in Example B.

FIG. 6 shows efficacy of various antibody-drug conjugates in a Granta-519 mouse xenograft model, as described in Example C.

FIG. 7 shows efficacy of various antibody-drug conjugates in a SuDHL4-luc mouse xenograft model, as described in Example D.

FIG. 8 shows dose-dependent inhibition of tumor growth by 10F4v3-PBD in a SuDHL4-luc mouse xenograft model, as described in Example E.

FIG. 9 shows dose-dependent inhibition of tumor growth by 10F4v3-PBD in a Bjab-luc mouse xenograft model, as described in Example F.

FIG. 10 shows efficacy of various antibody-drug conjugates in a WSU-DLCL2 mouse xenograft model, as described in Example G.

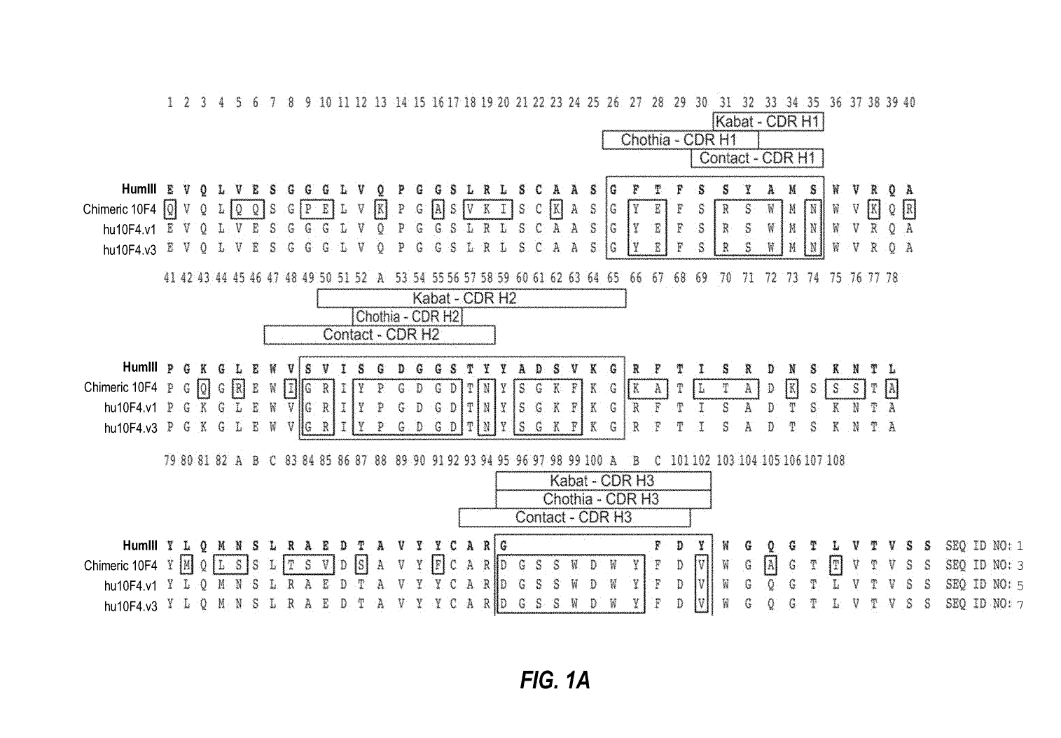

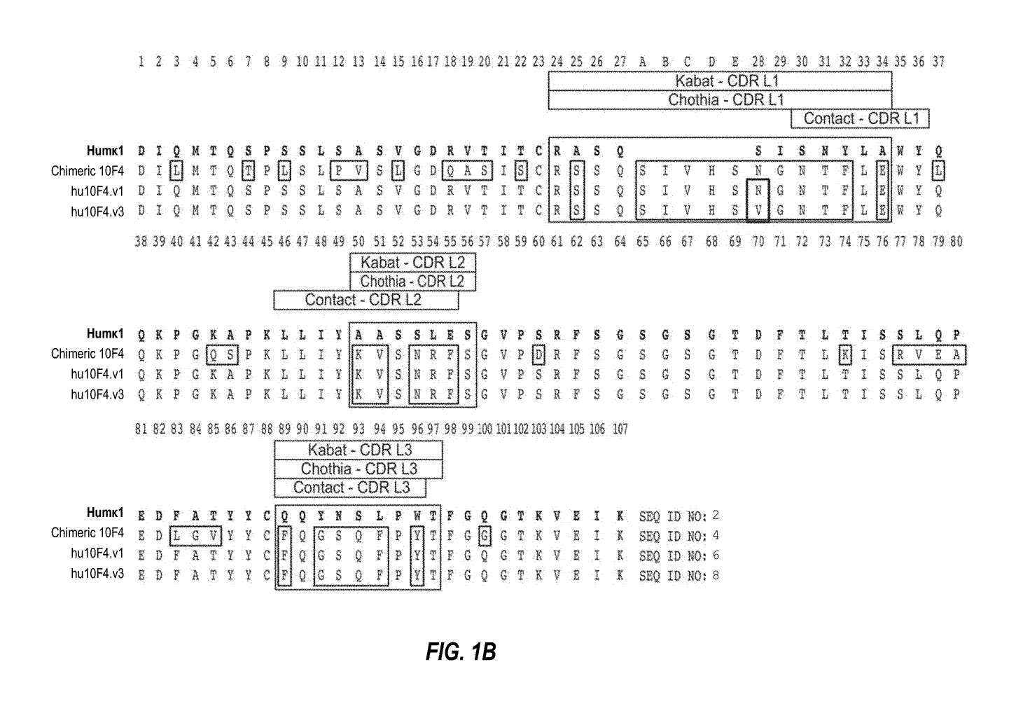

### DETAILED DESCRIPTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-CD22 antibody” and “an antibody that binds to CD22” refer to an antibody that is capable of binding CD22 with sufficient affinity such that the antibody is useful as a diagnostic and/or therapeutic agent in targeting CD22. In one embodiment, the extent of binding of an anti-CD22 antibody to an unrelated, non-CD22 protein is less than about 10% of the binding of the antibody to CD22 as measured, e.g., by a radioimmunoassay (RIA). In certain embodiments, an antibody that binds to CD22 has a dissociation constant (Kd) of ≦100 nM, ≦10 nM, ≦5 Nm, ≦4 nM, ≦3 nM, ≦2 nM, ≦1 nM, ≦0.1 nM, ≦0.01 nM, or ≦0.001 nM (e.g., 10⁻⁸ M or less, e.g. from 10⁻⁸M to 10⁻¹³M, e.g., from 10⁻⁹M to 10⁻¹³ M). In certain embodiments, an anti-CD22 antibody binds to an epitope of CD22 that is conserved among CD22 from different species.

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The terms “cancer” and “cancerous” refer to or describe the physiological condition in mammals that is typically characterized by unregulated cell growth/proliferation. Examples of cancer include, but are not limited to, melanoma, carcinoma, lymphoma (e.g., Hodgkin's and non-Hodgkin's lymphoma), blastoma, sarcoma, and leukemia. More particular examples of cancer include B-cell associated cancers, including for example, high, intermediate and low grade lymphomas (including B cell lymphomas such as, for example, mucosa-associated-lymphoid tissue B cell lymphoma and non-Hodgkin's lymphoma (NHL), mantle cell lymphoma, Burkitt's lymphoma, small lymphocytic lymphoma, marginal zone lymphoma, diffuse large cell lymphoma, follicular lymphoma, and Hodgkin's lymphoma and T cell lymphomas) and leukemias (including secondary leukemia, chronic lymphocytic leukemia (CLL), such as B cell leukemia (CD5+ B lymphocytes), myeloid leukemia, such as acute myeloid leukemia, chronic myeloid leukemia, lymphoid leukemia, such as acute lymphoblastic leukemia (ALL) and myelodysplasia), and other hematological and/or B cell- or T-cell-associated cancers. Also included are cancers of additional hematopoietic cells, including polymorphonuclear leukocytes, such as basophils, eosinophils, neutrophils and monocytes, dendritic cells, platelets, erythrocytes and natural killer cells. Also included are cancerous B cell proliferative disorders selected from the following: lymphoma, non-Hodgkins lymphoma (NHL), aggressive NHL, relapsed aggressive NHL, relapsed indolent NHL, refractory NHL, refractory indolent NHL, chronic lymphocytic leukemia (CLL), small lymphocytic lymphoma, leukemia, hairy cell leukemia (HCL), acute lymphocytic leukemia (ALL), and mantle cell lymphoma. The origins of B-cell cancers include as follows: marginal zone B-cell lymphoma origins in memory B-cells in marginal zone, follicular lymphoma and diffuse large B-cell lymphoma originates in centrocytes in the light zone of germinal centers, chronic lymphocytic leukemia and small lymphocytic leukemia originates in B1 cells (CD5+), mantle cell lymphoma originates in naive B-cells in the mantle zone and Burkitt's lymphoma originates in centroblasts in the dark zone of germinal centers. Tissues which include hematopoietic cells referred herein to as “hematopoietic cell tissues” include thymus and bone marrow and peripheral lymphoid tissues, such as spleen, lymph nodes, lymphoid tissues associated with mucosa, such as the gut-associated lymphoid tissues, tonsils, Peyer's patches and appendix and lymphoid tissues associated with other mucosa, for example, the bronchial linings. Further particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

A “B-cell malignancy” herein includes non-Hodgkin's lymphoma (NHL), including low grade/follicular NHL, small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia, non-Hodgkin's lymphoma (NHL), lymphocyte predominant Hodgkin's disease (LPHD), small lymphocytic lymphoma (SLL), chronic lymphocytic leukemia (CLL), indolent NHL including relapsed indolent NHL and rituximab-refractory indolent NHL; leukemia, including acute lymphoblastic leukemia (ALL), chronic lymphocytic leukemia (CLL), Hairy cell leukemia, chronic myeloblastic leukemia; Burkitt's lymphoma; mantle cell lymphoma; and other hematologic malignancies. Such malignancies may be treated with antibodies directed against B-cell surface markers, such as CD22. Such diseases are contemplated herein to be treated by the administration of an antibody directed against a B cell surface marker, such as CD22, and includes the administration of an unconjugated (“naked”) antibody or an antibody conjugated to a cytotoxic agent as disclosed herein. Such diseases are also contemplated herein to be treated by combination therapy including an anti-CD22 antibody or anti-CD22 antibody drug conjugate of the invention in combination with another antibody or antibody drug conjugate, another cytoxic agent, radiation or other treatment administered simultaneously or in series. In an exemplary treatment method, an anti-CD22 immunoconjugate is administered in combination with an anti-CD79b antibody, immunoglobulin, or CD79b binding fragment thereof, either together or sequentially. The anti-CD79b antibody may be a naked antibody or an antibody drug conjugate. In another exemplary treatment method, an anti-CD22 immunoconjugate is administered in combination with an anti-CD20 antibody, immunoglobulin, or CD20 binding fragment thereof, either together or sequentially. The anti-CD20 antibody may be a naked antibody or an antibody drug conjugate. In some embodiments of the combination therapy, the anti-CD22 immunoconjugate is administered with Rituxan® (rituximab).

The term “non-Hodgkin's lymphoma” or “NHL”, as used herein, refers to a cancer of the lymphatic system other than Hodgkin's lymphomas. Hodgkin's lymphomas can generally be distinguished from non-Hodgkin's lymphomas by the presence of Reed-Sternberg cells in Hodgkin's lymphomas and the absence of said cells in non-Hodgkin's lymphomas. Examples of non-Hodgkin's lymphomas encompassed by the term as used herein include any that would be identified as such by one skilled in the art (e.g., an oncologist or pathologist) in accordance with classification schemes known in the art, such as the Revised European-American Lymphoma (REAL) scheme as described in Color Atlas of Clinical Hematology (3rd edition), A. Victor Hoffbrand and John E. Pettit (eds.) (Harcourt Publishers Ltd., 2000). See, in particular, the lists in FIGS. 11.57, 11.58 and 11.59. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B cell lymphoma, splenic marginal zone lymphoma, extranodal marginal zone—MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®; Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and docetaxel (TAXOTERE®; Rhone-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone; CVP, an abbreviation for a combined therapy of cyclophosphamide, vincristine, and prednisolone; and FOLFOX, an abbreviation for a treatment regimen with oxaliplatin (ELOXATIN™) combined with 5-FU and leucovorin.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “epitope” refers to the particular site on an antigen molecule to which an antibody binds.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The term “glycosylated forms of CD22” refers to naturally occurring forms of CD22 that are post-translationally modified by the addition of carbohydrate residues.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated antibody” is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-CD22 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “CD22,” as used herein, refers to any native CD22 from any vertebrate source, including mammals such as primates (e.g. humans, cynomolgus monkey (cyno)) and rodents (e.g., mice and rats), unless otherwise indicated. The term encompasses “full-length,” unprocessed CD22 as well as any form of CD22 that results from processing in the cell. The term also encompasses naturally occurring variants of CD22, e.g., splice variants, allelic variants, and isoforms. The major isoform of CD22 (CD22beta) comprises 847 amino acids and seven immunoglobulin-like regions in the extracellular domain (see Wilson, G. L. et al., J. Exp. Med. 173:137-146 (1991)). A minor isoform, CD22alpha, comprises 647 amino acids and lacks immunoglobulin-like domains 3 and 4 in the extracellular domain (see Stamenkovic, I. and Seed, B., Nature 345:74-77 (1990)) and Wilson et al. (1991), supra). The amino acid sequence of an exemplary human CD22beta precursor (with signal sequence) is shown in SEQ ID NO: 28. The amino acid sequence of an exemplary human mature CD22beta (without signal sequence) is shown in SEQ ID NO: 29. The amino acid sequence of an exemplary human CD22alpha precursor (with signal sequence) is shown in SEQ ID NO: 30. The amino acid sequence of an exemplary human mature CD22alpha (without signal sequence) is shown in SEQ ID NO: 31.

The term “CD22-positive cancer” refers to a cancer comprising cells that express CD22 on their surface.

The term “CD22-positive cell” refers to a cell that expresses CD22 on its surface.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH₁, CH₂, and CH₃). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, immunoconjugates of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The phrase “optionally substituted” as used herein, pertains to a parent group that may be unsubstituted or that may be substituted.

Unless otherwise specified, the term “substituted” as used herein, pertains to a parent group that bears one or more substituents. The term “substituent” is used herein in the conventional sense and refers to a chemical moiety that is covalently attached to, or if appropriate, fused to, a parent group. A wide variety of substituents are known, and methods for their formation and introduction into a variety of parent groups are also known.

In some embodiments, the substituents described herein (which include optional substituents) are limited to those groups that are not reactive to the antibody. In some embodiments, the link to the antibody is formed from the N10 position of the PBD compound through the linker (L). In some instances, reactive functional groups located at other parts of the PBD structure may be capable of forming additional bonds to the antibody (this may be referred to as crosslinking). Such additional bonds, in some instances, may alter transport and biological activity of the conjugate. Therefore, in some embodiments, the additional substituents are limited to those lacking reactive functionality.

In some embodiments, the substituents are selected from R, OR, SR, NRR′, NO₂, halo, CO₂R, COR, CONH₂, CONHR, and CONRR′. In some embodiments, the substituents are selected from R, OR, SR, NRR′, NO₂, CO₂R, COR, CONH₂, CONHR, and CONRR′. In some embodiments, the substituents are selected from R, OR, SR, NRR′, NO₂, and halo. In some embodiments, the substituents are selected from the group consisting of R, OR, SR, NRR′, and NO₂.

Any of the embodiments discussed above may be applied to any of the substituents described herein. Alternatively, the substituents may be selected from one or more of the groups discussed below.

The term “C₁₋₁₂ alkyl” as used herein, pertains to a monovalent moiety obtained by removing a hydrogen atom from a carbon atom of a hydrocarbon compound having from 1 to 12 carbon atoms, which is aliphatic, and which may be cyclic or acyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇).

Examples of saturated branched alkyl groups include, but are not limited to, iso-propyl (C₃), iso-butyl (C₄), sec-butyl (C₄), tert-butyl (C₄), iso-pentyl (C₅), and neo-pentyl (C₅).

An alkyl group may optionally be interrupted by one or more heteroatoms selected from O, N(H) and S. Such groups may be referred to as “heteroalkyl”.

The term “C₂₋₁₂ heteroalkyl” as used herein, pertains to a monovalent moiety obtained by removing a hydrogen atom from a carbon atom of a hydrocarbon compound having from 2 to 12 carbon atoms, and one or more heteroatoms selected from O, N(H) and S, preferably O and S.

Examples of heteroalkyl groups include, but are not limited to, those comprising one or more ethylene glycol units of the type —(OCH₂CH₂)—. The terminus of a heteroalkyl group may be the primary form of a heteroatom, e.g. —OH, —SH or —NH₂. In a preferred embodiment, the terminus is —CH₃.

The term “C₂₋₁₂ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

The term “C₂₋₁₂ alkynyl” as used herein, pertains to an alkyl group having one or more carbon-carbon triple bonds.

Examples of unsaturated alkynyl groups include, but are not limited to, ethynyl (—C≡CH) and 2-propynyl (propargyl, —CH₂C≡CH).

The term “C₃₋₁₂ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:

(i) saturated monocyclic hydrocarbon compounds:
cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);

(ii) unsaturated monocyclic hydrocarbon compounds:
cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and

(iii) saturated polycyclic hydrocarbon compounds:
norcarane (C₇), norpinane (C₇), norbornane (C₇).

The term “C₃₋₂₀ heterocyclyl” as used herein, pertains to a monovalent moiety obtained by removing a hydrogen atom from a ring atom of a heterocyclic compound, which moiety has from 3 to 20 ring atoms, of which from 1 to 10 are ring heteroatoms. In some embodiments, each ring has from 3 to 7 ring atoms, of which from 1 to 4 are ring heteroatoms.

As used herein, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms.

Examples of monocyclic heterocyclyl groups include, but are not limited to, those derived from:
(i) N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
(ii) O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
(iii) S1: thiirane (C₃), thietane (C₄), thiolane (tetrahydrothiophene) (C₅), thiane (tetrahydrothiopyran) (C₆), thiepane (C₇);
(iv) O₂: dioxolane (C₅), dioxane (C₆), and dioxepane (C₇);
(v) O₃: trioxane (C₆);
(vi) N₂: imidazolidine (C₅), pyrazolidine (diazolidine) (C₅), imidazoline (C₅), pyrazoline (dihydropyrazole) (C₅), piperazine (C₆);
(vii) N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
(viii) N₁S₁: thiazoline (C₅), thiazolidine (C₅), thiomorpholine (C₆);
(ix) N₂O₁: oxadiazine (C₆);
(x) O₁S₁: oxathiole (C₅) and oxathiane (thioxane) (C₆); and,
(xi) N₁O₁S₁: oxathiazine (C₆).

Examples of substituted monocyclic heterocyclyl groups include, but are not limited to, those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

The term “C₅₋₂₀ aryl”, as used herein, pertains to a monovalent moiety obtained by removing a hydrogen atom from an aromatic ring atom of an aromatic compound, which moiety has from 3 to 20 ring atoms. In some embodiments, each ring has from 5 to 7 ring atoms.

In some embodiments, the ring atoms are all carbon atoms, as in “carboaryl groups”. Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₀), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

In some embodiments, the ring atoms may include one or more heteroatoms, as in “heteroaryl groups”. Examples of monocyclic heteroaryl groups include, but are not limited to, those derived from:
(i) N₁: pyrrole (azole) (C₅), pyridine (azine) (C₆);
(ii) O₁: furan (oxole) (C₅);
(iii) S₁: thiophene (thiole) (C₅);
(iv) N₁O₁: oxazole (C₅), isoxazole (C₅), isoxazine (C₆);
(v) N₂O₁: oxadiazole (furazan) (C₅);
(vi) N₃O₁: oxatriazole (C₅);
(vii) N₁S₁: thiazole (C₅), isothiazole (C₅);
(viii) N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

(ix) N₃: triazole (C₅), triazine (C₆); and,

(x) N₄: tetrazole (C₅).

Examples of heteroaryl which comprise fused rings, include, but are not limited to:
(i) C₉ (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
(ii) C₁₀ (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
(iii) C₁₁ (with 2 fused rings) derived from benzodiazepine (N₂);
(iv) C₁₃ (with 3 fused rings) derived from carbazole (N₁), dibenzofuran (O₁), dibenzothiophene (S₁), carboline (N₂), perimidine (N₂), pyridoindole (N₂); and,
(v) C₁₄ (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

The above groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀heterocyclyloxy group), or a C₅₋₂₀ aryl group (also referred to as a C₅₋₂₀ aryloxy group). In some embodiments, R is a C₁₋₇ alkyl group.

Alkoxy: —OR, wherein R is an alkyl group, for example, a C₁₋₇ alkyl group. Examples of C₁₋₇ alkoxy groups include, but are not limited to, -OMe (methoxy), -OEt (ethoxy), -O(nPr) (n-propoxy), -O(iPr) (isopropoxy), -O(nBu) (n-butoxy), -O(sBu) (sec-butoxy), -O(iBu) (isobutoxy), and -O(tBu) (tert-butoxy).

Acetal: —CH(OR¹)(OR²), wherein R¹ and R² are independently acetal substituents, for example, a C₁₋₇ alkyl group, a C₃₋₂₀ heterocyclyl group, or a C₅₋₂₀ aryl group. In some embodiments, R¹ and/or R² are independently a C₁₋₇ alkyl group. In some embodiments, in the case of a “cyclic” acetal group, R¹ and R², taken together with the two oxygen atoms to which they are attached, and the carbon atom to which they are attached, form a heterocyclic ring having from 4 to 8 ring atoms. Examples of acetal groups include, but are not limited to, —CH(OMe)₂, —CH(OEt)₂, and —CH(OMe)(OEt).

Hemiacetal: —CH(OH)(OR¹), wherein R¹ is a hemiacetal substituent, for example, a C₁₋₇ alkyl group, a C₃₋₂₀ heterocyclyl group, or a C₅₋₂₀ aryl group. In some embodiments, R¹ is a C₁₋₇ alkyl group. Examples of hemiacetal groups include, but are not limited to, —CH(OH)(OMe) and —CH(OH)(OEt).

Ketal: —CR(OR¹)(OR²), where R¹ and R² are as defined for acetals, and R is a ketal substituent other than hydrogen, for example, a C₁₋₇ alkyl group, a C₃₋₂₀ heterocyclyl group, or a C₅₋₂₀ aryl group. In some embodiments, R is a C₁₋₇ alkyl group. Examples ketal groups include, but are not limited to, —C(Me)(OMe)₂, —C(Me)(OEt)₂, —C(Me)(OMe)(OEt), —C(Et)(OMe)₂, —C(Et)(OEt)₂, and —C(Et)(OMe)(OEt).

Hemiketal: —CR(OH)(OR¹), where R¹ is as defined for hemiacetals, and R is a hemiketal substituent other than hydrogen, for example, a C₁₋₇ alkyl group, a C₃₋₂₀ heterocyclyl group, or a C₅₋₂₀ aryl group. In some embodiments, R is a C₁₋₇ alkyl group. Examples of hemiketal groups include, but are not limited to, —C(Me)(OH)OMe), —C(Et)(OH)OMe), —C(Me)(OH)(OEt), and —C(Et)(OH)(OEt).

Oxo (keto, -one): ═O.

Thione (thioketone): ═S.

Imino (imine): ═NR, wherein R is an imino substituent, for example, hydrogen, C₁₋₇ alkyl group, a C₃₋₂₀ heterocyclyl group, or a C₅₋₂₀ aryl group. In some embodiments, R is hydrogen or a C₁₋₇ alkyl group. Examples of imino groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl). In some embodiments, R is a C₁₋₇ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group. In some embodiments, R is a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group. In some embodiments, R is a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarbonyloxy: —OC(═O)OR, wherein R is an ester substituent, for example, a C₁₋₇ alkyl group, a C₃₋₂₀ heterocyclyl group, or a C₅₋₂₀ aryl group. In some embodiments, R is a C₁₋₇ alkyl group. Examples of oxycarbonyloxy groups include, but are not limited to, —OC(═O)OCH₃, —OC(═O)OCH₂CH₃, —OC(═O)OC(CH₃)₃, and —OC(═O)OPh.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group. In some embodiments, R¹ and R² are independently H or a C₁₋₇ alkyl group. In some embodiments, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR¹R², wherein R¹ and R² are independently amino substituents, as defined for amino groups. Examples of thioamido groups include, but are not limited to, —C(═S)NH₂, —C(═S)NHCH₃, —C(═S)N(CH₃)₂, and —C(═S)NHCH₂CH₃.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, and R² is an acyl substituent, for example, a C₁₋₇ alkyl group, a C₃₋₂₀ heterocyclyl group, or a C₅₋₂₀aryl group. In some embodiments, R¹ and/or R² is hydrogen or a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

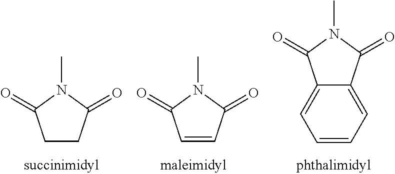

Aminocarbonyloxy: —OC(═O)NR¹R², wherein R¹ and R² are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R¹)CONR²R³ wherein R² and R³ are independently amino substituents, as defined for amino groups, and R¹ is a ureido substituent, for example, hydrogen, a C₁₋₇ alkyl group, a C₃₋₂₀ heterocyclyl group, or a C₅₋₂₀ aryl group. In some embodiments, R¹ is hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

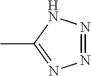

Amidine (amidino): —C(═NR)NR₂, wherein each R is an amidine substituent, for example, hydrogen, a C₁₋₇ alkyl group, a C₃₋₂₀ heterocyclyl group, or a C₅₋₂₀ aryl group. In some embodiments, each R is H or a C₁₋₇ alkyl group. Examples of amidine groups include, but are not limited to, —C(═NH)NH₂, —C(═NH)NMe₂, and —C(═NMe)NMe₂.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇alkylthio group), a C₃₋₂₀ heterocyclyl group, or a C₅₋₂₀ aryl group. In some embodiments, R is a C₁₋₇ alkyl group. Examples of thioether groups include, but are not limited to, —SCH₃ and —SCH₂CH₃.

Disulfide: —SS—R, wherein R is a disulfide substituent, for example, a C₁₋₇ alkyl group, a C₃₋₂₀ heterocyclyl group, or a C₅₋₂₀ aryl group. In some embodiments, R is a C₁₋₇ alkyl group (also referred to herein as C₁₋₇ alkyl disulfide). Examples of disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₉ aryl group. In some embodiments, R is a C₁₋₇ alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group. In some embodiments, R is a C₁₋₇ alkyl group, including, for example, a fluorinated or perfluorinated C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇ alkyl group, a C₃₋₂₀ heterocyclyl group, or a C₅₋₂₀ aryl group. In some embodiments, R is a C₁₋₇ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇ alkyl group, a C₃₋₂₀ heterocyclyl group, or a C₅₋₂₀ aryl group. In some embodiments, R is a C₁₋₇ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group. In some embodiments, R is a C₁₋₇ alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group. In some embodiments, R is a C₁₋₇ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇ alkyl group, a C₃₋₂₀ heterocyclyl group, or a C₅₋₂₀ aryl group. In some embodiments, R is a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH 3)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group. In some embodiments, R is a C₁₋₇ alkyl group. Examples of sulfonamino groups include, but are not limited to, —NHS(═O)₂CH₃ and —N(CH₃)S(═O)₂C₆H₅.

Sulfinamino: —NR¹S(═O)₂R, wherein R¹ is an amino substituent, as defined for amino groups, and R is a sulfinamino substituent, for example, a C₁₋₇ alkyl group, a C₃₋₂₀ heterocyclyl group, or a C₅₋₂₀ aryl group. In some embodiments, R is a C₁₋₇ alkyl group. Examples of sulfinamino groups include, but are not limited to, —NHS(═O)CH₃ and —N(CH₃)S(═O)C₆H₅.

Phosphino (phosphine): —PR₂, wherein R is a phosphino substituent, for example, —H, a C₁₋₇ alkyl group, a C₃₋₂₀ heterocyclyl group, or a C₅₋₂₀ aryl group. In some embodiments, R is —H, a C₁₋₇ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂.

Phospho: —P(═O)₂.

Phosphinyl (phosphine oxide): —P(═O)R₂, wherein R is a phosphinyl substituent, for example, a C₁₋₇ alkyl group, a C₃₋₂₀ heterocyclyl group, or a C₅₋₂₀ aryl group. In some embodiments, R is a C₁₋₇ alkyl group or a C₅₋₂₀ aryl group. Examples of phosphinyl groups include, but are not limited to, —P(═O)(CH₃)₂, —P(═O)(CH₂CH₃)₂, —P(═O)(t-Bu)₂, and —P(═O)(Ph)₂.

Phosphonic acid (phosphono): —P(═O)(OH)₂.

Phosphonate (phosphono ester): —P(═O)(OR)₂, where R is a phosphonate substituent, for example, —H, a C₁₋₇ alkyl group, a C₃₋₂₀ heterocyclyl group, or a C₅₋₂₀ aryl group. In some embodiments, R is —H, a C₁₋₇ alkyl group, or a C₅₋₂₀ aryl group. Examples of phosphonate groups include, but are not limited to, —P(═O)(OCH₃)₂, —P(═O)(OCH₂CH₃)₂, —P(═O)(0-t-Bu)₂, and —P(═O)(OPh)₂.

Phosphoric acid (phosphonooxy): —OP(═O)(OH)₂.

Phosphate (phosphonooxy ester): —OP(═O)(OR)₂, where R is a phosphate substituent, for example, —H, a C₁₋₇ alkyl group, a C₃₋₂₀ heterocyclyl group, or a C₅₋₂₀ aryl group. In some embodiments, R is —H, a C₁₋₇ alkyl group, or a C₅₋₂₀ aryl group. Examples of phosphate groups include, but are not limited to, —OP(═O)(OCH₃)₂, —OP(═O)(OCH₂CH₃)₂, —OP(═O)(0-t-Bu)₂, and —OP(═O)(OPh)₂.

Phosphorous acid: —OP(OH)₂

Phosphite: —OP(OR)₂, where R is a phosphite substituent, for example, —H, a C₁₋₇ alkyl group, a C₃₋₂₀ heterocyclyl group, or a C₅₋₂₀ aryl group. In some embodiments, R is —H, a C₁₋₇ alkyl group, or a C₅₋₂₀ aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR¹)—NR²₂, where R¹ and R² are phosphoramidite substituents, for example, —H, a (optionally substituted) C₁₋₇ alkyl group, a C₃₋₂₀ heterocyclyl group, or a C₅₋₂₀ aryl group. In some embodiments, R is —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR¹)—NR²₂, where R¹ and R² are phosphoramidate substituents, for example, —H, a (optionally substituted) C₁₋₇ alkyl group, a C₃₋₂₀ heterocyclyl group, or a C₅₋₂₀ aryl group. In some embodiments, R¹ and R² are —H, a C₁₋₇ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

The term “C₃₋₁₂ alkylene”, as used herein, pertains to a bidentate moiety obtained by removing two hydrogen atoms, either both from the same carbon atom, or one from each of two different carbon atoms, of a hydrocarbon compound having from 3 to 12 carbon atoms (unless otherwise specified), which is aliphatic, and which may be cyclic or acyclic, and which may be saturated, partially unsaturated, or fully unsaturated. Thus, the term “alkylene” includes the sub-classes alkenylene, alkynylene, cycloalkylene, etc., discussed below.

Examples of linear saturated C₃₋₁₂ alkylene groups include, but are not limited to, —(CH₂)ₙ— where n is an integer from 3 to 12, for example, —CH₂CH₂CH₂— (propylene), —CH₂CH₂CH₂CH₂— (butylene), —CH₂CH₂CH₂CH₂CH₂— (pentylene) and —CH₂CH₂CH₂CH₂CH₂CH₂CH₂— (heptylene).

Examples of branched saturated C₃₋₁₂ alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂C H(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated C₃₋₁₂ alkylene groups (C₃₋₁₂ cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

“Linker” refers to a chemical moiety comprising a covalent bond or a chain of atoms that covalently attaches an antibody to a drug moiety. Nonlimiting exemplary linkers are described herein.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Leaving group” refers to a functional group that can be substituted by another functional group. Certain leaving groups are well known in the art, and examples include, but are not limited to, a halide (e.g., chloride, bromide, iodide), methanesulfonyl (mesyl), p-toluenesulfonyl (tosyl), trifluoromethylsulfonyl (triflate), and trifluoromethylsulfonate.

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBZ) and 9-fluorenylmethylenoxycarbonyl (Fmoc). For a general description of protecting groups and their use, see T. W. Greene, Protective Groups in Organic Synthesis, John Wiley & Sons, New York, 1991, or a later edition.

### II. Compositions and Methods

In one aspect, the invention is based, in part, on antibodies that bind to CD22 and immunoconjugates comprising such antibodies. Antibodies and immunoconjugates of the invention are useful, e.g., for the diagnosis or treatment of CD22-positive cancers.

### A. Exemplary Anti-CD22 Antibodies

In some embodiments, isolated antibodies that bind to CD22 are provided. CD22 is a 135-kDa B-cell-restricted sialoglycoprotein expressed on the B-cell surface at the mature stages of differentiation. CD22 is expressed in various B-cell related disorders and cancers, including various lymphomas, such as Non-Hodgkin's lymphoma.

An exemplary naturally occurring human CD22 precursor sequence, with signal sequence (amino acids 1 to 19) is provided in SEQ ID NO: 28, and the corresponding mature CD22 sequence is shown in SEQ ID NO: 29 (corresponding to amino acids 20 to 847 of SEQ ID NO: 28). A further exemplary naturally occurring human CD22 precursor sequence, with signal sequence (amino acids 1 to 19) is provided in SEQ ID NO: 30, and the corresponding mature CD22 sequence is shown in SEQ ID NO: 31 (corresponding to amino acids 20 to 670 of SEQ ID NO: 30).

In certain embodiments, an anti-CD22 antibody binds an epitope within amino acids 20 to 240 of SEQ ID NO: 28. Nonlimiting exemplary such antibodies include 10F4 and humanized versions thereof. In some embodiments, an anti-CD22 antibody binds human CD22. In some embodiments, an anti-CD22 antibody binds human CD22 and cynomolgus monkey CD22.

In some embodiments, an anti-CD22 antibody binds human CD22 with an affinity of ≦10 nM, or ≦5 nM, or ≦4 nM, or ≦3 nM, or ≦2 nM and optionally ≧0.0001 nM, or ≧0.001 nM, or ≧0.01 nM. Nonlimiting exemplary such antibodies include mu10F4, hu10F4v1, and hu10F4v3, which bind to human CD22 with an affinity of 2.4 nM, 1.1-1.7 nM, and 1.6 nM, respectively. See, e.g., US 2008/0050310.

Assays

To determine whether an anti-CD22 antibody “binds to an epitope within amino acids 20 to 240 of SEQ ID NO: 28,” CD22 polypeptides with N- and C-terminal deletions are expressed in CHO cells and binding of the antibody to the truncated polypeptides is tested by FACS as described previously. See, e.g., US 2008/0050310. A substantial reduction (≧70% reduction) or elimination of binding of the antibody to a truncated polypeptide relative to binding to full-length CD22 expressed in CHO cells indicates that the antibody does not bind to that truncated polypeptide.

Whether an anti-CD22 antibody “binds with an affinity of” ≦10 nM, or ≦5 nM, or ≦4 nM, or ≦3 nM, or ≦2 nM, is determined using CHO cells expressing CD22 on the surface in a competition assay using serially diluted, unlabeled anti-CD22 antibody. See, e.g., US 2008/0050310. Binding affinity, K_{D}, of the antibodies may be determined in accordance with standard Scatchard analysis performed utilizing a non-linear curve fitting program (see, for example, Munson et al., Anal Biochem, 107: 220-239, 1980).

### Antibody 10F4 and Other Embodiments

In some embodiments, the invention provides an anti-CD22 antibody or immunoconjugate comprising at least one, two, three, four, five, or six HVRs selected from (a) HVR-H1 comprising the amino acid sequence of SEQ ID NO: 9; (b) HVR-H2 comprising the amino acid sequence of SEQ ID NO: 10; (c) HVR-H3 comprising the amino acid sequence of SEQ ID NO: 11; (d) HVR-L1 comprising an amino acid sequence selected from SEQ ID NOs: 12 and 15 to 22; (e) HVR-L2 comprising the amino acid sequence of SEQ ID NO: 13; and (0 HVR-L3 comprising the amino acid sequence of SEQ ID NO: 14. In some embodiments, the invention provides an anti-CD22 antibody or immunoconjugate comprising at least one, two, three, four, five, or six HVRs selected from (a) HVR-H1 comprising the amino acid sequence of SEQ ID NO: 9; (b) HVR-H2 comprising the amino acid sequence of SEQ ID NO: 10; (c) HVR-H3 comprising the amino acid sequence of SEQ ID NO: 11; (d) HVR-L1 comprising an amino acid sequence of SEQ ID NO: 15; (e) HVR-L2 comprising the amino acid sequence of SEQ ID NO: 13; and (0 HVR-L3 comprising the amino acid sequence of SEQ ID NO: 14.

In one aspect, the invention provides an antibody or immunoconjugate comprising at least one, at least two, or all three VH HVR sequences selected from (a) HVR-H1 comprising the amino acid sequence of SEQ ID NO: 9; (b) HVR-H2 comprising the amino acid sequence of SEQ ID NO: 10; and (c) HVR-H3 comprising the amino acid sequence of SEQ ID NO: 11. In one embodiment, the antibody comprises HVR-H3 comprising the amino acid sequence of SEQ ID NO: 11. In another embodiment, the antibody comprises HVR-H3 comprising the amino acid sequence of SEQ ID NO: 11 and HVR-L3 comprising the amino acid sequence of SEQ ID NO: 14. In a further embodiment, the antibody comprises HVR-H3 comprising the amino acid sequence of SEQ ID NO: 11, HVR-L3 comprising the amino acid sequence of SEQ ID NO: 14, and HVR-H2 comprising the amino acid sequence of SEQ ID NO: 10. In a further embodiment, the antibody comprises (a) HVR-H1 comprising the amino acid sequence of SEQ ID NO: 9; (b) HVR-H2 comprising the amino acid sequence of SEQ ID NO: 10; and (c) HVR-H3 comprising the amino acid sequence of SEQ ID NO: 11.

In another aspect, the invention provides an antibody or immunoconjugate comprising at least one, at least two, or all three VL HVR sequences selected from (a) HVR-L1 comprising an amino acid sequence selected from SEQ ID NOs: 12 and 15 to 22; (b) HVR-L2 comprising the amino acid sequence of SEQ ID NO: 13; and (c) HVR-L3 comprising the amino acid sequence of SEQ ID NO: 14. In another aspect, the invention provides an antibody or immunoconjugate comprising at least one, at least two, or all three VL HVR sequences selected from (a) HVR-L1 comprising the amino acid sequence of SEQ ID NO: 15; (b) HVR-L2 comprising the amino acid sequence of SEQ ID NO: 13; and (c) HVR-L3 comprising the amino acid sequence of SEQ ID NO: 14. In one embodiment, the antibody comprises (a) HVR-L1 comprising an amino acid sequence selected from SEQ ID NOs: 12 and 15 to 22; (b) HVR-L2 comprising the amino acid sequence of SEQ ID NO: 13; and (c) HVR-L3 comprising the amino acid sequence of SEQ ID NO: 14. In one embodiment, the antibody comprises (a) HVR-L1 comprising the amino acid sequence of SEQ ID NO: 15; (b) HVR-L2 comprising the amino acid sequence of SEQ ID NO: 13; and (c) HVR-L3 comprising the amino acid sequence of SEQ ID NO: 14.

In another aspect, an antibody of the invention or immunoconjugate comprises (a) a VH domain comprising at least one, at least two, or all three VH HVR sequences selected from (i) HVR-H1 comprising the amino acid sequence of SEQ ID NO: 9, (ii) HVR-H2 comprising the amino acid sequence of SEQ ID NO: 10, and (iii) HVR-H3 comprising an amino acid sequence selected from SEQ ID NO: 11; and (b) a VL domain comprising at least one, at least two, or all three VL HVR sequences selected from (i) HVR-L1 comprising an amino acid sequence selected from SEQ ID NOs: 12 and 15 to 22, (ii) HVR-L2 comprising the amino acid sequence of SEQ ID NO: 13, and (c) HVR-L3 comprising the amino acid sequence of SEQ ID NO: 14. In another aspect, an antibody or immunoconjugate of the invention comprises (a) a VH domain comprising at least one, at least two, or all three VH HVR sequences selected from (i) HVR-H1 comprising the amino acid sequence of SEQ ID NO: 9, (ii) HVR-H2 comprising the amino acid sequence of SEQ ID NO: 10, and (iii) HVR-H3 comprising an amino acid sequence selected from SEQ ID NO: 11; and (b) a VL domain comprising at least one, at least two, or all three VL HVR sequences selected from (i) HVR-L1 comprising the amino acid sequence of SEQ ID NO: 15, (ii) HVR-L2 comprising the amino acid sequence of SEQ ID NO: 13, and (c) HVR-L3 comprising the amino acid sequence of SEQ ID NO: 14.

In another aspect, the invention provides an antibody or immunoconjugate comprising (a) HVR-H1 comprising the amino acid sequence of SEQ ID NO: 9; (b) HVR-H2 comprising the amino acid sequence of SEQ ID NO: 10; (c) HVR-H3 comprising the amino acid sequence of SEQ ID NO: 11; (d) HVR-L1 comprising an amino acid sequence selected from SEQ ID NOs: 12 and 15 to 22; (e) HVR-L2 comprising the amino acid sequence of SEQ ID NO: 13; and (f) HVR-L3 comprising the amino acid sequence of SEQ ID NO: 14. In another aspect, the invention provides an antibody or immunoconjugate comprising (a) HVR-H1 comprising the amino acid sequence of SEQ ID NO: 9; (b) HVR-H2 comprising the amino acid sequence of SEQ ID NO: 10; (c) HVR-H3 comprising the amino acid sequence of SEQ ID NO: 11; (d) HVR-L1 comprising the amino acid sequence of SEQ ID NO: 15; (e) HVR-L2 comprising the amino acid sequence of SEQ ID NO: 13; and (f) HVR-L3 comprising the amino acid sequence of SEQ ID NO: 14.

In any of the above embodiments, an anti-CD22 antibody is humanized. In one embodiment, an anti-CD22 antibody comprises HVRs as in any of the above embodiments, and further comprises a human acceptor framework, e.g. a human immunoglobulin framework or a human consensus framework. In certain embodiments, the human acceptor framework is the human VL kappa 1 (VL_{KI}) framework and/or the VH framework VH_{III}. In some embodiments, a humanized anti-CD22 antibody comprises (a) HVR-H1 comprising the amino acid sequence of SEQ ID NO: 9; (b) HVR-H2 comprising the amino acid sequence of SEQ ID NO: 10; (c) HVR-H3 comprising the amino acid sequence of SEQ ID NO: 11; (d) HVR-L1 comprising an amino acid sequence selected from SEQ ID NOs: 12 and 15 to 22; (e) HVR-L2 comprising the amino acid sequence of SEQ ID NO: 13; and (0 HVR-L3 comprising the amino acid sequence of SEQ ID NO: 14. In some embodiments, a humanized anti-CD22 antibody comprises (a) HVR-H1 comprising the amino acid sequence of SEQ ID NO: 9; (b) HVR-H2 comprising the amino acid sequence of SEQ ID NO: 10; (c) HVR-H3 comprising the amino acid sequence of SEQ ID NO: 11; (d) HVR-L1 comprising the amino acid sequence of SEQ ID NO: 15; (e) HVR-L2 comprising the amino acid sequence of SEQ ID NO: 13; and (0 HVR-L3 comprising the amino acid sequence of SEQ ID NO: 14.

In another aspect, an anti-CD22 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 7. In certain embodiments, a VH sequence having at least 90%, 91%, 92%, 93%, 94%, 95%, 96%, 97%, 98%, or 99% identity to the amino acid sequence of SEQ ID NO: 7 contains substitutions (e.g., conservative substitutions), insertions, or deletions relative to the reference sequence, but an anti-CD22 antibody comprising that sequence retains the ability to bind to CD22. In certain embodiments, a total of 1 to 10 amino acids have been substituted, inserted and/or deleted in SEQ ID NO: 7. In certain embodiments, a total of 1 to 5 amino acids have been substituted, inserted and/or deleted in SEQ ID NO: 7. In certain embodiments, substitutions, insertions, or deletions occur in regions outside the HVRs (i.e., in the FRs).

Optionally, the anti-CD22 antibody comprises the VH sequence of SEQ ID NO: 5 or SEQ ID NO: 7, including post-translational modifications of that sequence. In a particular embodiment, the VH comprises one, two or three HVRs selected from: (a) HVR-H1 comprising the amino acid sequence of SEQ ID NO: 9, (b) HVR-H2 comprising the amino acid sequence of SEQ ID NO: 10, and (c) HVR-H3 comprising the amino acid sequence of SEQ ID NO: 11.

In some embodiments, an anti-CD22 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 8. In certain embodiments, a VL sequence having at least 90%, 91%, 92%, 93%, 94%, 95%, 96%, 97%, 98%, or 99% identity to the amino acid sequence of SEQ ID NO: 8 contains substitutions (e.g., conservative substitutions), insertions, or deletions relative to the reference sequence, but an anti-CD22 antibody comprising that sequence retains the ability to bind to CD22. In certain embodiments, a total of 1 to 10 amino acids have been substituted, inserted and/or deleted in SEQ ID NO: 8. In certain embodiments, a total of 1 to 5 amino acids have been substituted, inserted and/or deleted in SEQ ID NO: 8. In certain embodiments, the substitutions, insertions, or deletions occur in regions outside the HVRs (i.e., in the FRs). Optionally, the anti-CD22 antibody comprises the VL sequence of SEQ ID NO: 6 or SEQ ID NO: 8, including post-translational modifications of that sequence. In a particular embodiment, the VL comprises one, two or three HVRs selected from (a) HVR-L1 comprising the amino acid sequence of SEQ ID NO: 15; (b) HVR-L2 comprising the amino acid sequence of SEQ ID NO: 13; and (c) HVR-L3 comprising the amino acid sequence of SEQ ID NO: 14. In some embodiments, the VL comprises one, two or three HVRs selected from (a) HVR-L1 comprising an amino acid sequence selected from SEQ ID NOs: 12 and 15 to 22; (b) HVR-L2 comprising the amino acid sequence of SEQ ID NO: 13; and (c) HVR-L3 comprising the amino acid sequence of SEQ ID NO: 14.

In another aspect, an anti-CD22 antibody is provided, wherein the antibody comprises a VH as in any of the embodiments provided above, and a VL as in any of the embodiments provided above. In some embodiments, the antibody comprises the VH and VL sequences in SEQ ID NO: 7 and SEQ ID NO: 8, respectively, including post-translational modifications of those sequences. In some embodiments, the antibody comprises the VH and VL sequences in SEQ ID NO: 5 and SEQ ID NO: 6, respectively, including post-translational modifications of those sequences. In some embodiments, the antibody comprises the heavy chain and light chain sequences in SEQ ID NO: 24 and SEQ ID NO: 23, respectively, including post-translational modifications of those sequences. In some embodiments, the antibody comprises the heavy chain and light chain sequences in SEQ ID NO: 26 and SEQ ID NO: 23, respectively, including post-translational modifications of those sequences. In some embodiments, the antibody comprises the heavy chain and light chain sequences in SEQ ID NO: 25 and SEQ ID NO: 23, respectively, including post-translational modifications of those sequences. In some embodiments, the antibody comprises the heavy chain and light chain sequences in SEQ ID NO: 27 and SEQ ID NO: 23, respectively, including post-translational modifications of those sequences.

In a further aspect, the invention provides an antibody or immunoconjugate that binds to the same epitope as an anti-CD22 antibody provided herein. For example, in certain embodiments, an antibody or immunoconjugate is provided that binds to the same epitope as an anti-CD22 antibody comprising a VH sequence of SEQ ID NO: 7 and a VL sequence of SEQ ID NO: 8. In certain embodiments, an antibody is provided that binds to an epitope of SEQ ID NO: 28 from, within, or overlapping amino acids 20 to 240.

In a further aspect of the invention, an anti-CD22 antibody according to any of the above embodiments is a monoclonal antibody, including a chimeric, humanized or human antibody. In one embodiment, an anti-CD22 antibody is an antibody fragment, e.g., a Fv, Fab, Fab′, scFv, diabody, or F(ab′)₂ fragment. In another embodiment, the antibody is a substantially full length antibody, e.g., an IgG1 antibody or other antibody class or isotype as defined herein.

In any of the immunoconjugates described above, the antibody may be conjugated to a drug moiety. In some embodiments, the antibody is conjugated to a cytotoxic agent. In some such embodiments, the cytotoxic agent is a pyrrolobenzodiazepine (PBD), such as a PBD dimer. Various nonlimiting exemplary PBD dimers are discussed herein.

In a further aspect, an anti-CD22 antibody or immunoconjugate according to any of the above embodiments may incorporate any of the features, singly or in combination, as described in Sections 1-7 below.

### 1. Antibody Affinity

In certain embodiments, an antibody provided herein has a dissociation constant (Kd) of ≦100 nM, ≦10 nM, ≦1 nM, ≦0.1 nM, ≦0.01 nM, or ≦0.001 nM, and optionally is ≧10⁻¹³ M. (e.g. 10⁻⁸M or less, e.g. from 10⁻⁸M to 10⁻¹³M, e.g., from 10⁻⁹M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at −10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody.

Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for CD22 and the other is for any other antigen. In certain embodiments, bispecific antibodies may bind to two different epitopes of CD22. Bispecific antibodies may also be used to localize cytotoxic agents to cells which express CD22. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to CD22 as well as another, different antigen (see, US 2008/0069820, for example).

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγR¹, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

### d) Cysteine Engineered Antibody Variants

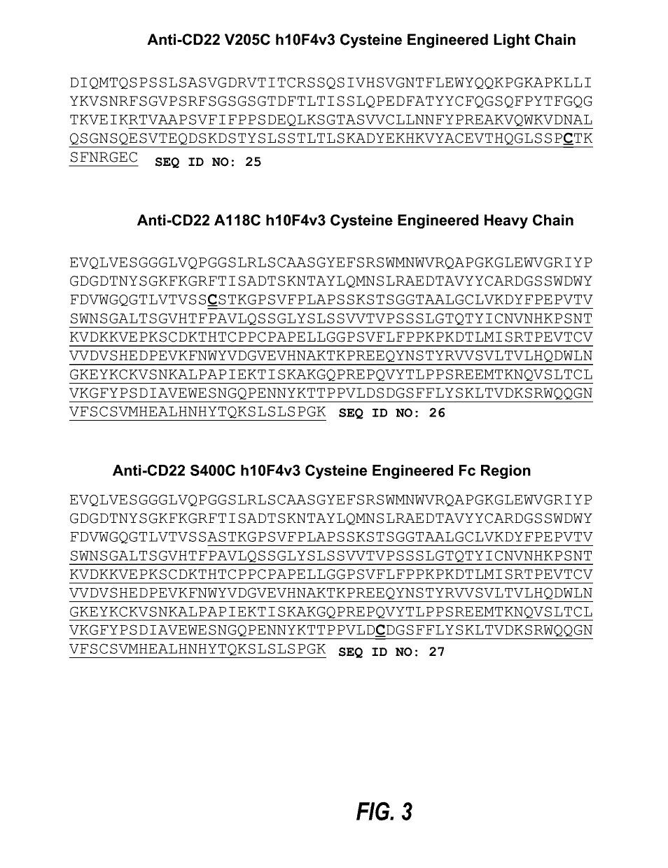

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Nonlimiting exemplary cysteine engineered heavy chains and light chains of anti-CD22 antibodies are shown in FIG. 3 (SEQ ID NOs: 25 to 27). Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

Antibodies may be produced using recombinant methods and compositions, e.g., as described in U.S. Pat. No. 4,816,567. In one embodiment, isolated nucleic acid encoding an anti-CD22 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CD22 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD22 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### C. Assays

Anti-CD22 antibodies provided herein may be identified, screened for, or characterized for their physical/chemical properties and/or biological activities by various assays known in the art.

In one aspect, an antibody is tested for its antigen binding activity, e.g., by known methods such as ELISA, BIACore®, FACS, or Western blot.

In another aspect, competition assays may be used to identify an antibody that competes with any of the antibodies described herein for binding to CD22. In certain embodiments, such a competing antibody binds to the same epitope (e.g., a linear or a conformational epitope) that is bound by an antibody described herein. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized CD22 is incubated in a solution comprising a first labeled antibody that binds to CD22 (e.g., any of the antibodies described herein) and a second unlabeled antibody that is being tested for its ability to compete with the first antibody for binding to CD22. The second antibody may be present in a hybridoma supernatant. As a control, immobilized CD22 is incubated in a solution comprising the first labeled antibody but not the second unlabeled antibody. After incubation under conditions permissive for binding of the first antibody to CD22, excess unbound antibody is removed, and the amount of label associated with immobilized CD22 is measured. If the amount of label associated with immobilized CD22 is substantially reduced in the test sample relative to the control sample, then that indicates that the second antibody is competing with the first antibody for binding to CD22. See Harlow and Lane (1988) Antibodies: A Laboratory Manual ch. 14 (Cold Spring Harbor Laboratory, Cold Spring Harbor, N.Y.).

### D. Immunoconjugates

The invention also provides immunoconjugates comprising an anti-CD22 antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes (i.e., a radio conjugate).

Immunoconjugates allow for the targeted delivery of a drug moiety to a tumor, and, in some embodiments intracellular accumulation therein, where systemic administration of unconjugated drugs may result in unacceptable levels of toxicity to normal cells (Polakis P. (2005) Current Opinion in Pharmacology 5:382-387).

Antibody-drug conjugates (ADC) are targeted chemotherapeutic molecules which combine properties of both antibodies and cytotoxic drugs by targeting potent cytotoxic drugs to antigen-expressing tumor cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chan, R. V. (2008) Acc. Chem. Res. 41:98-107.

The ADC compounds of the invention include those with anticancer activity. In some embodiments, the ADC compounds include an antibody conjugated, i.e. covalently attached, to the drug moiety. In some embodiments, the antibody is covalently attached to the drug moiety through a linker. The antibody-drug conjugates (ADC) of the invention selectively deliver an effective dose of a drug to tumor tissue whereby greater selectivity, i.e. a lower efficacious dose, may be achieved while increasing the therapeutic index (“therapeutic window”).

The drug moiety (D) of the antibody-drug conjugates (ADC) may include any compound, moiety or group that has a cytotoxic or cytostatic effect. Exemplary drug moieties include, but are not limited to, pyrrolobenzodiazepine (PBD) and derivatives thereof that have cytotoxic activity. Nonlimiting examples of such immunoconjugates are discussed in further detail below.

1. Exemplary Antibody-Drug Conjugates

An exemplary embodiment of an antibody-drug conjugate (ADC) compound comprises an antibody (Ab) which targets a tumor cell, a drug moiety (D), and a linker moiety (L) that attaches Ab to D. In some embodiments, the antibody is attached to the linker moiety (L) through one or more amino acid residues, such as lysine and/or cysteine.

An exemplary ADC has Formula I:

Ab-(L-D)ₚ I

where p is 1 to about 20. In some embodiments, the number of drug moieties that can be conjugated to an antibody is limited by the number of free cysteine residues. In some embodiments, free cysteine residues are introduced into the antibody amino acid sequence by the methods described herein. Exemplary ADC of Formula I include, but are not limited to, antibodies that have 1, 2, 3, or 4 engineered cysteine amino acids (Lyon, R. et al (2012) Methods in Enzym. 502:123-138). In some embodiments, one or more free cysteine residues are already present in an antibody, without the use of engineering, in which case the existing free cysteine residues may be used to conjugate the antibody to a drug. In some embodiments, an antibody is exposed to reducing conditions prior to conjugation of the antibody in order to generate one or more free cysteine residues.

### a) Exemplary Linkers

A “Linker” (L) is a bifunctional or multifunctional moiety that can be used to link one or more drug moieties (D) to an antibody (Ab) to form an antibody-drug conjugate (ADC) of Formula I. In some embodiments, antibody-drug conjugates (ADC) can be prepared using a Linker having reactive functionalities for covalently attaching to the drug and to the antibody. For example, in some embodiments, a cysteine thiol of an antibody (Ab) can form a bond with a reactive functional group of a linker or a drug-linker intermediate to make an ADC.

In one aspect, a linker has a functionality that is capable of reacting with a free cysteine present on an antibody to form a covalent bond. Nonlimiting exemplary such reactive functionalities include maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See, e.g., the conjugation method at page 766 of Klussman, et al (2004), Bioconjugate Chemistry 15(4):765-773, and the Examples herein.

In some embodiments, a linker has a functionality that is capable of reacting with an electrophilic group present on an antibody. Exemplary such electrophilic groups include, but are not limited to, aldehyde and ketone carbonyl groups. In some embodiments, a heteroatom of the reactive functionality of the linker can react with an electrophilic group on an antibody and form a covalent bond to an antibody unit. Nonlimiting exemplary such reactive functionalities include, but are not limited to, hydrazide, oxime, amino, hydrazine, thiosemicarbazone, hydrazine carboxylate, and arylhydrazide.

A linker may comprise one or more linker components. Exemplary linker components include 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N—Succinimidyl 4-(2-pyridylthio) pentanoate (“SPP”), and 4-(N-maleimidomethyl) cyclohexane-1 carboxylate (“MCC”). Various linker components are known in the art, some of which are described below.

A linker may be a “cleavable linker,” facilitating release of a drug. Nonlimiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992); U.S. Pat. No. 5,208,020).

In certain embodiments, a linker has the following Formula II:

-Aₐ-W_{w}—Y_{y—} II

wherein A is a “stretcher unit”, and a is an integer from 0 to 1; W is an “amino acid unit”, and w is an integer from 0 to 12; Y is a “spacer unit”, and y is 0, 1, or 2. An ADC comprising the linker of Formula II has the Formula I(A): Ab-(Aₐ-W_{w}—Y_{y}-D)p, wherein Ab, D, and p are defined as above for Formula I. Exemplary embodiments of such linkers are described in U.S. Pat. No. 7,498,298, which is expressly incorporated herein by reference.

In some embodiments, a linker component comprises a “stretcher unit” (A) that links an antibody to another linker component or to a drug moiety. Nonlimiting exemplary stretcher units are shown below (wherein the wavy line indicates sites of covalent attachment to an antibody, drug, or additional linker components):

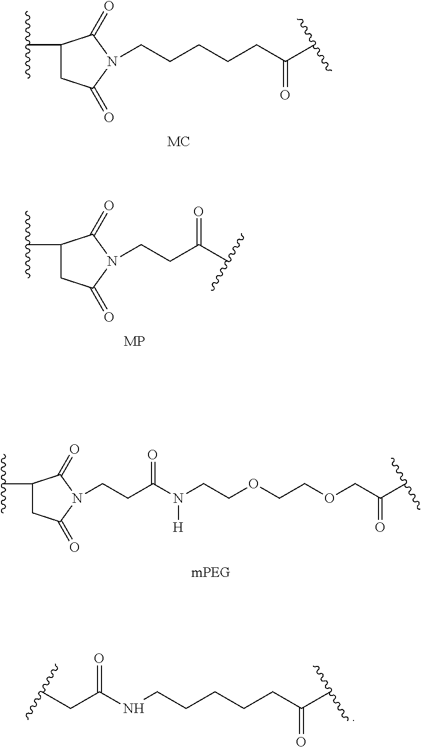 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O ; "."]

In some embodiments, a linker component comprises an “amino acid unit” (W). In some such embodiments, the amino acid unit allows for cleavage of the linker by a protease, thereby facilitating release of the drug from the immunoconjugate upon exposure to intracellular proteases, such as lysosomal enzymes (Doronina et al. (2003) Nat. Biotechnol. 21:778-784). Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline. Amino acid units can be designed and optimized for enzymatic cleavage by a particular enzyme, for example, a tumor-associated protease, cathepsin B, C and D, or a plasmin protease.

Typically, peptide-type linkers can be prepared by forming a peptide bond between two or more amino acids and/or peptide fragments. Such peptide bonds can be prepared, for example, according to a liquid phase synthesis method (e.g., E. Schröder and K. Lübke (1965) “The Peptides”, volume 1, pp 76-136, Academic Press).

In some embodiments, a linker component comprises a “spacer” unit that links the antibody to a drug moiety, either directly or through a stretcher unit and/or an amino acid unit. A spacer unit may be “self-immolative” or a “non-self-immolative.” A “non-self-immolative” spacer unit is one in which part or all of the spacer unit remains bound to the drug moiety upon cleavage of the ADC. Examples of non-self-immolative spacer units include, but are not limited to, a glycine spacer unit and a glycine-glycine spacer unit. In some embodiments, enzymatic cleavage of an ADC containing a glycine-glycine spacer unit by a tumor-cell associated protease results in release of a glycine-glycine-drug moiety from the remainder of the ADC. In some such embodiments, the glycine-glycine-drug moiety is subjected to a hydrolysis step in the tumor cell, thus cleaving the glycine-glycine spacer unit from the drug moiety.

A “self-immolative” spacer unit allows for release of the drug moiety. In certain embodiments, a spacer unit of a linker comprises a p-aminobenzyl unit. In some such embodiments, a p-aminobenzyl alcohol is attached to an amino acid unit via an amide bond, and a carbamate, methylcarbamate, or carbonate is made between the benzyl alcohol and the drug (Hamann et al. (2005) Expert Opin. Ther. Patents (2005) 15:1087-1103). In some embodiments, the spacer unit comprises p-aminobenzyloxycarbonyl (PAB). In some embodiments, an ADC comprising a self-immolative linker has the structure:

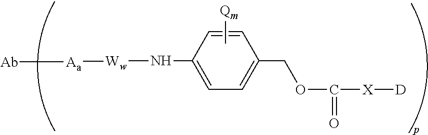

wherein Q is —C₁-C₈ alkyl, —O—(C₁-C₈ alkyl), -halogen, -nitro, or -cyano; m is an integer ranging from 0 to 4; X may be one or more additional spacer units or may be absent; and p ranges from 1 to about 20. In some embodiments, p ranges from 1 to 10, 1 to 7, 1 to 5, or 1 to 4. Nonlimiting exemplary X spacer units include:

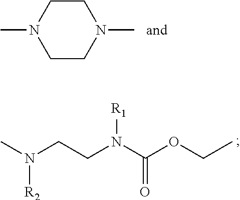

wherein R₁ and R₂ are independently selected from H and C₁-C₆ alkyl. In some embodiments, R1 and R2 are each —CH₃.

Other examples of self-immolative spacers include, but are not limited to, aromatic compounds that are electronically similar to the PAB group, such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Linkage of a drug to the α-carbon of a glycine residue is another example of a self-immolative spacer that may be useful in ADC (Kingsbury et al (1984) J. Med. Chem. 27:1447).

In some embodiments, linker L may be a dendritic type linker for covalent attachment of more than one drug moiety to an antibody through a branching, multifunctional linker moiety (Sun et al (2002) Bioorganic & Medicinal Chemistry Letters 12:2213-2215; Sun et al (2003) Bioorganic & Medicinal Chemistry 11:1761-1768). Dendritic linkers can increase the molar ratio of drug to antibody, i.e. loading, which is related to the potency of the ADC. Thus, where an antibody bears only one reactive cysteine thiol group, a multitude of drug moieties may be attached through a dendritic linker.

Nonlimiting exemplary linkers are shown below in the context of an ADC of Formula I:

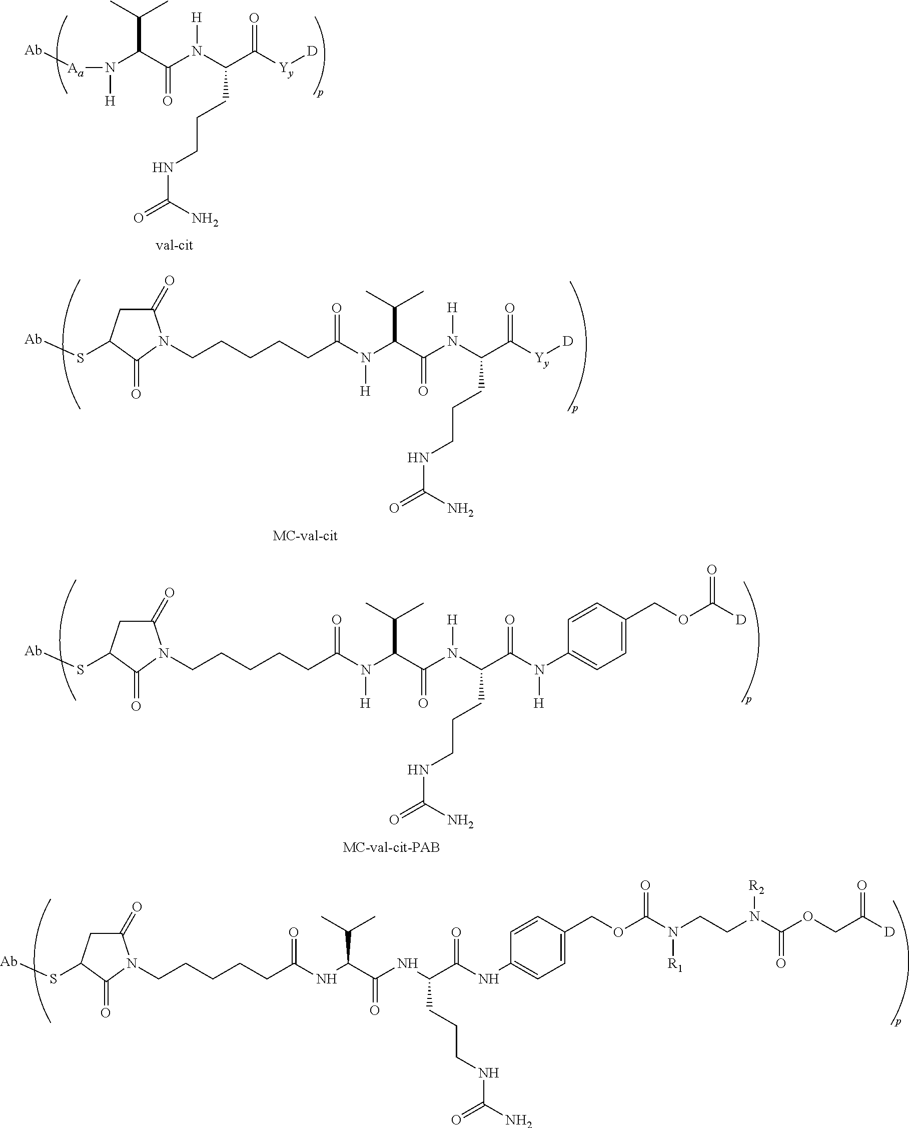

wherein R₁ and R₂ are independently selected from H and C₁-C₆ alkyl. In some embodiments, R1 and R2 are each —CH₃.

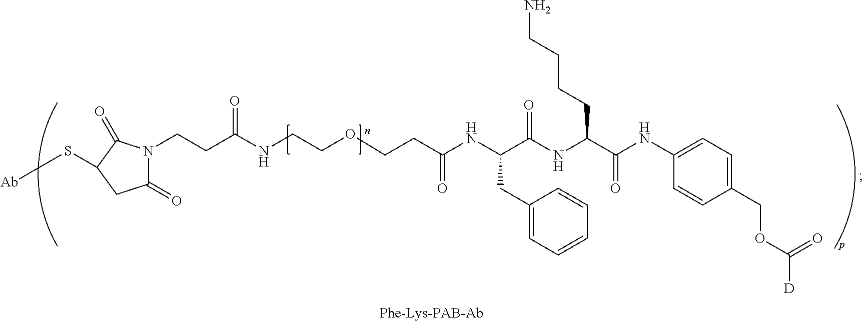

wherein n is 0 to 12. In some embodiments, n is 2 to 10. In some embodiments, n is 4 to 8.

Further nonlimiting exemplary ADCs include the structures:

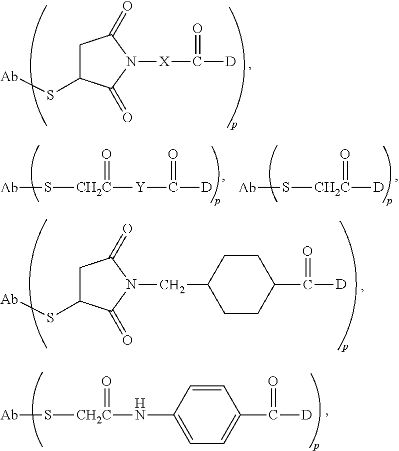

where X is:

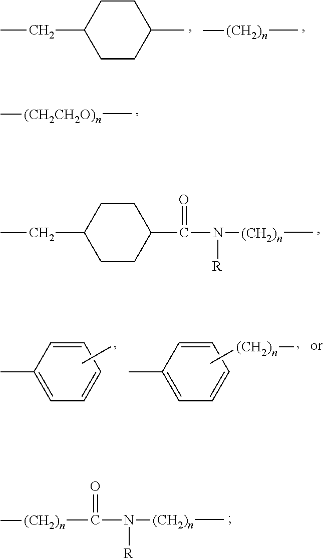

Y is:

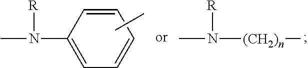

each R is independently H or C₁-C₆ alkyl; and n is 1 to 12.

In some embodiments, a linker is substituted with groups that modulate solubility and/or reactivity. As a nonlimiting example, a charged substituent such as sulfonate (—SO₃⁻) or ammonium may increase water solubility of the linker reagent and facilitate the coupling reaction of the linker reagent with the antibody and/or the drug moiety, or facilitate the coupling reaction of Ab-L (antibody-linker intermediate) with D, or D-L (drug-linker intermediate) with Ab, depending on the synthetic route employed to prepare the ADC. In some embodiments, a portion of the linker is coupled to the antibody and a portion of the linker is coupled to the drug, and then the Ab-(linker portion)^{a} is coupled to drug-(linker portion)^{b} to form the ADC of Formula I.

The compounds of the invention expressly contemplate, but are not limited to, ADC prepared with the following linker reagents: bis-maleimido-trioxyethylene glycol (BMPEO), N-(β-maleimidopropyloxy)-N-hydroxy succinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(beta-maleimidopropionamido)hexanoate] (SMPH), iminothiolane (IT), sulfo-EMCS, sulfo-GMBS, sulfo-KMUS, sulfo-MBS, sulfo-SIAB, sulfo-SMCC, and sulfo-SMPB, and succinimidyl-(4-vinylsulfone)benzoate (SVSB), and including bis-maleimide reagents: dithiobismaleimidoethane (DTME), 1,4-Bismaleimidobutane (BMB), 1,4 Bismaleimidyl-2,3-dihydroxybutane (BMDB), bismaleimidohexane (BMH), bismaleimidoethane (BMOE), BM(PEG)₂ (shown below), and BM(PEG)₃ (shown below); bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In some embodiments, bis-maleimide reagents allow the attachment of the thiol group of a cysteine in the antibody to a thiol-containing drug moiety, linker, or linker-drug intermediate. Other functional groups that are reactive with thiol groups include, but are not limited to, iodoacetamide, bromoacetamide, vinyl pyridine, disulfide, pyridyl disulfide, isocyanate, and isothiocyanate.

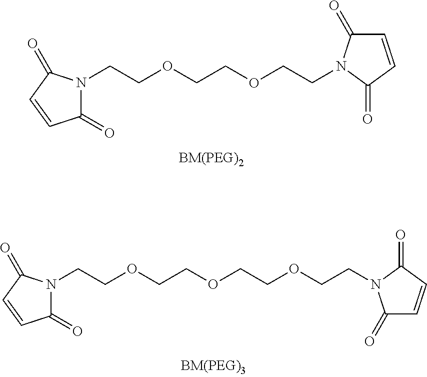 [SMILES: O=C1C=CC(=O)N1CCOCCOCCN1C(=O)C=CC1=O ; O=C1C=CC(=O)N1CCOCCOCCOCCN1C(=O)C=CC1=O]

Certain useful linker reagents can be obtained from various commercial sources, such as Pierce Biotechnology, Inc. (Rockford, Ill.), Molecular Biosciences Inc. (Boulder, Colo.), or synthesized in accordance with procedures described in the art; for example, in Toki et al (2002) J. Org. Chem. 67:1866-1872; Dubowchik, et al. (1997) Tetrahedron Letters, 38:5257-60; Walker, M. A. (1995) J. Org. Chem. 60:5352-5355; Frisch et al (1996) Bioconjugate Chem. 7:180-186; U.S. Pat. No. 6,214,345; WO 02/088172; US 2003130189; US2003096743; WO 03/026577; WO 03/043583; and WO 04/032828.

Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO94/11026.

### b) Exemplary Drug Moieties

In some embodiments, an ADC comprises a pyrrolobenzodiazepine (PBD). In some embodiments, PBD dimers recognize and bind to specific DNA sequences. The natural product anthramycin, a PBD, was first reported in 1965 (Leimgruber, et al., (1965) J. Am. Chem. Soc., 87:5793-5795; Leimgruber, et al., (1965) J. Am. Chem. Soc., 87:5791-5793). Since then, a number of PBDs, both naturally-occurring and analogues, have been reported (Thurston, et al., (1994) Chem. Rev. 1994, 433-465 including dimers of the tricyclic PBD scaffold (U.S. Pat. No. 6,884,799; U.S. Pat. No. 7,049,311; U.S. Pat. No. 7,067,511; U.S. Pat. No. 7,265,105; U.S. Pat. No. 7,511,032; U.S. Pat. No. 7,528,126; U.S. Pat. No. 7,557,099). Without intending to be bound by any particular theory, it is believed that the dimer structure imparts the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, (1986) Acc. Chem. Res., 19:230-237). Dimeric PBD compounds bearing C2 aryl substituents have been shown to be useful as cytotoxic agents (Hartley et al (2010) Cancer Res. 70(17):6849-6858; Antonow (2010) J. Med. Chem. 53(7):2927-2941; Howard et al (2009) Bioorganic and Med. Chem. Letters 19(22):6463-6466).

PBD dimers have been conjugated to antibodies and the resulting ADC shown to have anti-cancer properties. Nonlimiting exemplary linkage sites on the PBD dimer include the five-membered pyrrolo ring, the tether between the PBD units, and the N10-C11 imine group (WO 2009/016516; US 2009/304710; US 2010/047257; US 2009/036431; US 2011/0256157; WO 2011/130598).

Nonlimiting exemplary PBD dimer components of ADCs are of Formula A:

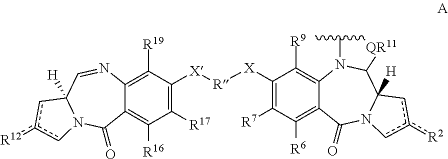

and salts and solvates thereof, wherein:

the wavy line indicates the covalent attachment site to the linker;

the dotted lines indicate the optional presence of a double bond between C1 and C2 or C2 and C3;

R² is independently selected from H, OH, ═O, ═CH₂, CN, R, OR, ═CH—R^{D}, ═C(R^{D})₂, O—SO₂—R, CO₂R and COR, and optionally further selected from halo or dihalo, wherein R^{D} is independently selected from R, CO₂R, COR, CHO, CO₂H, and halo;

R⁶ and R⁹ are independently selected from H, R, OH, OR, SH, SR, NH₂, NHR, NRR′, NO₂, Me₃Sn and halo;

R⁷ is independently selected from H, R, OH, OR, SH, SR, NH₂, NHR, NRR′, NO₂, Me₃Sn and halo;

Q is independently selected from O, S and NH;

R¹¹ is either H, or R or, where Q is O, SO₃M, where M is a metal cation;

R and R′ are each independently selected from optionally substituted C₁₋₁₂ alkyl, C₃₋₂₀ heterocyclyl and C₅₋₂₀ aryl groups, and optionally in relation to the group NRR′, R and R′ together with the nitrogen atom to which they are attached form an optionally substituted 4-, 5-, 6- or 7-membered heterocyclic ring;

R¹², R¹⁶, R¹⁹ and R¹⁷ are as defined for R², R⁶, R⁹ and R⁷ respectively;

R″ is a C₃₋₁₂ alkylene group, which chain may be interrupted by one or more heteroatoms, e.g. O, S, N(H), NMe and/or aromatic rings, e.g. benzene or pyridine, which rings are optionally substituted; and

X and X′ are independently selected from O, S and N(H).

In some embodiments, R⁹ and R¹⁹ are H.

In some embodiments, R⁶ and R¹⁶ are H.

In some embodiments, R⁷ are R¹⁷ are both OR^{7A}, where R^{7A} is optionally substituted C₁₋₄ alkyl. In some embodiments, R^{7A} is Me. In some embodiments, R^{7A} is CH₂Ph, where Ph is a phenyl group.

In some embodiments, X is O.

In some embodiments, R¹¹ is H.

In some embodiments, there is a double bond between C2 and C3 in each monomer unit.

In some embodiments, R² and R¹² are independently selected from H and R. In some embodiments, R² and R¹² are independently R. In some embodiments, R² and R¹² are independently optionally substituted C₅₋₂₀ aryl or C₅₋₇ aryl or C₈₋₁₀ aryl. In some embodiments, R² and R¹² are independently optionally substituted phenyl, thienyl, napthyl, pyridyl, quinolinyl, or isoquinolinyl. In some embodiments, R² and R¹² are independently selected from ═O, ═CH₂, ═CH—R^{D}, and ═C(R^{D})₂. In some embodiments, R² and R¹² are each ═CH₂. In some embodiments, R² and R¹² are each H. In some embodiments, R² and R¹² are each ═O. In some embodiments, R² and R¹² are each ═CF₂. In some embodiments, R² and/or R¹² are independently ═C(R^{D})₂. In some embodiments, R² and/or R¹² are independently ═CH—R^{D}.

In some embodiments, when R² and/or R¹² is ═CH—R^{D}, each group may independently have either configuration shown below:

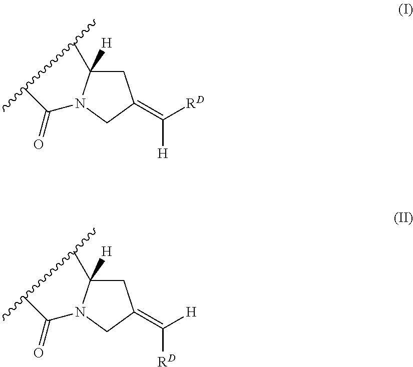

In some embodiments, a ═CH—R^{D} is in configuration (I).

In some embodiments, R″ is a C₃ alkylene group or a C₅ alkylene group.

In some embodiments, an exemplary PBD dimer component of an ADC has the structure of Formula A(I):

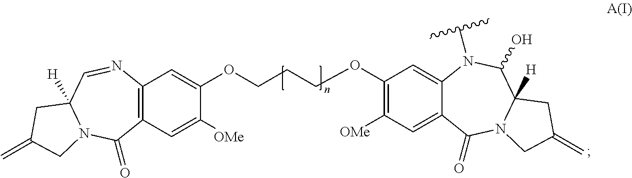

wherein n is 0 or 1.

In some embodiments, an exemplary PBD dimer component of an ADC has the structure of Formula A(II):

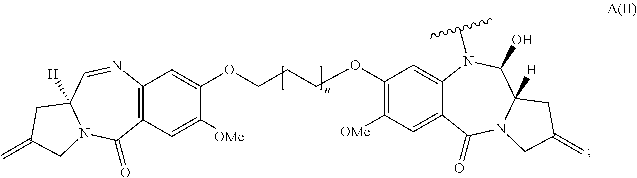

wherein n is 0 or 1.

In some embodiments, an exemplary PBD dimer component of an ADC has the structure of Formula A(III):

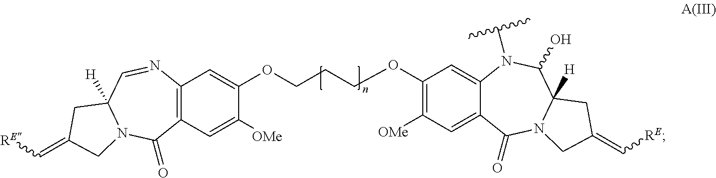

wherein R^{E} and R^{E″} are each independently selected from H or R^{D}, wherein R^{D} is defined as above; and
wherein n is 0 or 1.

In some embodiments, n is 0. In some embodiments, n is 1. In some embodiments, R^{E} and/or R^{E″} is H. In some embodiments, R^{E} and R^{E″} are H. In some embodiments, R^{E} and/or R^{E″} is R^{D}, wherein R^{D} is optionally substituted C₁₋₁₂ alkyl. In some embodiments, R^{E} and/or R^{E″} is R^{D}, wherein R^{D} is methyl.

In some embodiments, an exemplary PBD dimer component of an ADC has the structure of Formula A(IV):

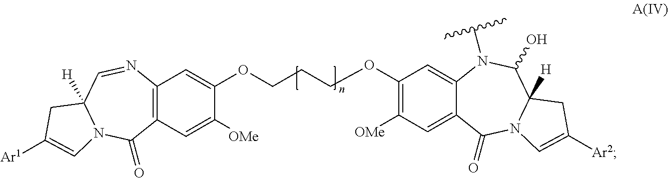

wherein Ar¹ and Ar² are each independently optionally substituted C₅₋₂₀ aryl; wherein Ar¹ and Ar² may be the same or different; and
wherein n is 0 or 1.

In some embodiments, an exemplary PBD dimer component of an ADC has the structure of Formula A(V):

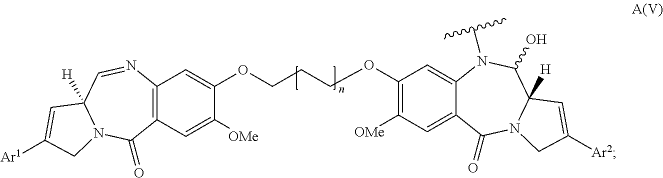

wherein Ar¹ and Ar² are each independently optionally substituted C₅₋₂₀ aryl; wherein Ar¹ and Ar² may be the same or different; and

wherein n is 0 or 1.

In some embodiments, Ar¹ and Ar² are each independently selected from optionally substituted phenyl, furanyl, thiophenyl and pyridyl. In some embodiments, Ar¹ and Ar² are each independently optionally substituted phenyl. In some embodiments, Ar¹ and Ar² are each independently optionally substituted thien-2-yl or thien-3-yl. In some embodiments, Ar¹ and Ar² are each independently optionally substituted quinolinyl or isoquinolinyl. The quinolinyl or isoquinolinyl group may be bound to the PBD core through any available ring position. For example, the quinolinyl may be quinolin-2-yl, quinolin-3-yl, quinolin-4yl, quinolin-5-yl, quinolin-6-yl, quinolin-7-yl and quinolin-8-yl. In some embodiments, the quinolinyl is selected from quinolin-3-yl and quinolin-6-yl. The isoquinolinyl may be isoquinolin-1-yl, isoquinolin-3-yl, isoquinolin-4yl, isoquinolin-5-yl, isoquinolin-6-yl, isoquinolin-7-yl and isoquinolin-8-yl. In some embodiments, the isoquinolinyl is selected from isoquinolin-3-yl and isoquinolin-6-yl.

Further nonlimiting exemplary PBD dimer components of ADCs are of Formula B:

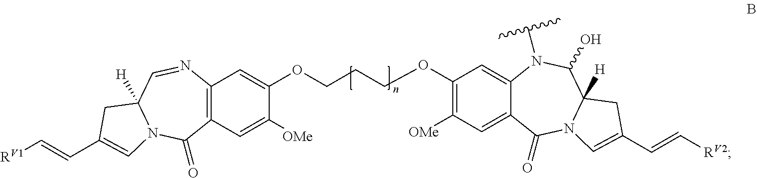

and salts and solvates thereof, wherein:

the wavy line indicates the covalent attachment site to the linker;

the wavy line connected to the OH indicates the S or R configuration;

R^{V1} and R^{V2} are independently selected from H, methyl, ethyl, and phenyl (which phenyl may be optionally substituted with fluoro, particularly in the 4 position) and C₅₋₆ heterocyclyl; wherein R^{V1} and R^{V2} may be the same or different; and

n is 0 or 1.

In some embodiments, R^{V1} and R^{V2} are independently selected from H, phenyl, and 4-fluorophenyl.

In some embodiments, a linker may be attached at one of various sites of the PBD dimer drug moiety, including the N10 imine of the B ring, the C-2 endo/exo position of the C ring, or the tether unit linking the A rings (see structures C(I) and C(II) below).

Nonlimiting exemplary PBD dimer components of ADCs include Formulas C(I) and C(II):

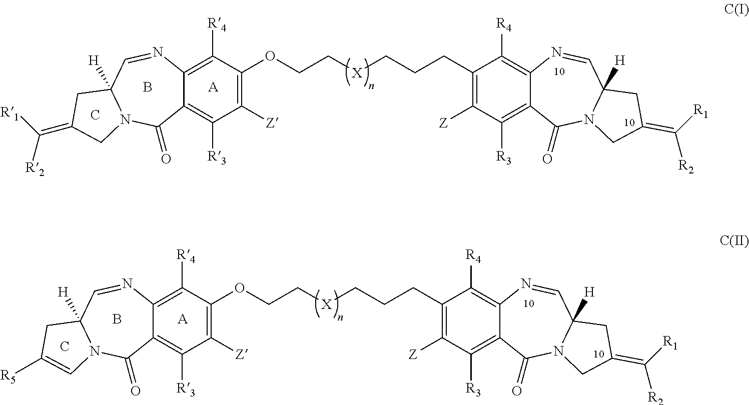

Formulas C(I) and C(II) are shown in their N10-C11 imine form. Exemplary PBD drug moieties also include the carbinolamine and protected carbinolamine forms as well, as shown in the table below:

| | | |
|---|---|---|
| 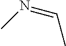 Imine | 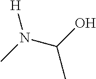 Carbinolamine | 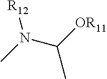 Protected Carbinolamine |

wherein:

X is CH₂ (n=1 to 5), N, or O;

Z and Z′ are independently selected from OR and NR₂, where R is a primary, secondary or tertiary alkyl chain containing 1 to 5 carbon atoms;

R₁, R′₁, R₂ and R′₂ are each independently selected from H, C₁-C₈ alkyl, C₂-C₈ alkenyl, C₂-C₈ alkynyl, C₅₋₂₀ aryl (including substituted aryls), C₅₋₂₀ heteroaryl groups, —NH₂, —NHMe, —OH, and —SH, where, in some embodiments, alkyl, alkenyl and alkynyl chains comprise up to 5 carbon atoms;

R₃ and R′₃ are independently selected from H, OR, NHR, and NR₂, where R is a primary, secondary or tertiary alkyl chain containing 1 to 5 carbon atoms;

R₄ and R′₄ are independently selected from H, Me, and OMe;

R₅ is selected from C₁-C₈ alkyl, C₂-C₈ alkenyl, C₂-C₈ alkynyl, C₅₋₂₀ aryl (including aryls substituted by halo, nitro, cyano, alkoxy, alkyl, heterocyclyl) and C₅₋₂₀ heteroaryl groups, where, in some embodiments, alkyl, alkenyl and alkynyl chains comprise up to 5 carbon atoms;

R₁₁ is H, C₁-C₈ alkyl, or a protecting group (such as acetyl, trifluoroacetyl, t-butoxycarbonyl (BOC), benzyloxycarbonyl (CBZ), 9-fluorenylmethylenoxycarbonyl (Fmoc), or a moiety comprising a self-immolating unit such as valine-citrulline-PAB);

R₁₂ is H, C₁-C₈ alkyl, or a protecting group;

wherein a hydrogen of one of R₁, R′₁, R₂, R′₂, R₅, or R₁₂ or a hydrogen of the —OCH₂CH₂(X)ₙCH₂CH₂O— spacer between the A rings is replaced with a bond connected to the linker of the ADC.

Exemplary PBD dimer portions of ADC include, but are not limited to (the wavy line indicates the site of covalent attachment to the linker):

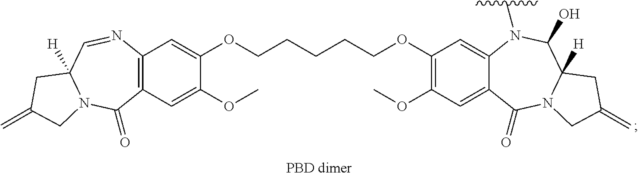

In some embodiments, an antibody-drug conjugate comprising a PBD dimer has the structure of formula (D):

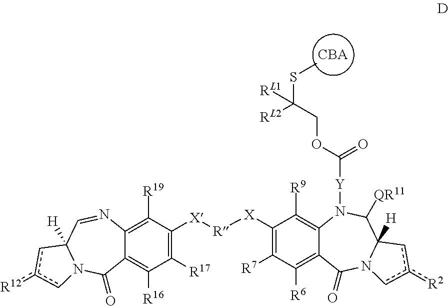

and salts and solvates thereof, wherein:

the dotted lines indicate the optional presence of a double bond between C1 and C2 or C2 and C3;

R² is independently selected from H, OH, ═O, ═CH₂, CN, R, OR, ═CH—R^{D}, ═C(R^{D})₂, O—SO₂—R, CO₂R and COR, and optionally further selected from halo or dihalo; where R_{D} is independently selected from R, CO₂R, COR, CHO, CO₂H, and halo;

R⁶ and R⁹ are independently selected from H, R, OH, OR, SH, SR, NH₂, NHR, NRR′, NO₂, Me₃Sn and halo;

R⁷ is independently selected from H, R, OH, OR, SH, SR, NH₂, NHR, NRR′, NO₂, Me₃Sn and halo;

Y is selected from a single bond, and a group of formulae a1 or a2:

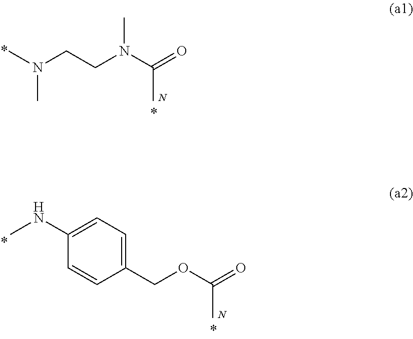

where N shows where the group binds to the N10 of the PBD moiety;

R^{L1} and R^{L2} are independently selected from H and methyl, or together with the carbon atom to which they are bound form a cyclopropylene group;

CBA represents the antibody;

Q is independently selected from O, S and NH;

R¹¹ is either H, or R or, where Q is O, SO₃M, where M is a metal cation;

R and R′ are each independently selected from optionally substituted C₁₋₁₂ alkyl, C₃₋₂₀ heterocyclyl and C₅₋₂₀ aryl groups, and optionally in relation to the group NRR′, R and R′ together with the nitrogen atom to which they are attached form an optionally substituted 4-, 5-, 6- or 7-membered heterocyclic ring;

wherein R¹², R¹⁶, R¹⁹ and R¹⁷ are as defined for R², R⁶, R⁹ and R⁷ respectively;

wherein R″ is a C₃₋₁₂ alkylene group, which chain may be interrupted by one or more heteroatoms, e.g. O, S, N(H), NMe and/or aromatic rings, e.g. benzene or pyridine, which rings are optionally substituted;

X and X′ are independently selected from O, S and N(H).

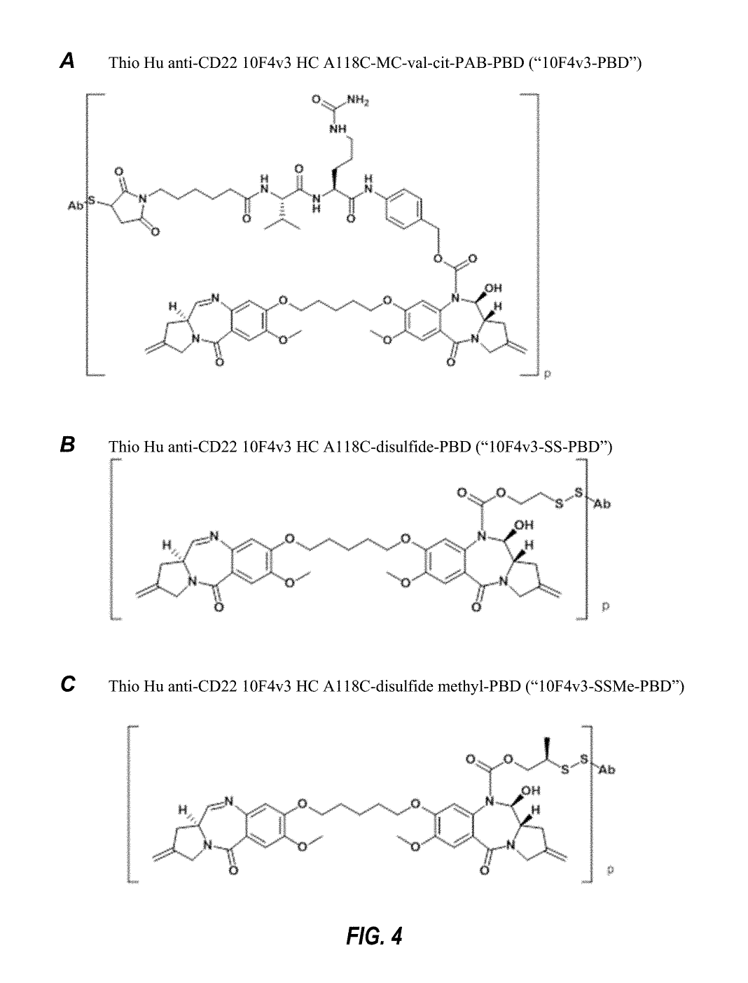

In some embodiments, the antibody is linked to the PBD dimer through a cysteine to form a disulfide linkage, which is shown, e.g., in FIGS. 4B and 4C.

In some embodiments, formula D is selected from the following formulae D-I, D-II and D-III, depending on Y:

| Y | | D | |
|---|---|---|---|
| Single bond | | 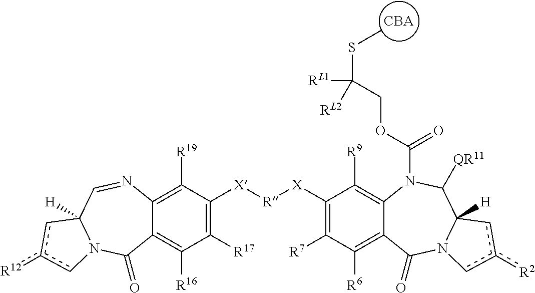 | D-I |
| 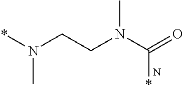 | (a1) | 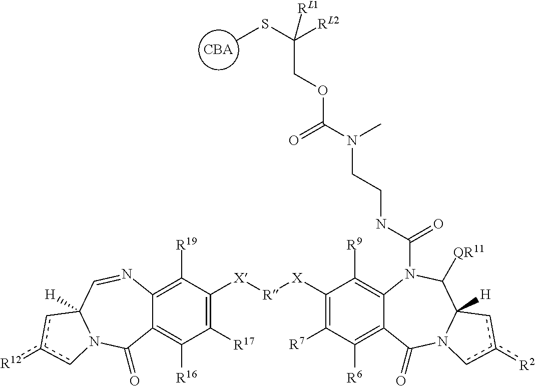 | D-II |
| 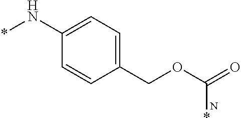 | (a2) | 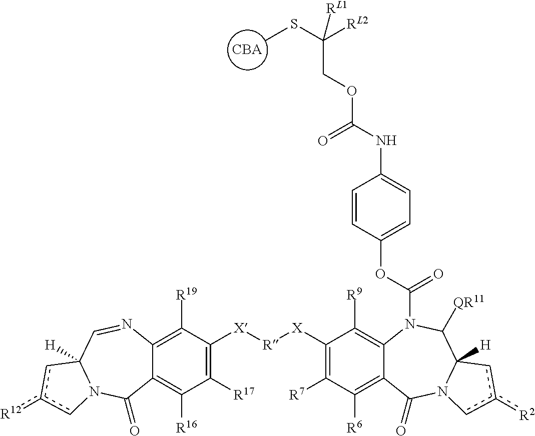 | D-III |

In compounds of formula A:

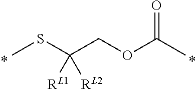

is the sulfur linking group.

In some embodiments, the ADC comprises the structure:

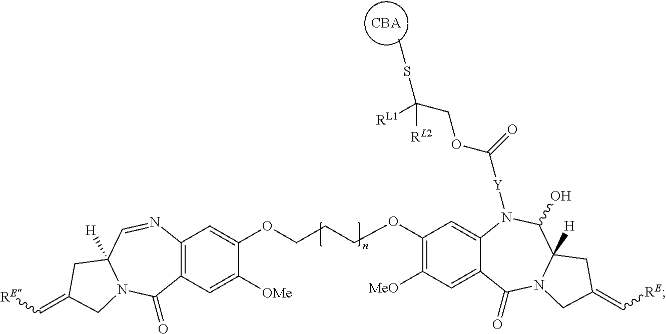

and in some embodiments, the ADC comprises the structure:

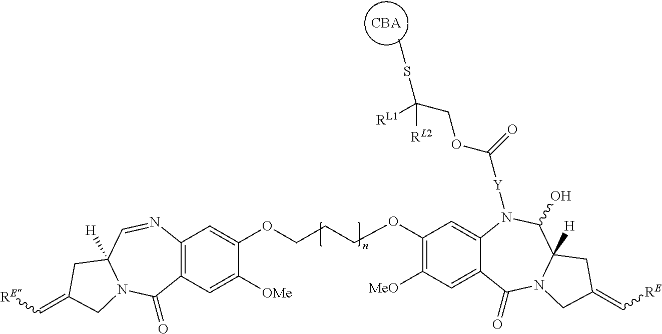

wherein CBA is the antibody, and n is 0 or 1. Y, R^{L1} and R^{L2} are as previously defined, and R^{E} and R^{E″} are each independently selected from H or R^{D}.

In any of the embodiments described above, certain substituents may be defined as follows, where appropriate:

n is 0;

n is 1;

R^{E} is H;

R^{E} is R^{D}, where R^{D} is optionally substituted alkyl;

R^{E} is R^{D}, where R^{D} is methyl;

R^{L1} and R^{L2} are H;

R^{L1} and R^{L2} are Me.

In some embodiments, the ADC comprises the structure:

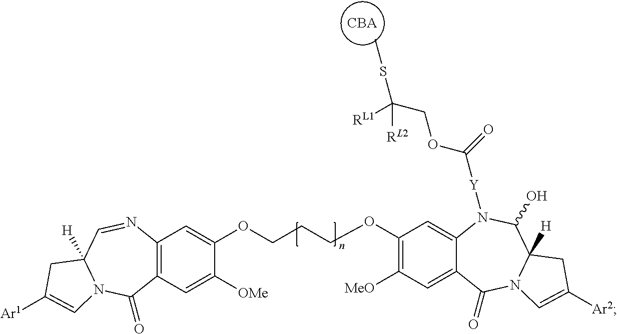

and in some embodiments, the ADC comprises the structure:

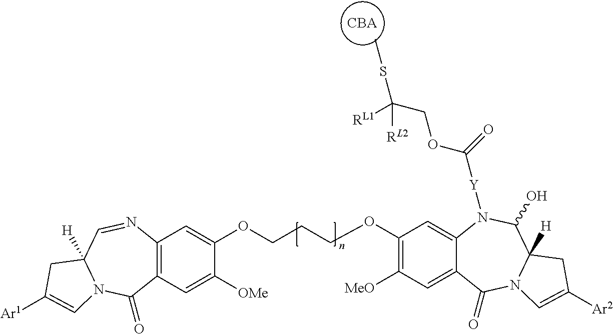

wherein CBA is the antibody, and n is 0 or 1. Y, R^{L1} and R^{L2} are as previously defined, and Ar¹ and Ar² are each independently optionally substituted C₅₋₂₀ aryl. Ar¹ and Ar² may be the same or different.

In some embodiments, Ar¹ and Ar² are each independently selected from optionally substituted phenyl, furanyl, thiophenyl and pyridyl. In some embodiments, Ar¹ and Ar² are each optionally substituted phenyl. In some embodiments, Ar¹ and Ar² are each optionally substituted thien-2-yl or thien-3-yl. In some embodiments, Ar¹ and Ar² are each optionally substituted quinolinyl or isoquinolinyl.

In various embodiments, the quinolinyl or isoquinolinyl group may be bound to the PBD core through any available ring position. For example, the quinolinyl may be quinolin-2-yl, quinolin-3-yl, quinolin-4yl, quinolin-5-yl, quinolin-6-yl, quinolin-7-yl and quinolin-8-yl. Of these quinolin-3-yl and quinolin-6-yl may be preferred. The isoquinolinyl may be isoquinolin-1-yl, isoquinolin-3-yl, isoquinolin-4yl, isoquinolin-5-yl, isoquinolin-6-yl, isoquinolin-7-yl and isoquinolin-8-yl. Of these isoquinolin-3-yl and isoquinolin-6-yl may be preferred.

In some embodiments, the ADC comprises the structure:

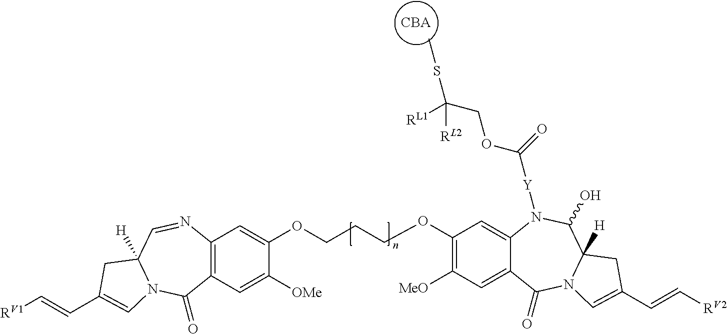

and in some embodiments, the ADC comprises the structure:

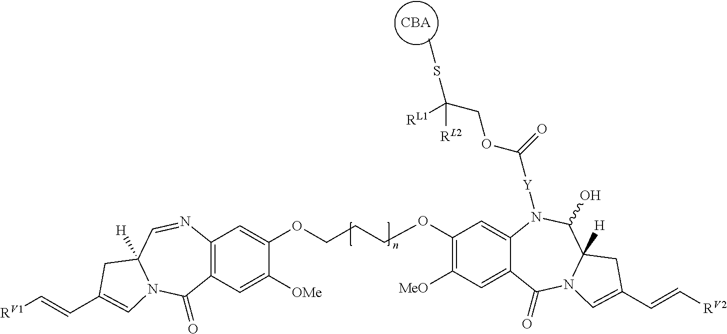

wherein CBA is the antibody, and n is 0 or 1. Y, R^{L1} and R^{L2} are as previously defined, and R^{V1} and R^{V2} are independently selected from H, methyl, ethyl and phenyl (which phenyl may be optionally substituted with fluoro, in some embodiments, in the 4 position) and C₅₋₆ heterocyclyl. R^{V1} and R^{V2} may y be the same or different. In some embodiments, R^{V1} and R^{V2} may be independently selected from H, phenyl, and 4-fluorophenyl.

Nonlimiting exemplary embodiments of ADCs comprising PBD dimers have the following structures:

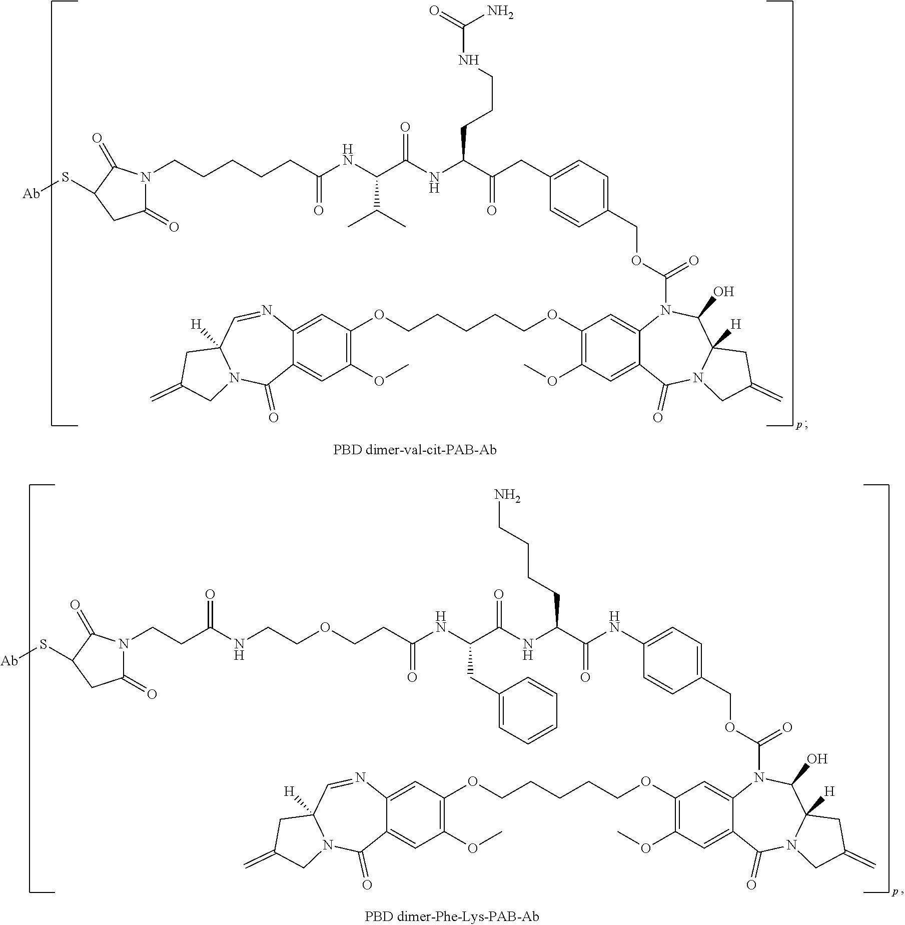

wherein:

n is 0 to 12. In some embodiments, n is 2 to 10. In some embodiments, n is 4 to 8. In some embodiments, n is selected from 4, 5, 6, 7, and 8.

The linkers of PBD dimer-val-cit-PAB-Ab and the PBD dimer-Phe-Lys-PAB-Ab are protease cleavable.

Nonlimiting exemplary embodiments of ADCs comprising PBD dimers have the following structures:

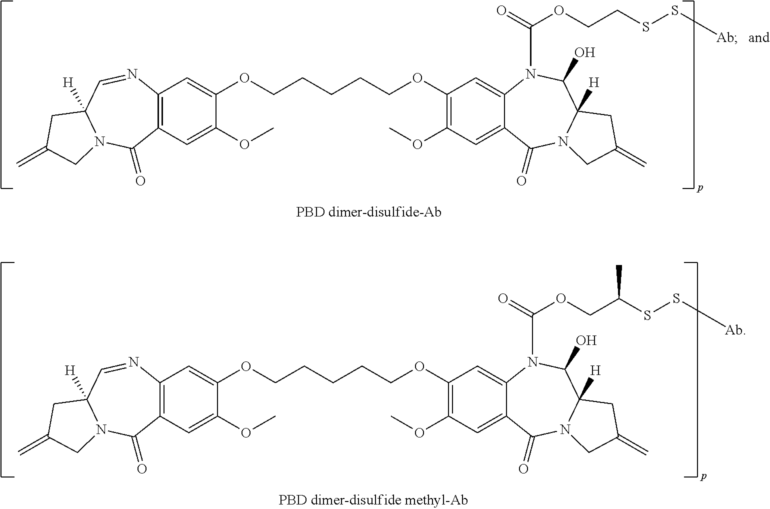

PBD dimers and ADC comprising PBD dimers may be prepared according to methods known in the art, and methods described herein. See, e.g., WO 2009/016516; US 2009/304710; US 2010/047257; US 2009/036431; US 2011/0256157; WO 2011/130598.

### c) Drug Loading

Drug loading is represented by p, the average number of drug moieties per antibody in a molecule of Formula I. Drug loading may range from 1 to 20 drug moieties (D) per antibody. ADCs of Formula I include collections of antibodies conjugated with a range of drug moieties, from 1 to 20. The average number of drug moieties per antibody in preparations of ADC from conjugation reactions may be characterized by conventional means such as mass spectroscopy, ELISA assay, and HPLC. The quantitative distribution of ADC in terms of p may also be determined. In some instances, separation, purification, and characterization of homogeneous ADC where p is a certain value from ADC with other drug loadings may be achieved by means such as reverse phase HPLC or electrophoresis.

For some antibody-drug conjugates, p may be limited by the number of attachment sites on the antibody. For example, where the attachment is a cysteine thiol, as in certain exemplary embodiments above, an antibody may have only one or several cysteine thiol groups, or may have only one or several sufficiently reactive thiol groups through which a linker may be attached. In certain embodiments, higher drug loading, e.g. p >5, may cause aggregation, insolubility, toxicity, or loss of cellular permeability of certain antibody-drug conjugates. In certain embodiments, the average drug loading for an ADC ranges from 1 to about 8; from about 2 to about 6; or from about 3 to about 5. Indeed, it has been shown that for certain ADCs, the optimal ratio of drug moieties per antibody may be less than 8, and may be about 2 to about 5 (U.S. Pat. No. 7,498,298).

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine.

The loading (drug/antibody ratio) of an ADC may be controlled in different ways, and for example, by: (i) limiting the molar excess of drug-linker intermediate or linker reagent relative to antibody, (ii) limiting the conjugation reaction time or temperature, and (iii) partial or limiting reductive conditions for cysteine thiol modification.

It is to be understood that where more than one nucleophilic group reacts with a drug-linker intermediate or linker reagent, then the resulting product is a mixture of ADC compounds with a distribution of one or more drug moieties attached to an antibody. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g. hydrophobic interaction chromatography (see, e.g., McDonagh et al (2006) Prot. Engr. Design & Selection 19(7):299-307; Hamblett et al (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value may be isolated from the conjugation mixture by electrophoresis or chromatography.

### d) Certain Methods of Preparing Immunoconjugates

An ADC of Formula I may be prepared by several routes employing organic chemistry reactions, conditions, and reagents known to those skilled in the art, including: (1) reaction of a nucleophilic group of an antibody with a bivalent linker reagent to form Ab-L via a covalent bond, followed by reaction with a drug moiety D; and (2) reaction of a nucleophilic group of a drug moiety with a bivalent linker reagent, to form D-L, via a covalent bond, followed by reaction with a nucleophilic group of an antibody. Exemplary methods for preparing an ADC of Formula I via the latter route are described in U.S. Pat. No. 7,498,298, which is expressly incorporated herein by reference.

Nucleophilic groups on antibodies include, but are not limited to: (i)N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into antibodies through modification of lysine residues, e.g., by reacting lysine residues with 2-iminothiolane (Traut's reagent), resulting in conversion of an amine into a thiol. Reactive thiol groups may also be introduced into an antibody by introducing one, two, three, four, or more cysteine residues (e.g., by preparing variant antibodies comprising one or more non-native cysteine amino acid residues).

Antibody-drug conjugates of the invention may also be produced by reaction between an electrophilic group on an antibody, such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In one embodiment, an antibody is modified to introduce electrophilic moieties that are capable of reacting with nucleophilic substituents on the linker reagent or drug. In another embodiment, the sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody that can react with appropriate groups on the drug (Hermanson, Bioconjugate Techniques). In another embodiment, antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a drug moiety or linker nucleophile.

Exemplary nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Nonlimiting exemplary cross-linker reagents that may be used to prepare ADC are described herein in the section titled “Exemplary Linkers.” Methods of using such cross-linker reagents to link two moieties, including a proteinaceous moiety and a chemical moiety, are known in the art. In some embodiments, a fusion protein comprising an antibody and a cytotoxic agent may be made, e.g., by recombinant techniques or peptide synthesis. A recombinant DNA molecule may comprise regions encoding the antibody and cytotoxic portions of the conjugate either adjacent to one another or separated by a region encoding a linker peptide which does not destroy the desired properties of the conjugate.

In yet another embodiment, an antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a drug or radionucleotide).

### E. Methods and Compositions for Diagnostics and Detection

In certain embodiments, any of the anti-CD22 antibodies provided herein is useful for detecting the presence of CD22 in a biological sample. The term “detecting” as used herein encompasses quantitative or qualitative detection. A “biological sample” comprises, e.g., a cell or tissue (e.g., biopsy material, including cancerous or potentially cancerous lymph tissue, including tissue from subjects having or suspected of having a B cell disorder and/or a B cell proliferative disorder, including, but not limited to, lymphoma, non-Hogkins lymphoma (NHL), aggressive NHL, relapsed aggressive NHL, relapsed indolent NHL, refractory NHL, refractory indolent NHL, chronic lymphocytic leukemia (CLL), small lymphocytic lymphoma, leukemia, hairy cell leukemia (HCL), acute lymphocytic leukemia (ALL), Burkitt's lymphoma, and mantle cell lymphoma.

In one embodiment, an anti-CD22 antibody for use in a method of diagnosis or detection is provided. In a further aspect, a method of detecting the presence of CD22 in a biological sample is provided. In certain embodiments, the method comprises contacting the biological sample with an anti-CD22 antibody as described herein under conditions permissive for binding of the anti-CD22 antibody to CD22, and detecting whether a complex is formed between the anti-CD22 antibody and CD22 in the biological sample. Such method may be an in vitro or in vivo method. In one embodiment, an anti-CD22 antibody is used to select subjects eligible for therapy with an anti-CD22 antibody, e.g. where CD22 is a biomarker for selection of patients. In a further embodiment, the biological sample is a cell or tissue (e.g., cancerous or potentially cancerous lymph tissue, including tissue of subjects having or suspected of having a B cell disorder and/or a B cell proliferative disorder, including, but not limited to, lymphoma, non-Hogkins lymphoma (NHL), aggressive NHL, relapsed aggressive NHL, relapsed indolent NHL, refractory NHL, refractory indolent NHL, chronic lymphocytic leukemia (CLL), small lymphocytic lymphoma, leukemia, hairy cell leukemia (HCL), acute lymphocytic leukemia (ALL), Burkitt's lymphoma, and mantle cell lymphoma.

In a further embodiment, an anti-CD22 antibody is used in vivo to detect, e.g., by in vivo imaging, a CD22-positive cancer in a subject, e.g., for the purposes of diagnosing, prognosing, or staging cancer, determining the appropriate course of therapy, or monitoring response of a cancer to therapy. One method known in the art for in vivo detection is immuno-positron emission tomography (immuno-PET), as described, e.g., in van Dongen et al., The Oncologist 12:1379-1389 (2007) and Verel et al., J. Nucl. Med. 44:1271-1281 (2003). In such embodiments, a method is provided for detecting a CD22-positive cancer in a subject, the method comprising administering a labeled anti-CD22 antibody to a subject having or suspected of having a CD22-positive cancer, and detecting the labeled anti-CD22 antibody in the subject, wherein detection of the labeled anti-CD22 antibody indicates a CD22-positive cancer in the subject. In certain of such embodiments, the labeled anti-CD22 antibody comprises an anti-CD22 antibody conjugated to a positron emitter, such as ⁶⁸Ga, ¹⁸F, ⁶⁴Cu, ⁸⁶Y, ⁷⁶Br, ⁸⁹Zr, and ¹²⁴I. In a particular embodiment, the positron emitter is ⁸⁹Zr.

In further embodiments, a method of diagnosis or detection comprises contacting a first anti-CD22 antibody immobilized to a substrate with a biological sample to be tested for the presence of CD22, exposing the substrate to a second anti-CD22 antibody, and detecting whether the second anti-CD22 is bound to a complex between the first anti-CD22 antibody and CD22 in the biological sample. A substrate may be any supportive medium, e.g., glass, metal, ceramic, polymeric beads, slides, chips, and other substrates. In certain embodiments, a biological sample comprises a cell or tissue (e.g., biopsy material, including cancerous or potentially cancerous lymph tissue, including tissue from subjects having or suspected of having a B cell disorder and/or a B cell proliferative disorder, including, but not limited to, lymphoma, non-Hogkins lymphoma (NHL), aggressive NHL, relapsed aggressive NHL, relapsed indolent NHL, refractory NHL, refractory indolent NHL, chronic lymphocytic leukemia (CLL), small lymphocytic lymphoma, leukemia, hairy cell leukemia (HCL), acute lymphocytic leukemia (ALL), Burkitt's lymphoma, and mantle cell lymphoma). In certain embodiments, the first or second anti-CD22 antibody is any of the antibodies described herein.

Exemplary disorders that may be diagnosed or detected according to any of the above embodiments include CD22-positive cancers, such as CD22-positive lymphoma, CD22-positive non-Hogkins lymphoma (NHL; including, but not limited to CD22-positive aggressive NHL, CD22-positive relapsed aggressive NHL, CD22-positive relapsed indolent NHL, CD22-positive refractory NHL, and CD22-positive refractory indolent NHL), CD22-positive chronic lymphocytic leukemia (CLL), CD22-positive small lymphocytic lymphoma, CD22-positive leukemia, CD22-positive hairy cell leukemia (HCL), CD22-positive acute lymphocytic leukemia (ALL), CD22-positive Burkitt's lymphoma, and CD22-positive mantle cell lymphoma. In some embodiments, a CD22-positive cancer is a cancer that receives an anti-CD22 immunohistochemistry (IHC) score greater than “0,” which corresponds to very weak or no staining in >90% of tumor cells. In some embodiments, a CD22-positive cancer expresses CD22 at a 1+, 2+ or 3+ level, wherein 1+ corresponds to weak staining in >50% of neoplastic cells, 2+ corresponds to moderate staining in >50% neoplastic cells, and 3+ corresponds to strong staining in >50% of neoplastic cells. In some embodiments, a CD22-positive cancer is a cancer that expresses CD22 according to an in situ hybridization (ISH) assay. In some such embodiments, a scoring system similar to that used for IHC is used. In some embodiments, a CD22-positive cancer is a cancer that expresses CD22 according to a reverse-transcriptase PCR (RT-PCR) assay that detects CD22 mRNA. In some embodiments, the RT-PCR is quantitative RT-PCR.

In certain embodiments, labeled anti-CD22 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like. In another embodiment, a label is a positron emitter. Positron emitters include but are not limited to ⁶⁸Ga, ¹⁸F, ⁶⁴Cu, ⁸⁶Y, ⁷⁶Br, ⁸⁹Zr, and ¹²⁴I. In a particular embodiment, a positron emitter is ⁸⁹Zr.

### F. Pharmaceutical Formulations

Pharmaceutical formulations of an anti-CD22 antibody or immunoconjugate as described herein are prepared by mixing such antibody or immunoconjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g. films, or microcapsules.

The formulations to be used for in vivo administration are generally sterile. Sterility may be readily accomplished, e.g., by filtration through sterile filtration membranes.

### G. Therapeutic Methods and Compositions

Any of the anti-CD22 antibodies or immunoconjugates provided herein may be used in methods, e.g., therapeutic methods.

In one aspect, an anti-CD22 antibody or immunoconjugate provided herein is used in a method of inhibiting proliferation of a CD22-positive cell, the method comprising exposing the cell to the anti-CD22 antibody or immunoconjugate under conditions permissive for binding of the anti-CD22 antibody or immunoconjugate to CD22 on the surface of the cell, thereby inhibiting the proliferation of the cell. In certain embodiments, the method is an in vitro or an in vivo method. In some embodiments, the cell is a B cell. In some embodiments, the cell is a neoplastic B cell, such as a lymphoma cell or a leukemia cell.

Inhibition of cell proliferation in vitro may be assayed using the CellTiter-Glo™ Luminescent Cell Viability Assay, which is commercially available from Promega (Madison, Wis.). That assay determines the number of viable cells in culture based on quantitation of ATP present, which is an indication of metabolically active cells. See Crouch et al. (1993) J. Immunol. Meth. 160:81-88, U.S. Pat. No. 6,602,677. The assay may be conducted in 96- or 384-well format, making it amenable to automated high-throughput screening (HTS). See Cree et al. (1995) AntiCancer Drugs 6:398-404. The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU).

In another aspect, an anti-CD22 antibody or immunoconjugate for use as a medicament is provided. In further aspects, an anti-CD22 antibody or immunoconjugate for use in a method of treatment is provided. In certain embodiments, an anti-CD22 antibody or immunoconjugate for use in treating CD22-positive cancer is provided. In certain embodiments, the invention provides an anti-CD22 antibody or immunoconjugate for use in a method of treating an individual having a CD22-positive cancer, the method comprising administering to the individual an effective amount of the anti-CD22 antibody or immunoconjugate. In one such embodiment, the method further comprises administering to the individual an effective amount of at least one additional therapeutic agent, e.g., as described below.

In a further aspect, the invention provides for the use of an anti-CD22 antibody or immunoconjugate in the manufacture or preparation of a medicament. In one embodiment, the medicament is for treatment of CD22-positive cancer. In a further embodiment, the medicament is for use in a method of treating CD22-positive cancer, the method comprising administering to an individual having CD22-positive cancer an effective amount of the medicament. In one such embodiment, the method further comprises administering to the individual an effective amount of at least one additional therapeutic agent, e.g., as described below.

In a further aspect, the invention provides a method for treating CD22-positive cancer. In one embodiment, the method comprises administering to an individual having such CD22-positive cancer an effective amount of an anti-CD22 antibody or immunoconjugate. In one such embodiment, the method further comprises administering to the individual an effective amount of at least one additional therapeutic agent, as described below.

A CD22-positive cancer according to any of the above embodiments may be, e.g., lymphoma, non-Hogkins lymphoma (NHL), aggressive NHL, relapsed aggressive NHL, relapsed indolent NHL, refractory NHL, refractory indolent NHL, chronic lymphocytic leukemia (CLL), small lymphocytic lymphoma, leukemia, hairy cell leukemia (HCL), acute lymphocytic leukemia (ALL), Burkitt's lymphoma, and mantle cell lymphoma. In some embodiments, a CD22-positive cancer is a cancer that receives an anti-CD22 immunohistochemistry (IHC) or in situ hybridization (ISH) score greater than “0,” which corresponds to very weak or no staining in >90% of tumor cells. In another embodiment, a CD22-positive cancer expresses CD22 at a 1+, 2+ or 3+ level, wherein 1+ corresponds to weak staining in >50% of neoplastic cells, 2+ corresponds to moderate staining in >50% neoplastic cells, and 3+ corresponds to strong staining in >50% of neoplastic cells. In some embodiments, a CD22-positive cancer is a cancer that expresses CD22 according to a reverse-transcriptase PCR (RT-PCR) assay that detects CD22 mRNA. In some embodiments, the RT-PCR is quantitative RT-PCR.

In some embodiments, immunoconjugates comprising a pyrrolobenzodiazepine cytotoxic moiety are useful for treating diffuse large B-cell lymphomas as evidenced, for example, by the xenograft models shown in Examples B and D. The immunoconjugate for use in treating diffuse large B-cell lymphomas may, in some embodiments, comprise a PBD dimer having the structure:

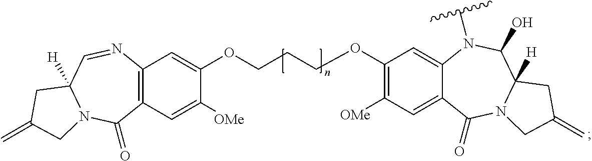

wherein n is 0 or 1. In some embodiments, the PBD dimer is covalently attached to the antibody through a protease cleavable linker, such as, for example, the immunoconjugate shown in FIG. 4A. In some embodiments, a PBD dimer is covalently attached to the antibody through a disulfide linker, such as, for example, the immunoconjugates shown in FIGS. 4B and 4C. In some embodiments, immunoconjugates comprising a pyrrolobenzodiazepine cytotoxic moiety are useful for treating mantle cell lymphoma and Burkitt's lymphoma.

An “individual” according to any of the above embodiments may be a human.

In a further aspect, the invention provides pharmaceutical formulations comprising any of the anti-CD22 antibodies or immunoconjugate provided herein, e.g., for use in any of the above therapeutic methods. In one embodiment, a pharmaceutical formulation comprises any of the anti-CD22 antibodies or immunoconjugates provided herein and a pharmaceutically acceptable carrier. In another embodiment, a pharmaceutical formulation comprises any of the anti-CD22 antibodies or immunoconjugates provided herein and at least one additional therapeutic agent, e.g., as described below.

Antibodies or immunoconjugates of the invention can be used either alone or in combination with other agents in a therapy. For instance, an antibody or immunoconjugate of the invention may be co-administered with at least one additional therapeutic agent.

In some embodiments, an anti-CD22 immunoconjugate is administered in combination with an anti-CD79b antibody or immunoconjugate. A nonlimiting exemplary anti-CD79b antibody or immunoconjugate comprises the hypervariable regions of huMA79bv28, such that the anti-CD79b antibody or immunoconjugate comprises (i) HVR H1 having the sequence of SEQ ID NO: 32, (ii) HVR H2 having the sequence of SEQ ID NO: 33, (iii) HVR H3 having the sequence of SEQ ID NO: 34, (iv) HVR L1 having the sequence of SEQ ID NO: 35, (v) HVR L2 having the sequence of SEQ ID NO: 36, and (vi) HVR L3 having the sequence of SEQ ID NO: 37. In some embodiments, an anti-CD79b antibody or immunoconjugate comprises the heavy chain variable region and light chain variable region of huMA79bv28. In some such embodiments, the anti-CD79b antibody or immunoconjugate comprises a heavy chain variable region having the sequence of SEQ ID NO: 38 and a light chain variable region having the sequence of SEQ ID NO: 39. An anti-CD79b immunoconjugate comprises, in some embodiments, a cytotoxic agent selected from an auristatin, a nemorubicin derivative, and a pyrrolobenzodiazepine. In some embodiments, an anti-CD79b immunoconjugate comprises a cytotoxic agent selected from MMAE, PNU-159682, and a PBD dimer having the structure:

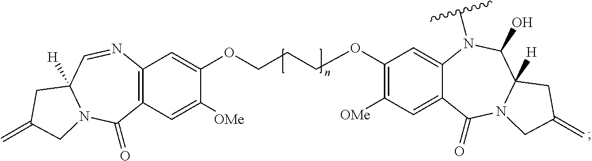

wherein n is 0 or 1. In some embodiments, an anti-CD79b immunoconjugate is selected from a thio huMA79bv28 HC A118C-MC-val-cit-PAB-MMAE immunoconjugate described, e.g., in U.S. Pat. No. 8,088,378 B2; a thio huMA79bv28 HC S400C-MC-val-cit-PAB-MMAE immunoconjugate, a thio huMA79bv28 LC V205C-MC-val-cit-PAB-MMAE immunoconjugate, a thio huMA79bv28 HC A118C-MC-val-cit-PAB-PNU-159682, a Thio huMA79bv28 HC A118C-MC-acetal-PNU-159682, a Thio huMA79bv28 HC A118C-MC-val-cit-PAB-PBD, a thio huMA79bv28 HC S400C-MC-val-cit-PAB-PNU-159682, a Thio huMA79bv28 HC S400C-MC-acetal-PNU-159682, a Thio huMA79bv28 HC S400C-MC-val-cit-PAB-PBD, a thio huMA79bv28 LC V205C-MC-val-cit-PAB-PNU-159682, a Thio huMA79bv28 LC V205C-MC-acetal-PNU-159682, and a Thio huMA79bv28 LC V205C-MC-val-cit-PAB-PBD. The heavy chain and light chain sequences for thio huMA79bv28 HC A118C are shown in SEQ ID NOs: 40 and 41, respectively. The heavy chain and light chain sequences for thio huMA79bv28 HC S400C are shown in SEQ ID NOs: 43 and 41, respectively. The heavy chain and light chain sequences for thio huMA79bv28 LC V205C are shown in SEQ ID NOs: 42 and 44, respectively. Apart from the specific antibody sequence, the structures of the anti-CD79b immunoconjugates are analogous to the structures of the anti-CD22 immunoconjugates described herein and in US 2008/0050310. Nonlimiting exemplary immunoconjugates comprising PNU-159682 have the structures:

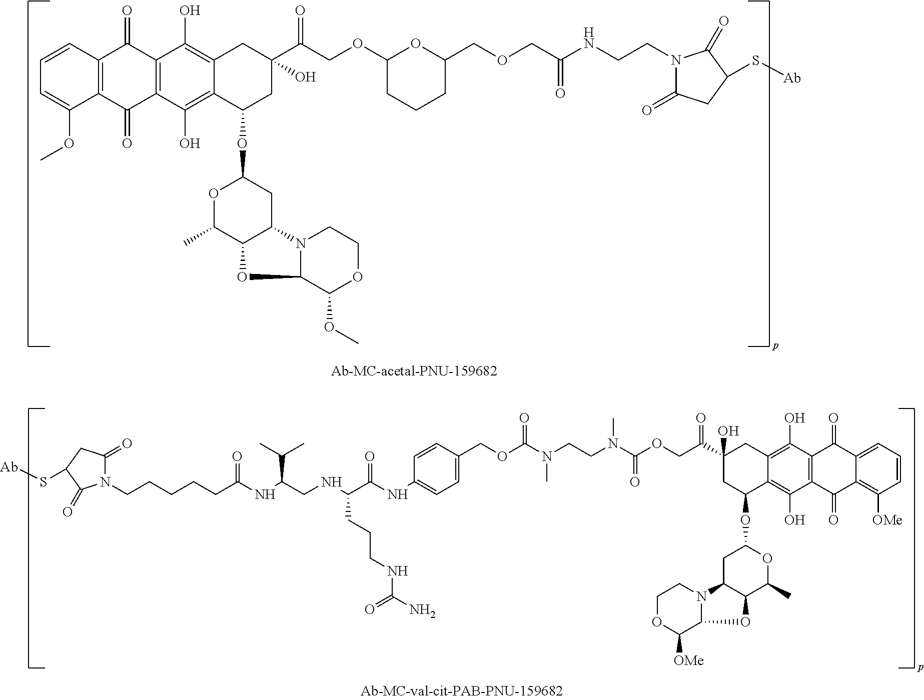

In some embodiments, an anti-CD22 immunoconjugate is administered in combination with an anti-CD20 antibody (either a naked antibody or an ADC). In some embodiments, the anti-CD20 antibody is rituximab (Rituxan®) or 2H7 (Genentech, Inc., South San Francisco, Calif.). In some embodiments, an anti-CD22 immunoconjugate is administered in combination with an anti-VEGF antibody (e.g, bevicizumab, trade name Avastin®).

Other therapeutic regimens may be combined with the administration of an anti-CD22 immunoconjugate, including, without limitation, radiation therapy and/or bone marrow and peripheral blood transplants, and/or a cytotoxic agent. In some embodiments, a cytotoxic agent is an agent or a combination of agents such as, for example, cyclophosphamide, hydroxydaunorubicin, adriamycin, doxorubincin, vincristine (Oncovin™), prednisolone, CHOP (combination of cyclophosphamide, doxorubicin, vincristine, and prednisolone), CVP (combination of cyclophosphamide, vincristine, and prednisolone), or immunotherapeutics such as anti-CD20 (e.g., rituximab, trade name Rituxan®), anti-VEGF (e.g., bevicizumab, trade name Avastin®), taxanes (such as paclitaxel and docetaxel) and anthracycline antibiotics.

Such combination therapies noted above encompass combined administration (where two or more therapeutic agents are included in the same or separate formulations), and separate administration, in which case, administration of the antibody or immunoconjugate of the invention can occur prior to, simultaneously, and/or following, administration of the additional therapeutic agent and/or adjuvant. Antibodies or immunoconjugates of the invention can also be used in combination with radiation therapy.

An antibody or immunoconjugate of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or immunoconjugates of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody or immunoconjugate need not be, but is optionally formulated with one or more agents currently used to prevent or treat the disorder in question. The effective amount of such other agents depends on the amount of antibody or immunoconjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or immunoconjugate of the invention (when used alone or in combination with one or more other additional therapeutic agents) will depend on the type of disease to be treated, the type of antibody or immunoconjugate, the severity and course of the disease, whether the antibody or immunoconjugate is administered for preventive or therapeutic purposes, previous therapy, the patient's clinical history and response to the antibody or immunoconjugate, and the discretion of the attending physician. The antibody or immunoconjugate is suitably administered to the patient at one time or over a series of treatments. Depending on the type and severity of the disease, about 1 μg/kg to 15 mg/kg (e.g. 0.1 mg/kg-10 mg/kg) of antibody or immunoconjugate can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody or immunoconjugate would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

In some embodiments, a lower dose of a 10F4v3 ADC comprising a pyrrolobenzodiazepine (PBD) dimer may be used to achieve the same efficacy as a higher dose of a 10F4v3 ADC comprising an MMAE moiety.

It is understood that any of the above formulations or therapeutic methods may be carried out using both an immunoconjugate of the invention and an anti-CD22 antibody.

### H. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disorder and may have a sterile access port (for example the container may be an intravenous solution bag or a vial having a stopper pierceable by a hypodermic injection needle). At least one active agent in the composition is an antibody or immunoconjugate of the invention. The label or package insert indicates that the composition is used for treating the condition of choice. Moreover, the article of manufacture may comprise (a) a first container with a composition contained therein, wherein the composition comprises an antibody or immunoconjugate of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

### III. Examples

The following are examples of methods and compositions of the invention. It is understood that various other embodiments may be practiced, given the general description provided above.

### A. Production of Anti-CD22 Antibody Drug Conjugates

Anti-CD22 antibody 10F4 and certain variants, including humanize variants hu10F4v1 and hu10F4v3, are described, e.g., in US 2008/0050310. Antibody 10F4, hu10F4v1, and hu10F4v3 comprise heavy chain HVRs of SEQ ID NO: 9, 10, and 11 (HVR H1, HVR H2, and HVR H3, respectively). Antibody 10F4 and hu10F4v1 comprise light chain HVRs of SEQ ID NO: 12, 13, and 14 (HVR L1, HVF L2, and HVR L3, respectively). Hu10F4v3 comprises light chain HVRs of SEQ ID NO: 15, 13, and 14 (HVR L1, HVF L2, and HVR L3, respectively), wherein the HVR L1 of hu10F4v3 comprises a single amino acid change (N₂₈V) relative to the HVR L1 of 10F4 and 10F4v1. The binding affinities of the three antibodies for human CD22 were found to be similar (ranging from 1.4 nM to 2.3 nM). Certain further amino acid substitutions were made in HVR L1 of hu10F4v1, and are shown in SEQ ID NOs: 16 to 22. Antibodies comprising each of those HVR L1 sequences had binding affinities for human CD22 that varied less than 2-fold from the binding affinity of hu10F4v1. See, e.g., US 2008/0050310.

For larger scale antibody production, antibodies were produced in CHO cells. Vectors coding for VL and VH were transfected into CHO cells and IgG was purified from cell culture media by protein A affinity chromatography.

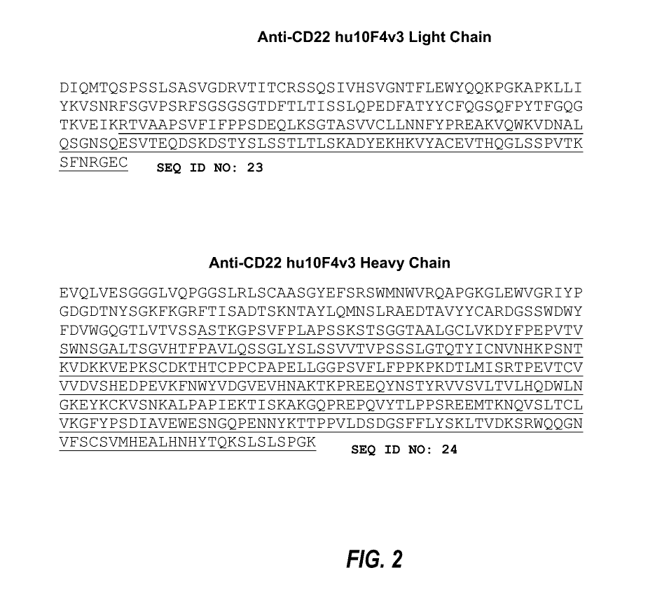

Anti-CD22 antibody-drug conjugates (ADCs) were produced by conjugating Thio Hu anti-CD22 10F4v3 HC A118C antibodies to certain drug moieties. Thio Hu anti-CD22 10F4v3 HC A118C is a humanized anti-CD22 10F4v3 antibody with an A118C mutation in the heavy chain that adds a conjugatable thiol group. See, e.g., US 2008/0050310. The amino acid sequence of the heavy chain of Thio Hu anti-CD22 10F4v3 HC A118C is shown in SEQ ID NO: 26 (see FIG. 3), and the amino acid sequence of the light chain of Thio Hu anti-CD22 10F4v3 HC A118C is shown in SEQ ID NO: 23 (see FIG. 2). The immunoconjugates were prepared as follows.

Thio Hu Anti-CD22 10F4v3 HC A118C-MC-Val-Cit-PAB-PBD (“10F4v3-PBD”)

Prior to conjugation, the antibody was reduced with dithiothreitol (DTT) to remove blocking groups (e.g. cysteine) from the engineered cysteines of the thio-antibody. This process also reduces the interchain disulfide bonds of the antibody. The reduced antibody was purified to remove the released blocking groups and the interchain disulfides were reoxidized using dehydro-ascorbic acid (dhAA). The intact antibody was then combined with the drug-linker moiety MC-val-cit-PAB-PBD (“val-cit” may also be referred to herein as “vc”) to allow conjugation of the drug-linker moiety to the engineered cysteine residues of the antibody. The conjugation reaction was quenched by adding excess N-acetyl-cysteine to react with any free linker-drug moiety, and the ADC was purified. The drug load (average number of drug moieties per antibody) for the ADC was in the range of about 1.7 to about 1.9, as indicated in the Examples below. 10F4v3-PBD has the structure shown in FIG. 4A (p=drug load).

Thio Hu Anti-CD22 10F4v3 HC A118C-MC-Val-Cit-PAB-MMAE (“10F4v3-MMAE”)

Prior to conjugation, the antibody was reduced with dithiothreitol (DTT) to remove blocking groups (e.g. cysteine) from the engineered cysteines of the thio-antibody. This process also reduces the interchain disulfide bonds of the antibody. The reduced antibody was purified to remove the released blocking groups and the interchain disulfides were reoxidized using dehydro-ascorbic acid (dhAA). The intact antibody was then combined with the drug-linker moiety MC-val-cit-PAB-MMAE (“val-cit” may also be referred to herein as “vc”) to allow conjugation of the drug-linker moiety to the engineered cysteine residues of the antibody. The conjugation reaction was quenched by adding excess N-acetyl-cysteine to react with any free linker-drug moiety, and the ADC was purified. The drug load (average number of drug moieties per antibody) for the ADC was determined to be about 2, as indicated in the examples below. Thio Hu anti-CD22 10F4v3 HC A118C-MC-val-cit-PAB-MMAE is described, e.g., in US 2008/0050310.

Thio Hu Anti-CD22 10F4v3 HC A118C-Disulfide-PBD (“10F4v3-SS-PBD”) and Thio Hu Anti-CD22 10F4v3 HC A118C-Disulfide Methyl-PBD (“10F4v3-SSMe-PBD”)

Prior to conjugation, the antibody was reduced with dithiothreitol (DTT) to remove blocking groups (e.g. cysteine) from the engineered cysteines of the thio-antibody. This process also reduces the interchain disulfide bonds of the antibody. The reduced antibody was purified to remove the released blocking groups and the interchain disulfides were reoxidized using dehydro-ascorbic acid (dhAA).

The intact antibody was then combined with a 6-8 fold molar excess of the drug-linker moiety (14 or 22 from Example H or I, respectively) in 50 mM Tris, pH 8, for 16 to 24 hours. The ADC was then purified by cation exchange column. The drug load (average number of drug moieties per antibody) for the ADC was in the range of about 1.7 to about 1.9, as indicated in the Examples below. 10F4v3-SS-PBD has the structure shown in FIG. 4B (p=drug load). 10F4v3-SSMe-PBD has the structure shown in FIG. 4C (p=drug load).

### B. In Vivo Anti-Tumor Activity of Humanized Anti-CD22 Antibody Drug Conjugates in a WSU-DLCL2 Xenograft Model

To test the efficacy of Thio Hu anti-CD22 10F4v3 HC A118C conjugate with PBD, the effects of the conjugated antibodies in a mouse xenograft model of WSU-DLCL2 tumors (diffuse large B-cell lymphoma cell line) was examined.

Female CB17 ICR SCID mice (12-13 weeks of age from Charles Rivers Laboratories; Hollister, Calif.) were each inoculated subcutaneously in the flank with 2×10⁷ WSU-DLCL2 cells (DSMZ, German Collection of Microorganisms and Cell Cultures, Braunschweig, Germany). When the xenograft tumors reached an average tumor volume of 150-300 mm³ (referred to as Day 0), the first and only dose of treatment was administered. Tumor volume was calculated based on two dimensions, measured using calipers, and was expressed in mm³ according to the formula: V=0.5a×b², wherein a and b are the long and the short diameters of the tumor, respectively. To analyze the repeated measurement of tumor volumes from the same animals over time, a mixed modeling approach was used (see, e.g., Pinheiro J, et al. nlme: linear and nonlinear mixed effects models. 2009; R package, version 3.1-96). This approach can address both repeated measurements and modest dropout rates due to non-treatment related removal of animals before the study end. Cubic regression splines were used to fit a non-linear profile to the time courses of log 2 tumor volume at each dose level. These non-linear profiles were then related to dose within the mixed model.

Groups of 9 mice were treated with a single intravenous (i.v.) dose of 0.5 or 2 or 8 mg ADC/kg of Thio Hu anti-CD22 10F4v3 HC A118C immunoconjugate or control antibody-drug conjugates (control ADCs). The control ADCs bind to a protein that is not expressed on the surface of WSU-DLCL2 cells. Tumors and body weights of mice were measured 1-2 times a week throughout the experiment. Mice were euthanized before tumor volumes reached 3000 mm³ or when tumors showed signs of impending ulceration. All animal protocols were approved by an Institutional Animal Care and Use Committee (IACUC).

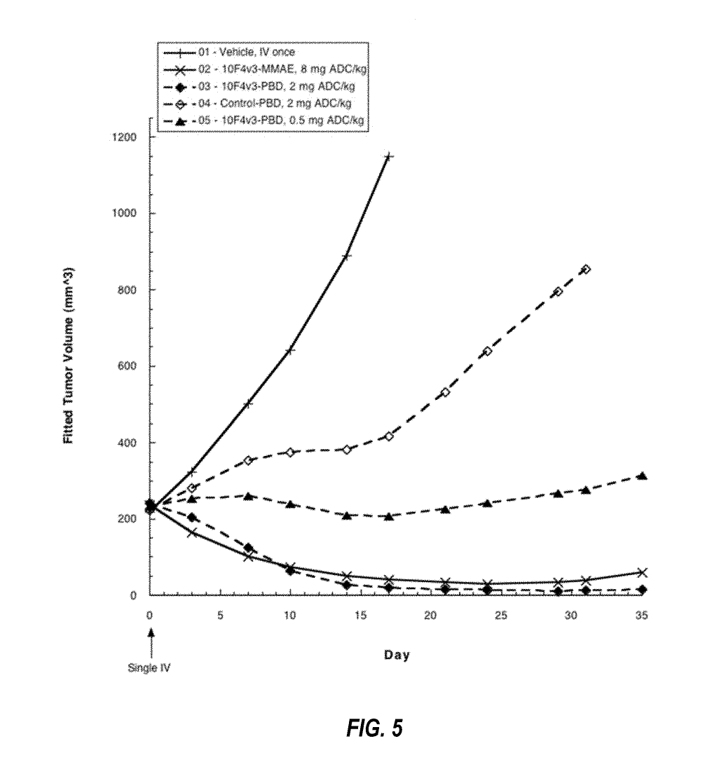

The results of that experiment are shown in Table 2 and FIG. 5. Table 2 shows each treatment group, the number of mice with observable tumors at the end of the study (“TI”), the number of mice showing a partial response (“PR”; where the tumor volume at any time after administration dropped below 50% of the tumor volume measured at day 0), the number of mice showing a complete response (“CR”; where the tumor volume at any time after administration dropped to 0 mm³), the drug dose for each group, the antibody dose for each group, and the drug load for each ADC administered.

TABLE 2
Anti-CD22 ADC administration to mice with WSU-DLCL2 xenografts

| | | | | | | Drug |
|---|---|---|---|---|---|---|
| | | | | Drug | Ab | Load |
| Antibody administered | | | | Dose | Dose | (Drug/ |
| (Treatment) | TI | PR | CR | (μg//kg) | (mg/kg) | Ab) |
| Vehicle* | 9/9 | 0 | 0 | n/a | n/a | n/a |
| 10F4v3-PBD | 9/9 | 2 | 0 | 3.41 | 0.5 | 1.75 |
| 10F4v3-PBD | 3/9 | 0 | 9 | 13.64 | 2 | 1.75 |
| Control ADC-MC-val- | 9/9 | 0 | 0 | 14.03 | 2 | 1.8 |
| cit-PAB-PBD (“Control- | | | | | | |
| PBD”) | | | | | | |
| Thio Hu anti-CD22 10F4v3 | 7/9 | 6 | 3 | 76.59 | 8 | 2 |
| HC A118C-MC-vc- | | | | | | |
| PAB-MMAE | | | | | | |
| (“10F4v3-MMAE”) | | | | | | |

*Vehicle = 20 mM histidine acetate, pH 5.5, 240 mM sucrose, 0.02% PS20;
n/a = not applicable.

In a 35 day time course with drug conjugates and doses as shown in Table 2, 10F4v3 ADCs conjugated through a protease cleavable linker with PBD (“10F4v3-PBD”) showed inhibition of tumor growth in SCID mice with WSU-DLCL2 tumors compared to the vehicle and the control ADC (“Control-PBD”). See FIG. 5.

Furthermore, 2 mg/kg of the 10F4v3-PBD showed comparable anti-tumor activity as 8 mg/kg of the humanized anti-CD22 thiomab conjugated with auristatin drug MMAE (“10F4v3-MMAE”). See FIG. 5. As shown in Table 2, mice receiving 2 mg/kg 10F4v3-PBD had nine complete responses, whereas mice receiving 8 mg/kg 10F4v3-MMAE had six partial responses and three complete responses.

In this study, the percent body weight change was determined in each dosage group. The results indicated that administration of the 10F4v3 ADCs did not cause a significant decrease in body weight during the study.

### C. In Vivo Anti-Tumor Activity of Humanized Anti-CD22 Antibody Drug Conjugates in a Granta-519 Xenograft Model

To test the efficacy of Thio Hu anti-CD22 10F4v3 HC A118C conjugate with PBD (“10F4v3-PBD”), the effects of the conjugated antibodies in a mouse xenograft model of Granta-519 tumors (human mantle cell lymphoma cell line) was examined.

Female CB17 ICR SCID mice (10-11 weeks of age from Charles Rivers Laboratories; Hollister, Calif.) were each inoculated subcutaneously in the flank with 2×10⁷ Granta-519 cells (DSMZ, German Collection of Microorganisms and Cell Cultures, Braunschweig, Germany). When the xenograft tumors reached an average tumor volume of 150-300 mm³ (referred to as Day 0), the first and only dose of treatment was administered. Tumor volume was calculated based on two dimensions, measured using calipers, and was expressed in mm³ according to the formula: V=0.5a×b², wherein a and b are the long and the short diameters of the tumor, respectively. To analyze the repeated measurement of tumor volumes from the same animals over time, a mixed modeling approach was used (see, e.g., Pinheiro et al. 2009). This approach can address both repeated measurements and modest dropout rates due to non-treatment related removal of animals before the study end. Cubic regression splines were used to fit a non-linear profile to the time courses of log 2 tumor volume at each dose level. These non-linear profiles were then related to dose within the mixed model.

Groups of 9 mice were treated with a single intravenous (i.v.) dose of 1 mg ADC/kg of 10F4v3 immunoconjugate or control antibody-drug conjugates (control ADCs). The control ADCs bind to a protein that is not expressed on the surface of Grant-519 cells. Tumors and body weights of mice were measured 1-2 times a week throughout the experiment. Mice were euthanized before tumor volumes reached 3000 mm³ or when tumors showed signs of impending ulceration. All animal protocols were approved by an Institutional Animal Care and Use Committee (IACUC).

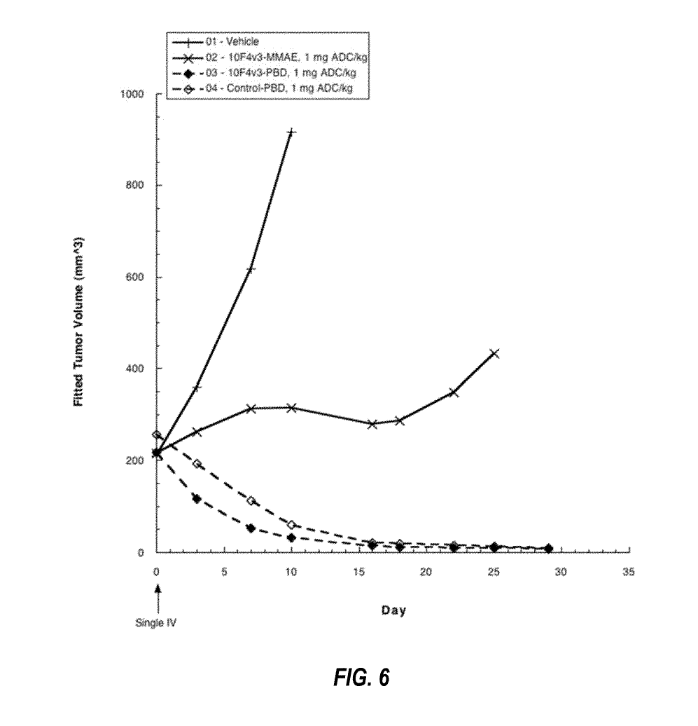

The results of that experiment are shown in Table 3 and FIG. 6. Table 3 shows each treatment group, the number of mice with observable tumors at the end of the study (“TI”), the number of mice showing a partial response (“PR”; where the tumor volume at any time after administration dropped below 50% of the tumor volume measured at day 0), the number of mice showing a complete response (“CR”; where the tumor volume at any time after administration dropped to 0 mm³), the drug dose for each group, the antibody dose for each group, and the drug load for each ADC administered.

TABLE 3
Anti-CD22 ADC administration to mice with Grant-519 xenografts

| | | | | | | Drug |
|---|---|---|---|---|---|---|
| | | | | Drug | | Load |
| Antibody administered | | | | Dose - | Ab Dose | (Drug/ |
| (Treatment) | TI | PR | CR | (μg//kg) | (mg/kg) | Ab) |
| Vehicle* | 9/9 | 0 | 0 | n/a | n/a | n/a |
| 10F4v3-PBD | 1/9 | 1 | 8 | 6.82 | 1 | 1.75 |
| Control-PBD | 2/9 | 2 | 7 | 7.02 | 1 | 1.8 |
| 10F4v3-MMAE | 8/9 | 0 | 1 | 9.57 | 1 | 2 |

*Vehicle = 20 mM histidine acetate, pH 5.5, 240 mM sucrose, 0.02% PS20;
n/a = not applicable.

In a 29 day time course with 1 mg ADC/kg doses of the drug conjugates as shown in Table 3, Thio Hu anti-CD22 ADC conjugated through a protease cleavable linker with PBD (“10F4v3-PBD”) showed inhibition of tumor growth in SCID mice with Granta-519 tumors compared to the vehicle. However, the control ADC conjugated to PBD (“Control-PBD”) also showed anti-tumor activity, indicating that this tumor model is very sensitive to PBD. Finally, when given at 1 mg/kg, 10F4v3-PBD better inhibited tumor growth than the humanized anti-CD22 thiomab conjugated with auristatin drug MMAE (“10F4v3-MMAE”).

Mice receiving 10F4v3-PBD all showed tumor regression, while majority of mice treated with 10F4v3-MMAE did not. A single dose of 10F4v3-PBD resulted in one partial response and eight complete responses.

In this study, the percent body weight change was determined in each dosage group. The results indicated that administration of the 10F4v3 ADCs did not cause a significant decrease in body weight during the study.

### D. In Vivo Anti-Tumor Activity of Humanized Anti-CD22 Antibody Drug Conjugates in a SuDHL4-Luc Xenograft Model

To test the efficacy of Thio Hu anti-CD22 10F4v3 HC A118C conjugate with PBD (“10F4v3-PBD”), the effects of the conjugated antibodies in a mouse xenograft model of SuDHL4-luc tumors (diffuse large B-cell lymphoma cell line) was examined.

Female CB17 ICR SCID mice (11-12 weeks of age from Charles Rivers Laboratories; Hollister, Calif.) were each inoculated subcutaneously in the flank with 2×10⁷ SuDHL4-luc cells (obtained from DSMZ, German Collection of Microorganisms and Cell Cultures, Braunschweig, Germany, and engineered at Genentech to stably express a luciferase gene). When the xenograft tumors reached an average tumor volume of 150-300 mm³ (referred to as Day 0), the first and only dose of treatment was administered. Tumor volume was calculated based on two dimensions, measured using calipers, and was expressed in mm³ according to the formula: V=0.5a×b², wherein a and b are the long and the short diameters of the tumor, respectively. To analyze the repeated measurement of tumor volumes from the same animals over time, a mixed modeling approach was used (see, e.g., Pinheiro et al. 2008). This approach can address both repeated measurements and modest dropout rates due to non-treatment related removal of animals before the study end. Cubic regression splines were used to fit a non-linear profile to the time courses of log 2 tumor volume at each dose level. These non-linear profiles were then related to dose within the mixed model.

Groups of 8 mice were treated with a single intravenous (i.v.) dose of 2 or 8 mg ADC/kg of 10F4v3 immunoconjugate or control antibody-drug conjugates (control ADCs). The control ADCs bind to a protein that is not expressed on the surface of SuDHL4-luc cells. Tumors and body weights of mice were measured 1-2 times a week throughout the experiment. Mice were euthanized before tumor volumes reached 3000 mm³ or when tumors showed signs of impending ulceration. All animal protocols were approved by an Institutional Animal Care and Use Committee (IACUC).

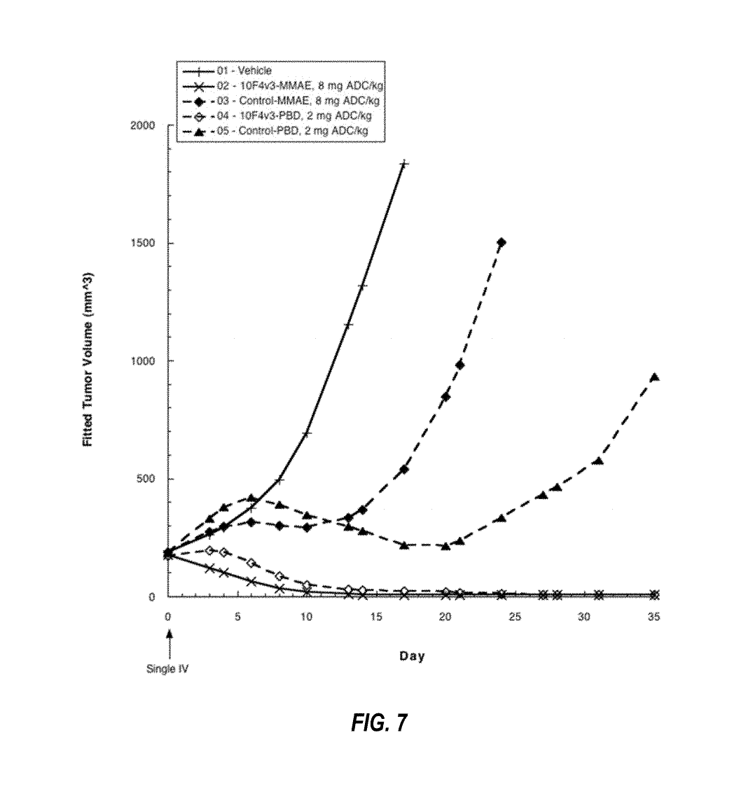

The results of that experiment are shown in Table 4 and FIG. 7. Table 4 shows each treatment group, the number of mice with observable tumors at the end of the study (“TI”), the number of mice showing a partial response (“PR”; where the tumor volume at any time after administration dropped below 50% of the tumor volume measured at day 0), the number of mice showing a complete response (“CR”; where the tumor volume at any time after administration dropped to 0 mm³), the drug dose for each group, the antibody dose for each group, and the drug load for each ADC administered.

TABLE 4
Anti-CD22 ADC administration to mice with SuDHL4-luc xenografts

| | | | | | | Drug |
|---|---|---|---|---|---|---|
| | | | | Drug | Ab | Load |
| Antibody administered | | | | Dose | Dose | (Drug/ |
| (Treatment) | TI | PR | CR | (μg//kg) | (mg/kg) | Ab) |
| Vehicle* | 8/8 | 0 | 0 | n/a | n/a | n/a |
| 10F4v3-PBD | 0/8 | 0 | 8 | 13.64 | 2 | 1.75 |
| Control-PBD | 8/8 | 2 | 0 | 14.03 | 2 | 1.8 |
| 10F4v3-MMAE | 0/8 | 0 | 8 | 76.59 | 8 | 2 |
| Control-ADC-A118C- | 8/8 | 0 | 0 | 80.42 | 8 | 2.1 |
| MC-vc-PAB-MMAE | | | | | | |
| (“Control MMAE”) | | | | | | |

*Vehicle = 20 mM histidine acetate, pH 5.5, 240 mM sucrose, 0.02% PS20;
n/a = not applicable.

In a 35 day time course with drug conjugates and doses as shown in Table 4, Thio Hu anti-CD22 ADC conjugated through a protease cleavable linker with PBD (“10F4v3-PBD”) showed inhibition of tumor growth in SCID mice with SuDHL4-luc tumors compared to the vehicle and the control ADC (“Control-PBD”). See FIG. 7.

Furthermore, 2 mg/kg of the 10F4v3-PBD showed comparable anti-tumor activity as 8 mg/kg of the humanized anti-CD22 thiomab conjugated with auristatin drug MMAE (“10F4v3-MMAE”); both showed a complete response in all treated animals. See FIG. 7 and Table 4.

In this study, the percent body weight change was determined in each dosage group. The results indicated that administration of the 10F4v3 ADCs did not cause a significant decrease in body weight during the study.

### E. Dose Escalation Study of 10F4v3-PBD in a SuDHL4-Luc Xenograft Model

The efficacy of 10F4v3-PBD at various dose levels in a mouse xenograft model of SuDHL4-luc tumors (diffuse large B-cell lymphoma cell line) was examined.

Female CB17 ICR SCID mice (9-10 weeks of age from Charles Rivers Laboratories; Hollister, Calif.) were each inoculated subcutaneously in the flank with 2×10⁷ SuDHL4-luc cells (obtained from DSMZ, German Collection of Microorganisms and Cell Cultures, Braunschweig, Germany, and engineered at Genentech to stably express a luciferase gene). When the xenograft tumors reached an average tumor volume of 150-300 mm³ (referred to as Day 0), the first and only dose of treatment was administered. Tumor volume was calculated based on two dimensions, measured using calipers, and was expressed in mm³ according to the formula: V=0.5a×b², wherein a and b are the long and the short diameters of the tumor, respectively. To analyze the repeated measurement of tumor volumes from the same animals over time, a mixed modeling approach was used (see, e.g., Pinheiro et al. 2008). This approach can address both repeated measurements and modest dropout rates due to non-treatment related removal of animals before the study end. Cubic regression splines were used to fit a non-linear profile to the time courses of log 2 tumor volume at each dose level. These non-linear profiles were then related to dose within the mixed model.

Groups of 8 mice were treated with a single intravenous (i.v.) dose of 0.2, 0.5, 1, or 2 mg ADC/kg of 10F4v3-PBD or Control-PBD, which binds to a protein that is not expressed on the surface of SuDHL4-luc cells. Tumors and body weights of mice were measured 1-2 times a week throughout the experiment. Mice were euthanized before tumor volumes reached 3000 mm³ or when tumors showed signs of impending ulceration. All animal protocols were approved by an Institutional Animal Care and Use Committee (IACUC).

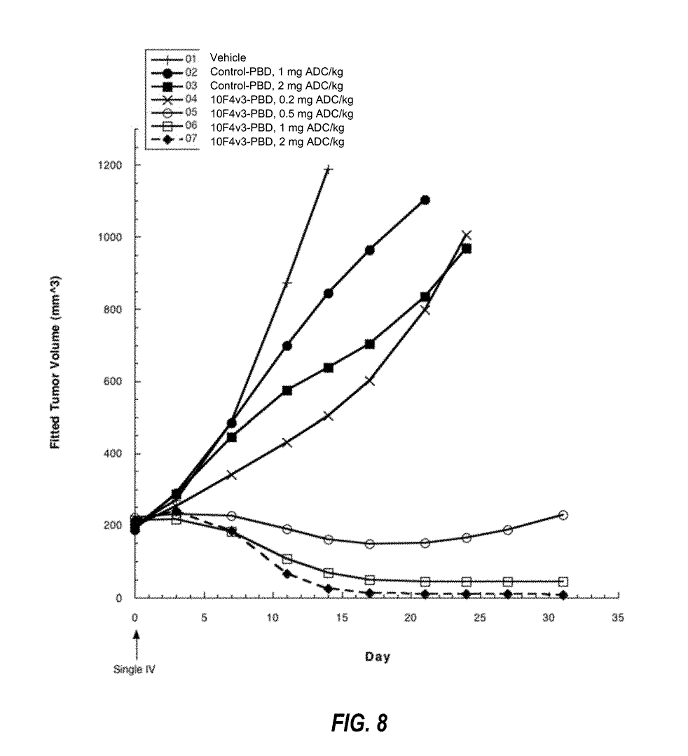

The results of that experiment are shown in Table 5 and FIG. 8. Table 5 shows each treatment group, the number of mice with observable tumors at the end of the study (“TI”), the number of mice showing a partial response (“PR”; where the tumor volume at any time after administration dropped below 50% of the tumor volume measured at day 0), the number of mice showing a complete response (“CR”; where the tumor volume at any time after administration dropped to 0 mm³), the drug dose for each group, the antibody dose for each group, and the drug load for each ADC administered.

TABLE 5
Anti-CD22 ADC administration to mice with SuDHL4-luc xenografts

| | | | | | | Drug |
|---|---|---|---|---|---|---|
| | | | | Drug | Ab | Load |
| Antibody administered | | | | Dose | Dose | (Drug/ |
| (Treatment) | TI | PR | CR | (μg//kg) | (mg/kg) | Ab) |
| Vehicle* | 8/8 | 0 | 0 | n/a | n/a | n/a |
| 10F4v3-PBD | 8/8 | 0 | 0 | 1.33 | 0.2 | 1.7 |
| 10F4v3-PBD | 7/8 | 2 | 1 | 3.31 | 0.5 | 1.7 |
| 10F4v3-PBD | 5/8 | 1 | 4 | 6.63 | 1 | 1.7 |
| 10F4v3-PBD | 0/8 | 0 | 8 | 13.25 | 2 | 1.7 |
| Control-PBD | 8/8 | 0 | 0 | 7.02 | 1 | 1.8 |
| Control-PBD | 8/8 | 0 | 0 | 14.03 | 2 | 1.8 |

*Vehicle = 20 mM histidine acetate, pH 5.5, 240 mM sucrose, 0.02% PS20;
n/a = not applicable.

In a 31 day time course with drug conjugates and doses as shown in Table 5, 10F4v3-PBD showed dose-dependent inhibition of tumor growth in SCID mice with SuDHL4-luc tumors. When administered at 0.5 mg/kg or higher dose, 10F4v3-PBD showed clear inhibitory activity compared to vehicle or the control ADC. See FIG. 8. In addition, a single dose of 2 mg/kg 10F4v3-PBD resulted in complete tumor remission in all treated animals.

In this study, the percent body weight change was determined in each dosage group. The results indicated that administration of 10F4v3-PBD did not cause a significant decrease in body weight during the study.

### F. Dose Escalation Study of 10F4v3-PBD in a Bjab-Luc Xenograft Model

The efficacy of 10F4v3-PBD at various dose levels in a mouse xenograft model of Bjab-luc tumors (Burkitt's lymphoma cell line) was examined.

Female CB17 ICR SCID mice (11-12 weeks of age from Charles Rivers Laboratories; Hollister, Calif.) were each inoculated subcutaneously in the flank with 2×10⁷ Bjab-luc cells (available, e.g., from Lonza, Basel, Switzerland, and engineered at Genentech to stably express a luciferase gene). When the xenograft tumors reached an average tumor volume of 150-300 mm³ (referred to as Day 0), the first and only dose of treatment was administered. Tumor volume was calculated based on two dimensions, measured using calipers, and was expressed in mm³ according to the formula: V=0.5a×b², wherein a and b are the long and the short diameters of the tumor, respectively. To analyze the repeated measurement of tumor volumes from the same animals over time, a mixed modeling approach was used (see, e.g., Pinheiro et al. 2008). This approach can address both repeated measurements and modest dropout rates due to non-treatment related removal of animals before the study end. Cubic regression splines were used to fit a non-linear profile to the time courses of log 2 tumor volume at each dose level. These non-linear profiles were then related to dose within the mixed model.

Groups of 9 mice were treated with a single intravenous (i.v.) dose of 0.05, 0.2, 0.5, or 1 mg ADC/kg of 10F4v3-PBD or Control-PBD, which binds to a protein that is not expressed on the surface of Bjab-luc cells. Tumors and body weights of mice were measured 1-2 times a week throughout the experiment. Mice were euthanized before tumor volumes reached 3000 mm³ or when tumors showed signs of impending ulceration. All animal protocols were approved by an Institutional Animal Care and Use Committee (IACUC).

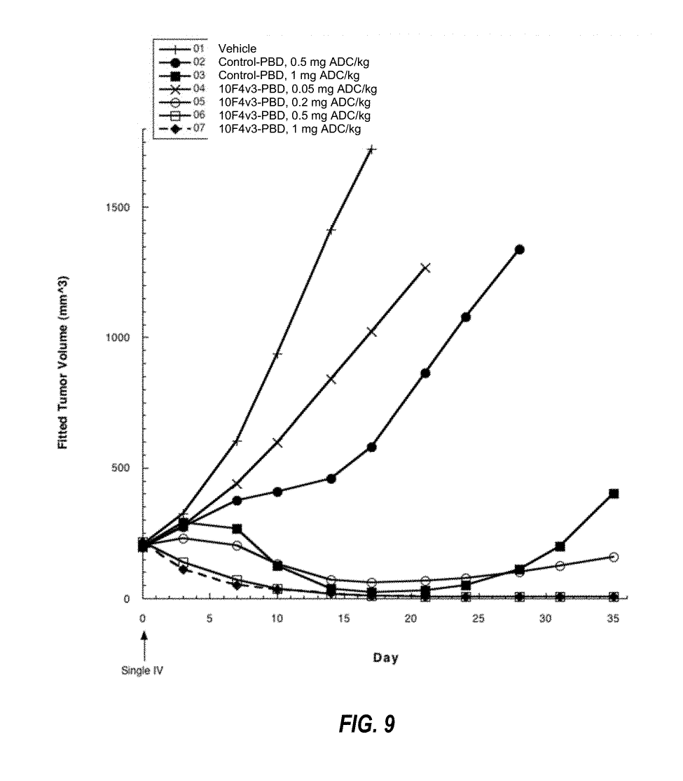

The results of that experiment are shown in Table 6 and FIG. 9. Table 6 shows each treatment group, the number of mice with observable tumors at the end of the study (“TI”), the number of mice showing a partial response (“PR”; where the tumor volume at any time after administration dropped below 50% of the tumor volume measured at day 0), the number of mice showing a complete response (“CR”; where the tumor volume at any time after administration dropped to 0 mm³), the drug dose for each group, the antibody dose for each group, and the drug load for each ADC administered.

TABLE 6
Anti-CD22 ADC administration to mice with Bjab-luc xenografts

| | | | | | | Drug |
|---|---|---|---|---|---|---|
| | | | | Drug | Ab | Load |
| Antibody administered | | | | Dose | Dose | (Drug/ |
| (Treatment) | TI | PR | CR | (μg//kg) | (mg/kg) | Ab) |
| Vehicle* | 9/9 | 0 | 0 | n/a | n/a | n/a |
| 10F4v3-PBD | 9/9 | 0 | 0 | 0.36 | 0.05 | 1.87 |
| 10F4v3-PBD | 7/9 | 5 | 2 | 1.46 | 0.2 | 1.87 |
| 10F4v3-PBD | 0/9 | 0 | 9 | 3.64 | 0.5 | 1.87 |
| 10F4v3-PBD | 0/9 | 0 | 9 | 7.29 | 1 | 1.87 |
| Control-PBD | 9/9 | 0 | 0 | 3.51 | 0.5 | 1.8 |
| Control-PBD | 9/9 | 4 | 4 | 7.02 | 1 | 1.8 |

*Vehicle = 20 mM histidine acetate, pH 5.5, 240 mM sucrose, 0.02% PS20;
n/a = not applicable.

In a 35 day time course with drug conjugates and doses as shown in Table 6, 10F4v3-PBD showed dose-dependent inhibition of tumor growth in SCID mice with Bjab-luc tumors. When administered at 0.2 mg/kg or higher dose, 10F4v3-PBD showed clear inhibitory activity compared to vehicle or the control ADC. See FIG. 9. In addition, a single dose of 0.5 or 1 mg/kg 10F4v3-PBD resulted in complete tumor remission in all treated animals. Control-PBD at 1 mg/kg also showed substantial anti-tumor activity, indicating that this model is very sensitive to PBD.

In this study, the percent body weight change was determined in each dosage group. The results indicated that administration of 10F4v3-PBD did not cause a significant decrease in body weight during the study.

### G. In Vivo Anti-Tumor Activity of Humanized Anti-CD22 Antibody Drug Conjugates in a WSU-DLCL2 Xenograft Model

To test the efficacy of Thio Hu anti-CD22 10F4v3 HC A118C conjugates with PBD, the effects of the conjugated antibodies in a mouse xenograft model of WSU-DLCL2 tumors (diffuse large B-cell lymphoma cell line) was examined.

Female CB17 ICR SCID mice (9-10 weeks of age from Charles Rivers Laboratories; Hollister, Calif.) were each inoculated subcutaneously in the flank with 2×10⁷ WSU-DLCL2 cells (DSMZ, German Collection of Microorganisms and Cell Cultures, Braunschweig, Germany). When the xenograft tumors reached an average tumor volume of 150-300 mm³ (referred to as Day 0), the first and only dose of treatment was administered. Tumor volume was calculated based on two dimensions, measured using calipers, and was expressed in mm³ according to the formula: V=0.5a×b², wherein a and b are the long and the short diameters of the tumor, respectively. To analyze the repeated measurement of tumor volumes from the same animals over time, a mixed modeling approach was used (see, e.g., Pinheiro J, et al. nlme: linear and nonlinear mixed effects models. 2009; R package, version 3.1-96). This approach can address both repeated measurements and modest dropout rates due to non-treatment related removal of animals before the study end. Cubic regression splines were used to fit a non-linear profile to the time courses of log 2 tumor volume at each dose level. These non-linear profiles were then related to dose within the mixed model.

Groups of 9 mice were treated with a single intravenous (i.v.) dose of 0.5 or 2 or 10 mg ADC/kg of Thio Hu anti-CD22 10F4v3 HC A118C immunoconjugate or control antibody-drug conjugates (control ADCs). The control ADCs bind to a protein that is not expressed on the surface of WSU-DLCL2 cells. Tumors and body weights of mice were measured 1-2 times a week throughout the experiment. Mice were euthanized before tumor volumes reached 3000 mm³ or when tumors showed signs of impending ulceration. All animal protocols were approved by an Institutional Animal Care and Use Committee (IACUC).

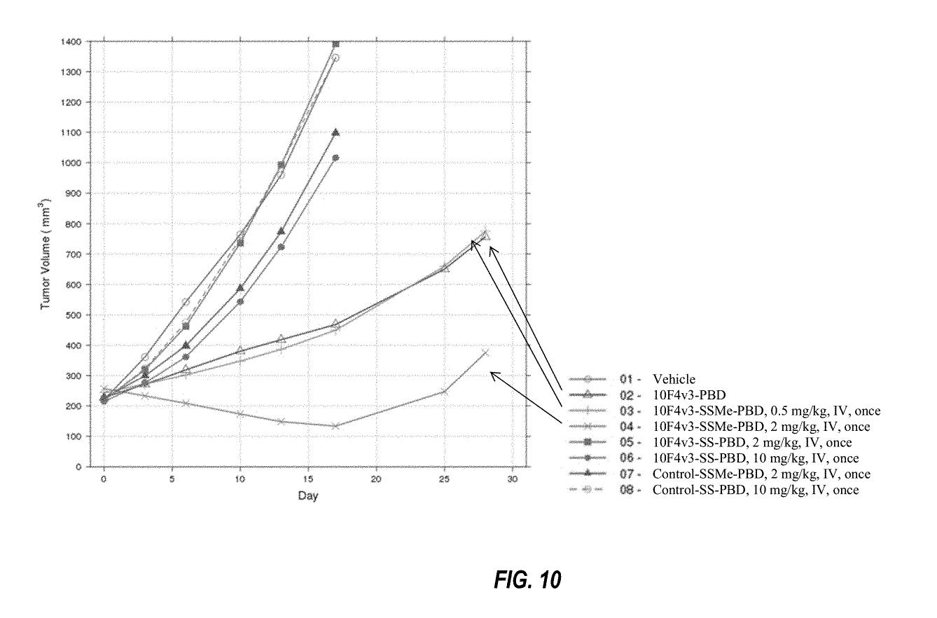

The results of that experiment are shown in Table 7 and FIG. 10. Table 2 shows each treatment group, the number of mice with observable tumors at the end of the study (“TI”), the number of mice showing a partial response (“PR”; where the tumor volume at any time after administration dropped below 50% of the tumor volume measured at day 0), the number of mice showing a complete response (“CR”; where the tumor volume at any time after administration dropped to 0 mm³), the drug dose for each group, the antibody dose for each group, and the drug load for each ADC administered.

TABLE 7
Anti-CD22 ADC administration to mice with WSU-DLCL2 xenografts

| | | | | | | Drug |
|---|---|---|---|---|---|---|
| | | | | Drug | Ab | Load |
| Antibody administered | | | | Dose | Dose | (Drug/ |
| (Treatment) | TI | PR | CR | (μg//kg) | (mg/kg) | Ab) |
| Vehicle* | 9 | 0 | 0 | n/a | n/a | n/a |
| 10F4v3-PBD | 9 | 0 | 0 | 3.31 | 0.5 | 1.7 |
| 10F4v3-SSMe-PBD | 9 | 1 | 0 | 3.51 | 0.5 | 1.8 |
| 10F4v3-SSMe-PBD | 9 | 2 | 0 | 14.03 | 2 | 1.8 |
| 10F4v3-SS-PBD | 9 | 0 | 0 | 13.25 | 2 | 1.7 |
| 10F4v3-SS-PBD | 9 | 0 | 0 | 66.26 | 10 | 1.7 |
| Control ADC-disulfide | 9 | 0 | 0 | 14.81 | 2 | 1.9 |
| methyl-PBD (“Control- | | | | | | |
| SSMe-PBD”) | | | | | | |
| Control ADC-disulfide-PBD | 9 | 0 | 0 | 66.26 | 10 | 1.7 |
| (“Control-SS-PBD”) | | | | | | |

*Vehicle = 20 mM histidine acetate, pH 5.5, 240 mM sucrose, 0.02% PS20;
n/a = not applicable.

In a 28 day time course with drug conjugates and doses as shown in Table 7, 10F4v3-PBD and 10F4v3-SSMe-PBD showed inhibition of tumor growth at 0.5 mg/kg in SCID mice with WSU-DLCL2 tumors compared to the vehicle and the control ADCs. See FIG. 7.

Furthermore, 2 mg/kg 10F4v3-SSMe-PBD showed almost complete tumor growth suppression. See FIG. 7. As shown in Table 2, 2 mice that received 2 μg/kg 10F4v3-SSMe-PBD and one mose that received 0.5 μg/kg 10F4v3-SSMe-PBD showed partial responses to the therapy.

In this study, the percent body weight change was determined in each dosage group. The results indicated that administration of the 10F4v3 ADCs did not cause a significant decrease in body weight during the study.

### H. Synthesis of Disulfide PBD Reagent

### (a) (S)-2-(methoxycarbonyl)-4-methylenepyrrolidinium chloride (3)

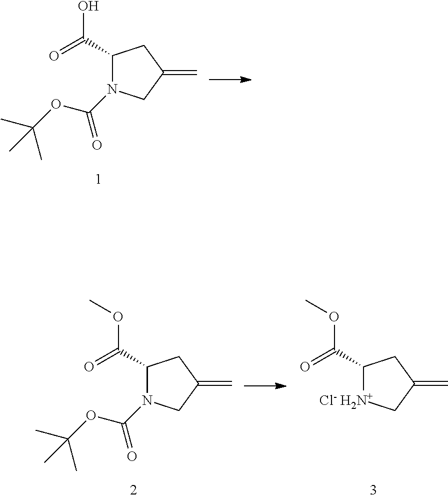

### (i) (S)-1-tert-butyl 2-methyl 4-methylenepyrrolidine-1,2-dicarboxylate (2)

Potassium carbonate (19.92 g, 14 mmol, 3 eq.) was added to a stirred solution of the carboxylic acid (1) (10.92 g, 48 mmol, 1 eq.) in DMF (270 mL). The resulting white suspension was stirred at room temperature for 30 minutes, at which point iodomethane (21.48 g, 9.5 mL, 151 mmol, 3.15 eq.) was added. The reaction mixture was allowed to stir at room temperature for 3 days. The DMF was removed by rotary evaporation under reduced pressure to afford a yellow residue which was partitioned between ethylacetate and water. The organic layer was separated and the aqueous phase was extracted with ethylacetate. The combined organic layers were washed with water, brine and dried over magnesium sulphate. The ethylacetate was removed by rotary evaporation under reduced pressure to give the crude product as a yellow oil. The crude product was purified by flash chromatography [85% n-hexane/15% ethylacetate] to afford the product as a colorless oil. (Known compound F Manfré et al., J. Org. Chem. 1992, 57, 2060-2065)

### (ii) (S)-2-(methoxycarbonyl)-4-methylenepyrrolidinium chloride (3)

A solution of 4 M hydrochloric acid in dioxane (63 mL, 254.4 mmol, 4.5 eq.) was added to the Boc protected C-ring fragment (2) (13.67 g, 56.6 mmol, 1 eq.) at room temperature. Effervescence was observed indicating liberation of CO₂ and removal of the Boc group. The product precipitated as a white solid and additional dioxane was added to facilitate stirring. The reaction mixture was allowed to stir for an hour and then diluted with ether. The precipitated product was collected by vacuum filtration and washed with additional ether. Air drying afforded the desired product as a white powder (9.42 g, 94%) (P Herdwijn et al., Canadian Journal of Chemistry. 1982, 60, 2903-7)

### (b) tert-butyl (5-((5-(5-amino-4-((S)-2-(((tert-butyldimethylsilyl)oxy)methyl)-4-methylenepyrrolidine-1-carbonyl)-2-methoxyphenoxy)pentyl)oxy)-24(S)-2-(((tert-butyldimethylsilyl)oxy)methyl)-4-methylenepyrrolidine-1-carbonyl)-4-methoxyphenyl)carbamate (9)

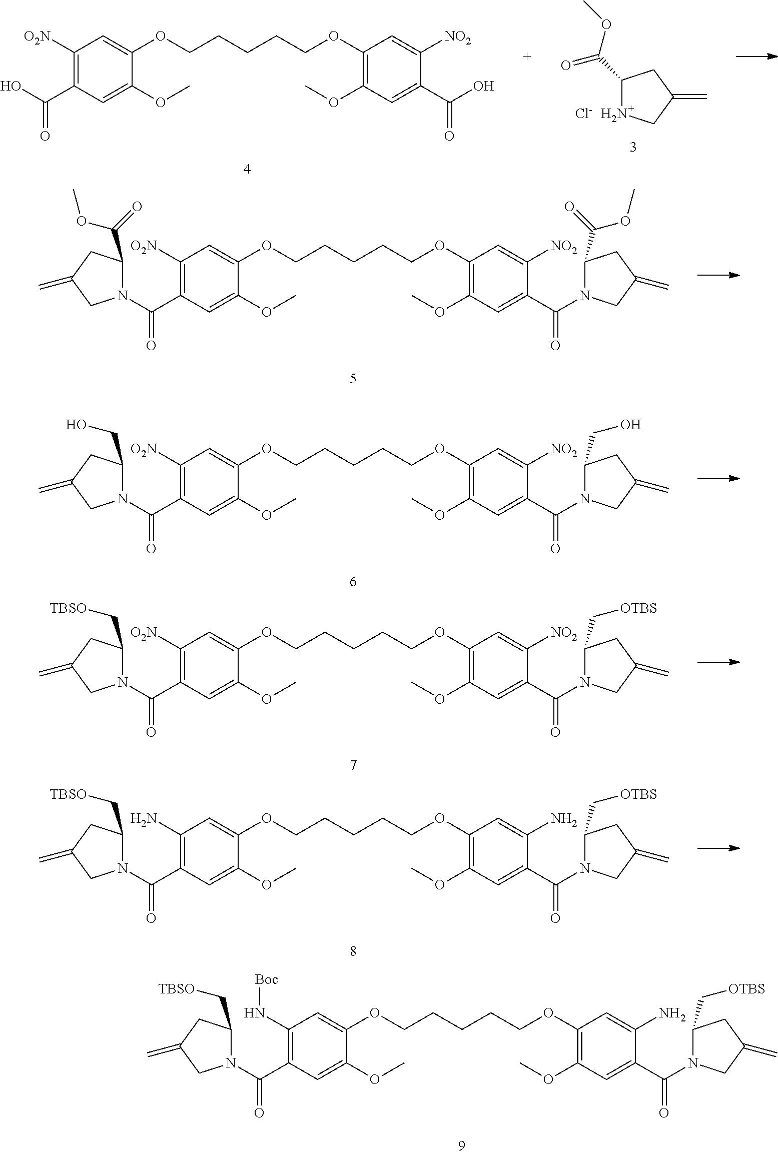

### (i) (S)-(4,4′-(pentane-1,5-diylbis(oxy))bis(5-methoxy-2-nitro-4,1-phenylene))bis(((S)-2-(methoxycarbonyl)-4-methylenepyrrolidin-1-yl)methanone) (5)

A catalytic amount of anhydrous DMF (0.5 mL) was added to a stirred suspension of oxalyl chloride (9.1 g, 6.25 mL, 71.7 mmol, 3 eq.) and dimer core (4) (11.82 g, 23.9 mmol, 1 eq.) in anhydrous DCM (180 mL) at room temperature. Vigorous effervescence was observed after the addition of DMF and the reaction mixture was allowed to stir for 18 h in a round bottom flask fitted with a calcium chloride drying tube. The resulting clear solution was evaporated under reduced pressure and the solid triturated with ether. The solid product was collected by vacuum filtration, washed with additional ether and dried in vacuo at 40° C. for 1.5 hours. This solid was then added portion wise to a suspension of the C-ring (3) (9.35 g, 52.6 mmol, 2.2 eq.) in TEA (12.08 g, 119.6 mmol, 5 eq.) and dry DCM (110 mL), maintaining the temperature between −40 and −50° C. with the aid of a dry ice/acetonitrile bath. The reaction mixture was allowed to stir at −40° C. for 1 hour and then allowed to warm to room temperature at which point LCMS indicated the complete consumption of the starting material. The reaction mixture was diluted with additional DCM and washed sequentially with aqueous hydrochloric acid (1M, 2×200 mL), saturated aqueous sodium bicarbonate (2×250 mL), water (250 mL), brine (250 mL), dried (MgSO₄). DCM was removed by rotary evaporation under reduced pressure to afford the product as a yellow foam (13.94 g, 79%). Analytical Data: RT 3.95 min; MS (ES⁺) m/z (relative intensity) 741 ([M+1]^{+.}, 100).

### (ii) (S)-(4,4′-(pentane-1,5-diylbis(oxy))bis(5-methoxy-2-nitro-4,1-phenylene))bis(((S)-2-(hydroxymethyl)-4-methylenepyrrolidin-1-yl)methanone) (6)

Solid lithium borohydride (0.093 g, 4.3 mmol, 3 eq.) was added in one portion to a solution of the ester (5) (1.05 g, 142 mmol, 1 eq.) in dry THF (10 mL) under a nitrogen atmosphere at 0° C. (ice bath). The reaction mixture was allowed to stir at 0° C. for 30 minutes and then allowed to warm to room temperature at which point precipitation of an orange gum was observed. The reaction mixture was allowed to stir at room temperature for a further 2 hours and then cooled in an ice bath and treated with water (20 mL) to give a yellow suspension. Hydrochloric acid (1M) was carefully added (vigorous effervescence!) until effervescence ceased. The reaction mixture was extracted with ethylacetate (4×50 mL) and the combined organic layers were washed with water (100 mL), brine (100 mL) and dried (MgSO₄). Ethylacetate was removed by rotary evaporation under reduced pressure to yield the product as a yellow foam (0.96 g, 99%). The reaction was repeated on a 12.4 g scale to yield 11.06 g of product (96%). Analytical Data: RT 3.37 min; MS (ES⁺) m/z (relative intensity) 685 ([M+H]^{+.}, 100).

### (iii) (S)-((pentane-1,5-diylbis(oxy))bis(5-methoxy-2-nitro-4,1-phenylene))bis(((S)-2-(((tert-butyldimethylsilyl)oxy)methyl)-4-methylenepyrrolidin-1-yl)methanone) (7)

A solution of bis-nitro alcohol (6) (7.94 g, 11.6 mmol, 1 eq), tert-butyldimethylsilylchloride (4.54 g, 30.15 mmol, 2.6 eq) and imidazole (4.1 g, 60.3 mmol, 5.2 eq) in anhydrous DMF (100 mL) under an argon atmosphere was stirred at room temperature for 3 hours. The reaction mixture was diluted with water (250 mL) and extracted with DCM (4×100 mL). The combined extracts were washed with water (200 mL), saturated brine (200 mL), dried (MgSO₄) and evaporated under reduced pressure. The residue was purified by flash column chromatography [50% ethylacetate/50% n-hexane to 100% ethylacetate in 10% increments] to afford the product as a yellow foam (10.0 g, 94%). Analytical Data: RT 4.57 min; MS (ES⁺) m/z (relative intensity) 913 ([M+H]^{+.}, 100).

### (iv) (S)-((pentane-1,5-diylbis(oxy))bis(2-amino-5-methoxy-4,1-phenylene))bis(((S)-2-(((tert-butyldimethylsilyl)oxy)methyl)-4-methylenepyrrolidin-1-yl)methanone) (8)

Formic acid solution (5% v/v, 15 mL) was added in one portion to a mixture of zinc powder (29.56 g, 0.45 mol, 40 eq.) and compound (7) (10.34 g, 11.32 mmol, 1 eq.) in ethylacetate/ethanol (80 mL/150 mL). An exotherm of 12° C. was observed. After 15 minutes the reaction mixture was filtered through celite washing with ethylacetate (excess). The filtrate was washed with saturated sodium bicarbonate (3×150 mL), water (200 mL), saturate brine (200 mL), dried (MgSO₄) and evaporated under reduced pressure. Purification by flash column chromatography [ethylacetate] gave the product as a white foam (8.09 g, 84%). Analytical Data: RT 4.43 min; MS (ES⁺) m/z (relative intensity) 853 ([M+H]^{+.}, 100).

(v) tert-butyl (5-((5-(5-amino-4-((S)-2-(((tert-butyldimethylsilyl)oxy)methyl)-4-methylenepyrrolidine-1-carbonyl)-2-methoxyphenoxy)pentyl)oxy)-2-((S)-2-(((tert-butyldimethylsilyl)oxy)methyl)-4-methylenepyrrolidine-1-carbonyl)-4-methoxyphenyl)carbamate (9)

A solution of the bis-aniline (8) (6.02 g, 7.1 mmol, 1 eq.) and di-t-butyl-dicarbonate (1.54 g, 7.1 mmol, 1 eq.) in anhydrous THF (50 mL) was heated at reflux for 16 hours. The solvent was evaporated under reduced pressure and the residue was purified by flash column chromatography [40% ethylacetate/60% n-hexane to 60% ethylacetate/40% n-hexane to 100% ethylacetate] to give the product as a white foam (3.22 g, 48%). Analytical Data: RT 4.27 min MS (ES⁺) m/z (relative intensity) 953 ([M+H]^{+.}, 100), MS (ES⁻) m/z (relative intensity) 951 ([M−H])⁻, 100).

### (c) (11S,11aS)-2-(pyridin-2-yldisulfanyl)ethyl 11-hydroxy-7-methoxy-8-((5-(((S)-7-methoxy-2-methylene-5-oxo-2,3,5,11a-tetrahydro-1H-pyrrolo[2,1-c][1,4]benzodiazepin-8-yl)oxy)pentyl)oxy)-2-methylene-5-oxo-2,3,11,11a-tetrahydro-1H-pyrrolo[2,1-c][1,4]benzodiazepine-10(5H)-carboxylate (14)

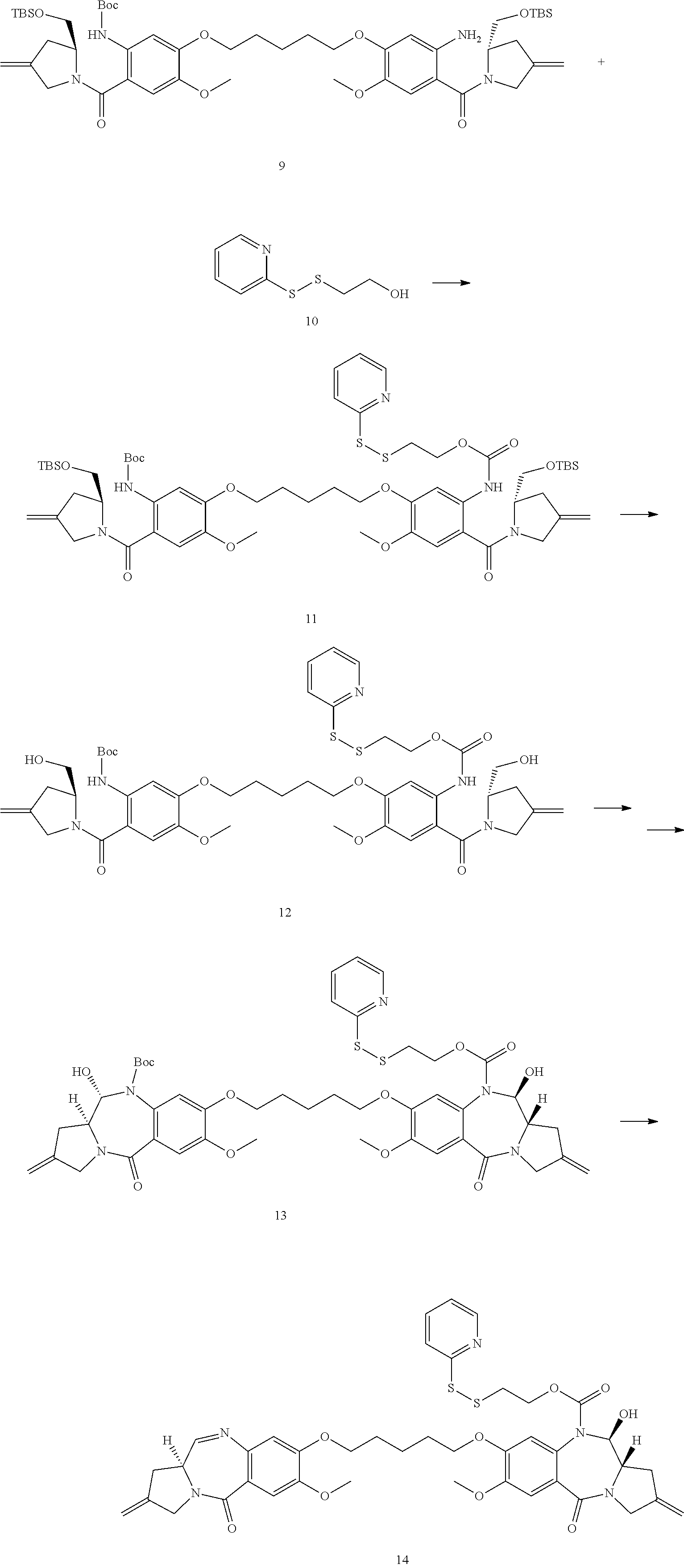

Compound 10 was prepared according to Jones et al, J. Am. Chem. Soc., 2006, 128, 6526-6527.

### (i) tert-butyl (2-(pyridin-2-yldisulfanyl)ethyl) ((S)-(pentane-1,5-diylbis(oxy))bis(2-((S)-2-(((tert-butyldimethylsilyl)oxy)methyl)-4-methylenepyrrolidine-1-carbonyl)-4-methoxy-5,1-phenylene))dicarbamate (11)

Triethylamine (0.25 g, 0.34 mL, 2.42 mmol, 2.2 eq.) was added to a stirred solution of the mono-Boc protected bis-aniline (9) (1.05 g, 1.1 mmol, 1.0 eq.) and triphosgene (0.117 g, 0.4 mmol, 0.36 eq.) in dry THF (10 mL) under an argon atmosphere at room temperature. The reaction mixture was heated to 40° C. and after 5 minutes a sample was treated with methanol and analysed by LCMS as the methyl carbamate. Analytical Data: RT 4.37 min MS (ES⁺) m/z (relative intensity) 1011 ([M+H]^{+.}, 100).

A solution of 2-(pyridin-2-yldisulfanyl)ethanol (10) (0.31 g, 1.65 mmol, 1.5 eq.) and triethylamine (0.17 g, 0.23 mL, 1.65 mmol, 1.5 eq.) in dry THF (10 mL) was added drop wise to the freshly prepared isocyanate. The reaction mixture was heated at 40° C. for 1.5 h after which time a further portion of triphosgene (0.058 g, 0.2 mmol, 0.18 eq.) was added. After a further 30 min the reaction mixture was allowed to cool, filtered to remove triethylamine hydrochloride and the filtrate was evaporated to dryness to afford the crude product as a yellow oil which was purified by flash column chromatography [60% n-hexane/40% ethylacetate changing to 55% n-hexane/45% ethylacetate] to give the desired product as a colourless oil (0.63 g, 49%). Analytical Data: RT 4.50 min; MS (ES⁺) m/z (relative intensity) 1166 ([M+H]^{+.}, 100), MS (ES⁻) m/z (relative intensity) 1164 ([M−H])^{-.}, 70).

### (ii) tert-butyl (2-(pyridin-2-yldisulfanyl)ethyl) ((S)-(pentane-1,5-diylbis(oxy))bis(2-((S)-2-(hydroxymethyl)-4-methylenepyrrolidine-1-carbonyl)-4-methoxy-5,1-phenylene))dicarbamate (12)

AcOH/H20 (3/1/) (8 mL) was added to a solution of compound (11) (0.37 g, 0.32 mmol, 1 eq) in THF (2 mL) and the resultant solution was stirred at room temperature for 18 h. The pH of the reaction mixture was adjusted to pH8 with saturated NaHCO₃ solution. The mixture was extracted with ethylacetate (3×100 mL) and the combined extracts were washed with saturated NaHCO₃ solution (100 mL), water (100 mL), saturated brine (100 mL), dried (MgSO₄) and evaporated under reduced pressure. Purification of the residue by flash column chromatography [gradient elution chloroform/methanol 0% to 5% in 1% increments] gave the product as a white foam (0.24 g, 81%). Analytical Data: RT 3.08 min; MS (ES⁺) m/z (relative intensity) 938 ([M+H]^{+.}, 100), MS (ES⁻) m/z (relative intensity) 936 ([M−H])^{-.}, 100).

### (iii) (11S,11aS)-tert-butyl 11-hydroxy-8-((5-(((11S,11aS)-11-hydroxy-7-methoxy-2-methylene-5-oxo-10-((2-(pyridin-2-yldisulfanyl)ethoxy)carbonyl)-2,3,5,10,11,11a-hexahydro-1H-pyrrolo[2,1-c][1,4]benzodiazepin-8-yl)oxy)pentyl)oxy)-7-methoxy-2-methylene-5-oxo-2,3,11,11a-tetrahydro-1H-pyrrolo[2,1-c][1,4]benzodiazepine-10(5H)-carboxylate (13)

A solution of DMSO (79 mg, 72 μL, 1.0 mmol, 4.4 eq) in DCM (5 mL) was added dropwise to a solution of oxalyl chloride (62 mg, 42 μL, 0.49 mmol, 2.15 eq.) in DCM (5 mL) under an argon atmosphere at −78° C. (dry ice/acetone). The solution was stirred at −78° C. for 15 minutes. A solution of compound (12) (0.214 g, 0.23 mmol, 1.0 eq.) in DCM (6 mL) was added dropwise and the mixture was stirred at −78° C. for 45 minutes. Triethylamine (0.23 g, 0.32 mL, 2.28 mmol, 10 eq.) was added and after 5 min the reaction mixture was allowed to reach room temperature. The reaction mixture was treated with saturated NH₄Cl solution (15 mL), the organic portion was separated and washed with 1M citric acid solution (3×50 mL), saturated NaHCO₃ solution (100 mL), water (100 mL), saturated brine (100 mL), dried (MgSO₄) and evaporated under reduced pressure to give a pale yellow oil. Purification by flash column chromatography gave the product as a white foam (68 mg, 32%). Analytical Data: RT 2.90 min; MS (ES⁺) m/z (relative intensity) 933 ([M+H]^{+.}, 50), MS (ES⁻) m/z (relative intensity) 935 ([M−H])^{-.}, 55).

### (iv) (11S,11aS)-2-(pyridin-2-yldisulfanyl)ethyl 11-hydroxy-7-methoxy-8-((5-(((S)-7-methoxy-2-methylene-5-oxo-2,3,5,11a-tetrahydro-1H-pyrrolo[2,1-c][1,4]benzodiazepin-8-yl)oxy)pentyl)oxy)-2-methylene-5-oxo-2,3,11,11a-tetrahydro-1H-pyrrolo[2,1-c][1,4]benzodiazepine-10(5H)-carboxylate (14)

A cold (ice bath) solution of 95% trifluoroacetic acid (1 mL) was added to compound 13 which had been cooled in an ice bath. The solution was stirred at 0° C. for 15 minutes when it was shown to be complete by LCMS. The reaction mixture was added dropwise to a mixture of ice and saturated NaHCO₃ solution to neutralise the trifluoroacetic acid solution. The mixture was extracted with DCM (4×50 mL) and the combined extracts were washed with saturated brine (100 mL), dried (MgSO₄) and evaporated under reduced pressure to give the product as a white foam (26 mg, 96%). Analytical Data: RT 2.72 min; MS (ES⁺) m/z (relative intensity) 816 ([M+H]^{+.}, 70), MS (ES⁻) m/z (relative intensity) 814 ([M−H])^{-.}, 40).

### I. Synthesis of Disulfide Methyl PBD Reagent

### (a) (R)-2-(pyridin-2-yldisulfanyl)propan-1-ol (18)

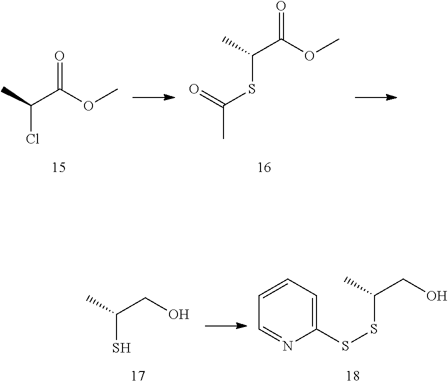

### (i) (R)-methyl 2-(acetylthio)propanoate (16)

Thioacetic acid (1.99 g, 1.86 mL, 26.1 mmol, 1.1 eq.) was added to a suspension of cesium carbonate (7.73 g, 23.72 mmol, 1.0 eq.) in dry DMF (40 mL). After 30 minutes (S)-methyl 2-chloropropanoate (15) was added and the mixture was allowed to stir at room temperature for 1 hour. The reaction mixture was partitioned between diethyl ether (150 mL) and water (150 mL); the water was separated and washed with a further portion of diethyl ether (150 mL). The combined organic portions were washed with water (6×100 mL), brine (200 mL), dried (MgSO₄) and evaporated under reduced pressure. Purification by flash column chromatography [10% ethylacetate/90% n-hexane] gave the product as a colourless oil (3.01 g, 82%). Analytical Data: RT 2.25 min; MS (ES⁺) m/z (relative intensity) 163 ([M+H]^{+.}, 10), 185([M+Na]^{+.}, 65); [α]^{t}_{d}=[+141]^{17.8° C.}_{d} (c, 2.26 CHCl₃).

### (ii) (R)-2-mercaptopropan-1-ol (17)

A solution of thioacetate (16) (0.57 g, 3.54 mmol, 1.0 eq.) in dry THF (10 mL) was added drop wise to a suspension of lithium aluminium hydride (0.54 g, 14.15 mmol, 4.0 eq.) in dry THF (20 mL) at reflux under an argon atmosphere. After 1 h the reaction mixture was cooled to 0° C. and 2M HCl was added drop wise maintaining the temperature below 30° C. until effervescence ceased. The resultant mixture was allowed to stir at room temperature for 1 hour then filtered through celite washing with THF (40 mL). The solvent was evaporated; the residue was re-dissolved in DCM and dried (MgSO₄). Evaporation of the DCM under reduced pressure followed by column chromatography of the residue [60% n-hexane/40% ethylacetate] gave the product as a pale yellow oil (0.193 g, 58%). Analytical Data: [α]^{t}_{d}=[−22]^{17.2° C.}_{d} (c, 0.972 CHCl₃).

### (iii) (R)-2-(pyridin-2-yldisulfanyl)propan-1-ol (18)

Sulfuryl chloride (1M in DCM, 2.0 mL, 2.0 mmol, 1.1 eq.) was added drop wise to a solution of 2-mercaptopyridine (0.2 g, 1.81 mmol, 1.0 eq.) in dry DCM (5 mL) at 0° C. under an argon atmosphere. The resultant solution was stirred at room temperature for 2 hours and the DCM was evaporated under reduced pressure to give a yellow solid. The solid was suspended in dry DCM (10 mL) and a solution of (R)-2-mercaptopropan-1-ol (17)(0.18 g, 1.95 mmol, 1.08 eq.) in dry DCM (5 mL) was added drop wise. The mixture was stirred at room temperature for 18 hours under an argon atmosphere. The reaction mixture was filtered and the filtrate was evaporated under reduced pressure to give a yellow gum. The gum was re-dissolved in water and the solution was basified with ammonium hydroxide solution, extracted with DCM (3×50 mL) and the combined extracts were washed with water (100 mL), brine (100 mL), dried (MgSO₄) and evaporated to give a yellow oil. Purification by flash column chromatography [80% n-hexane/20% ethylacetate to 60% n-hexane/40% ethylacetate in 5% increments] gave the product as a colourless oil (0.213 g, 59%). Analytical Data: RT 2.43 min; MS (ES⁺) m/z (relative intensity) 202 ([M+H]^{+.}, 50); [α]^{t}_{d}=[+273]^{26.2° C.}_{d} (c, 0.28 CHCl₃).

### (b) (11S,11aS)-(R)-2-(pyridin-2-yldisulfanyl)propyl 11-hydroxy-7-methoxy-8-((5-(((S)-7-methoxy-2-methylene-5-oxo-2,3,5,11a-tetrahydro-1H-pyrrolo[2,1-c][1,4]benzodiazepin-8-yl)oxy)pentyl)oxy)-2-methylene-5-oxo-2,3,11,11a-tetrahydro-1H-pyrrolo[2,1-c][1,4]benzodiazepine-10(5H)-carboxylate (22)

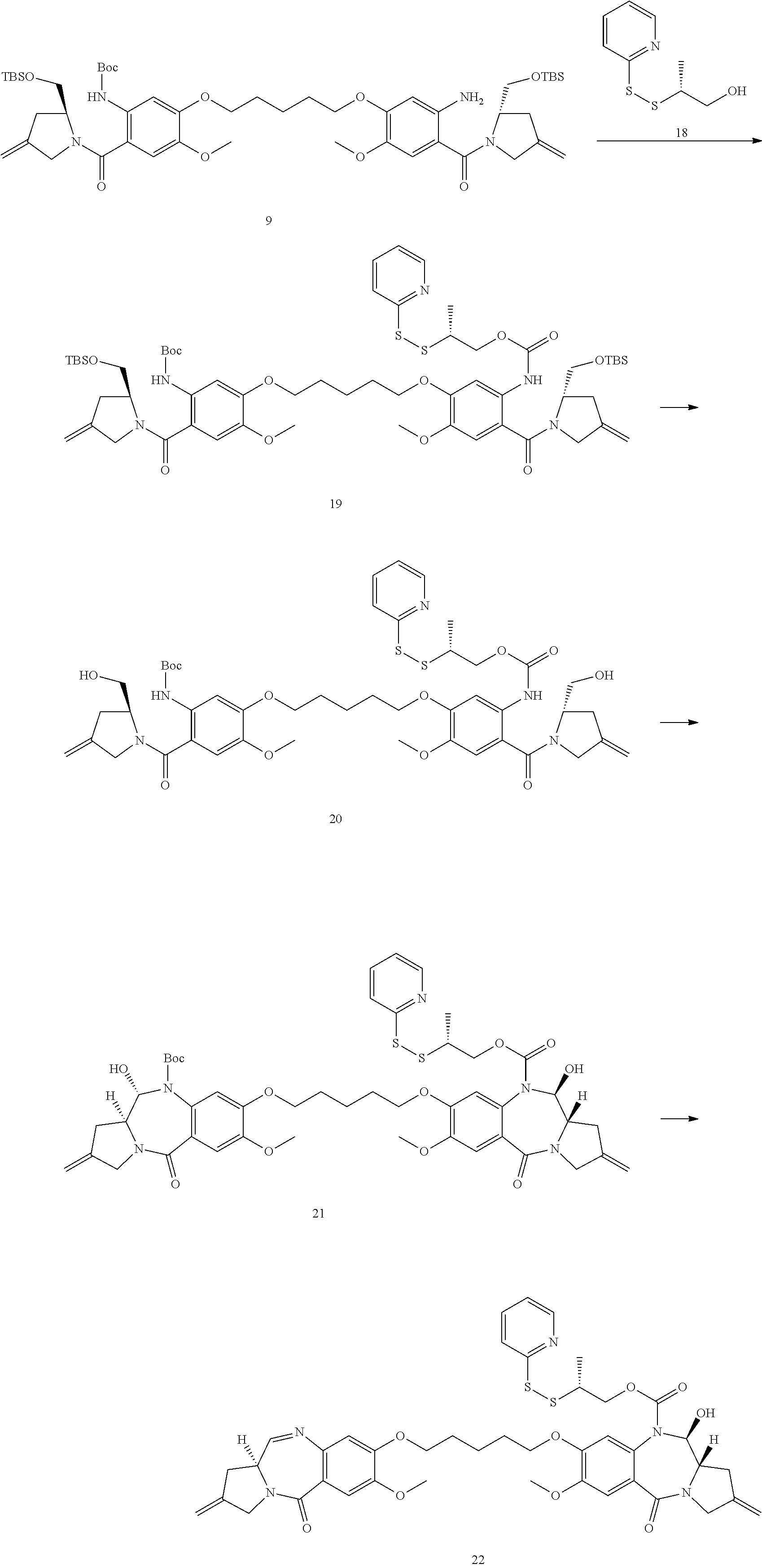

### (i) tert-butyl ((R)-2-(pyridin-2-yldisulfanyl)propyl) ((S)-(pentane-1,5-diylbis(oxy))bis(2-((S)-2-(((tert-butyldimethylsilyl)oxy)methyl)-4-methylenepyrrolidine-1-carbonyl)-4-methoxy-5,1-phenylene))dicarbamate (19)

Triethylamine (0.28 g, 0.39 mL, 2.8 mmol, 2.2 eq.) was added to a stirred solution of the mono-boc protected bis-aniline (9) (1.21 g, 1.27 mmol, 1.0 eq.) and triphosgene (0.136 g, 0.46 mmol, 0.36 eq.) in dry THF (15 mL) under an argon atmosphere at room temperature. The reaction mixture was heated to 40° C. and after 5 minutes a sample was treated with methanol and analysed by LCMS as the methyl carbamate. Analytical Data: RT 4.30 min MS (ES⁺) m/z (relative intensity) 1011 ([M+H]^{+.}, 100).

A solution of (R)-2-(pyridin-2-yldisulfanyl)propan-1-ol (18) (0.38 g, 1.91 mmol, 1.5 eq.) and triethylamine (0.19 g, 0.27 mL, 1.91 mmol, 1.5 eq.) in dry THF (10 mL) was added drop wise to the freshly prepared isocyanate. The reaction mixture was heated at 40° C. for 4 hours and then stirred at room temperature for 18 hours. The reaction mixture was filtered to remove triethylamine hydrochloride and the filtrate was evaporated to dryness to afford the crude product as a yellow oil which was purified by flash column chromatography [60% n-hexane/40% ethylacetate to 40% n-hexane/60% ethylacetate in 5% increments] to give the desired product as a white foam (0.75 g, 50%). Analytical Data: RT 4.50 min; MS (ES⁺) m/z (relative intensity) 1180 ([M+H]^{+.}, 60); [α]^{t}_{d}=[−18]^{21° C.}_{d} (c, 0.28 CHCl₃).

### (ii) tert-butyl ((R)-2-(pyridin-2-yldisulfanyl)propyl) ((S)-(pentane-1,5-diylbis(oxy))bis(2-((S)-2-(hydroxymethyl)-4-methylenepyrrolidine-1-carbonyl)-4-methoxy-5,1-phenylene))dicarbamate (20)

Acetic acid/H20 (3/1, 16 mL) was added to a solution, of the bis-silyl ether (19) (0.72 g, 0.61 mmol, 1 eq.) in THF (4 mL). The resultant solution was stirred at room temperature for 16 hours. The pH of the reaction mixture was adjusted to pH8 with saturated sodium bicarbonate solution. The mixture was extracted with ethylacetate (4×150 mL) and the combined extracts were washed with saturated sodium bicarbonate solution (2×150 mL), water (150 mL), brine (150 mL), dried (MgSO₄) and evaporated under reduced pressure. Purification by flash column chromatography gave the product as a white foam (0.56 g, 96%). Analytical Data: RT 3.15 min; MS (ES⁺) m/z (relative intensity) 953 ([M+H]^{+.}, 100); [α]^{t}_{d}=[−13.5]^{26° C.}_{d} (c, 0.22 CHCl₃).

### (iii) (11S,11aS)-tert-butyl 11-hydroxy-8-((5-(((11S,11aS)-11-hydroxy-7-methoxy-2-methylene-5-oxo-10-(((R)-2-(pyridin-2-yldisulfanyl)propoxy)carbonyl)-2,3,5,10,11,11a-hexahydro-1H-pyrrolo[2,1-c][1,4]benzodiazepin-8-yl)oxy)pentyl)oxy)-7-methoxy-2-methylene-5-oxo-2,3,11,11a-tetrahydro-1H-pyrrolo[2,1-c][1,4]benzodiazepine-10(5H)-carboxylate (21)

A solution of DMSO (91 mg, 83 μL, 1.16 mmol, 4.4 eq.) in anhydrous DCM (5 mL) was added drop-wise to a solution of oxalyl chloride (2.0M in DCM, 318 μL, 0.635 mmol, 2.4 eq.) in anhydrous DCM (5 mL). at −40° C. under an argon atmosphere. The solution was stirred at −40° C. for 15 minutes. A solution of the bis-alcohol (20) (0.252 g, 0.26 mmol, 1 eq.) in anhydrous DCM (10 mL) was added drop wise and the resultant mixture stirred at −40° C. for 45 minutes. During this time the temperature was allowed to reach −25° C. The temperature was lowered to −35° C. and triethylamine (0.27 g, 0.36 mL, 2.6 mmol, 10 eq.) was added drop wise. After 5 minutes the temperature was allowed to reach room temperature. The reaction mixture was diluted with DCM (50 mL) and extracted with 1M citric acid solution (3×150 mL), saturated sodium bicarbonate solution (150 mL), water (200 mL), brine (200 mL), dried (MgSO₄) and evaporated under reduced pressure to give a yellow foam. Purification by flash column chromatography [chloroform/methanol 0% to 2% in 0.5% increments] gave the product as a white foam (0.137 g, 53%). Analytical Data: RT 3.17 min; MS (ES⁺) m/z (relative intensity) 948 ([M+H]^{+.}, 100); [α]^{t}_{d}=+[170]^{26° C.}_{d} (c, 0.25 CHCl₃).

### (iv) (11S,11aS)-(R)-2-(pyridin-2-yldisulfanyl)propyl 11-hydroxy-7-methoxy-8-((5-(((S)-7-methoxy-2-methylene-5-oxo-2,3,5,11a-tetrahydro-1H-pyrrolo[2,1-c][1,4]benzodiazepin-8-yl)oxy)pentyl)oxy)-2-methylene-5-oxo-2,3,11,11a-tetrahydro-1H-pyrrolo[2,1-c][1,4]benzodiazepine-10(5H)-carboxylate (22)

A cold (ice bath) solution of 95% trifluoroacetic acid (8.5 mL) was added to compound (21) (0.221 g, 0.23 mmol, 1 eq.) which had been cooled in an ice bath. The solution was stirred at 0° C. for 25 minutes when it was shown to be complete by LCMS. The reaction mixture was added drop-wise to a mixture of ice and saturated sodium bicarbonate solution (200 mL) to neutralise the trifluoroacetic acid solution. The mixture was extracted with DCM (4×75 mL) and the combined extracts were washed with water (100 mL) saturated brine (100 mL), dried (MgSO₄) and evaporated under reduced pressure to give the crude product. Purification by flash column chromatography [chloroform/methanol 0% to 3% in 1% increments] gave the product as a white foam (0.192 g, 99%). Analytical Data: RT 3.00 min; MS (ES⁺) m/z (relative intensity) 830 ([M+H]^{+.}, 75); [α]^{t}_{d}=[+444]^{22° C.}_{d} (c, 0.26 CHCl₃).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

The material contained in the accompanying computer readable Sequence Listing identified as 2013-07-08_01146-0020_Sequence_Listing_ST25.txt, having a file creation date of Apr. 19, 2017, and file size of 72.9 KB, is incorporated herein by reference.

Table of Sequences

| SEQ | | |
|---|---|---|
| ID | | |
| NO | Description | Sequence |
| 1 | humIII variable | EVQLVESGGG LVQPGGSLRL SCAASGFTFS SYAMSWVRQA PGKGLEWVSV |
| | region sequence | ISGDGGSTYY ADSVKGRFTI SRDNSKNTLY LQMNSLRAED TAVYYCARGF |
| | | DYWGQGTLVT VSS |
| 2 | humκ1 variable | DIQMTQSPSS LSASVGDRVT ITCRASQSIS NYLAWYQQKP GKAPKLLIYA |
| | region sequence | ASSLESGVPS RFSGSGSGTD FTLTISSLQP EDFATYYCQQ YNSLPWTFGQ |
| | | GTKVEIK |
| 3 | mu10F4 heavy | QVQLQQSGPE LVKPGASVKI SCKASGYEFS RSWMNWVKQR PGQGREWIGR |
| | chain variable | IYPGDGDTNY SGKFKGKATL TADKSSSTAY MQLSSLTSVD SAVYFCARDG |
| | region | SSWDWYFDVW GAGTTVTVSS |
| 4 | mu10F4 light | DILMTQTPLS LPVSLGDQAS ISCRSSQSIV HSNGNTFLEW YLQKPGQSPK |
| | chain variable | LLIYKVSNRF SGVPDRFSGS GSGTDFTLKI SRVEAEDLGV YYCFQGSQFP |
| | region | YTFGGGTKVE IK |
| 5 | hu10F4v1 heavy | EVQLVESGGG LVQPGGSLRL SCAASGYEFS RSWMNWVRQA PGKGLEWVGR |
| | chain variable | IYPGDGDTNY SGKFKGRFTI SADTSKNTAY LQMNSLRAED TAVYYCARDG |
| | region | SSWDWYFDVW GQGTLVTVSS |
| 6 | hu10F4v1 light | DIQMTQSPSS LSASVGDRVT ITCRSSQSIV HSNGNTFLEW YQQKPGKAPK |
| | chain variable | LLIYKVSNRF SGVPSRFSGS GSGTDFTLTI SSLQPEDFAT YYCFQGSQFP |
| | region | YTFGQGTKVE IK |
| 7 | hu10F4v3 heavy | EVQLVESGGG LVQPGGSLRL SCAASGYEFS RSWMNWVRQA PGKGLEWVGR |
| | chain variable | IYPGDGDTNY SGKFKGRFTI SADTSKNTAY LQMNSLRAED TAVYYCARDG |
| | region | SSWDWYFDVW GQGTLVTVSS |
| 8 | hu10F4v3 light | DIQMTQSPSS LSASVGDRVT ITCRSSQSIV HSVGNTFLEW YQQKPGKAPK |
| | chain variable | LLIYKVSNRF SGVPSRFSGS GSGTDFTLTI SSLQPEDFAT YYCFQGSQFP |
| | region | YTFGQGTKVE IK |
| 9 | 10F4 HVR H1 | GYEFSRSWMN |
| 10 | 10F4 HVR H2 | GRIYPGDGDTNYSGKFKG |
| 11 | 10F4 HVR H3 | DGSSWDWYFDV |
| 12 | 10F4 HVR L1 | RSSQSIVHSNGNTFLE |
| 13 | 10F4 HVR L2 | KVSNRFS |
| 14 | 10F4 HVR L3 | FQGSQFPYT |
| 15 | 10F4 HVR L1 | RSSQSIVHSVGNTFLE |
| | N28V (10F4v3 | |
| | HVR L1) | |
| 16 | 10F4 HVR L1 | RSSQSIVHSAGNTFLE |
| | N28A | |
| 17 | 10F4 HVR L1 | RSSQSIVHSQGNTFLE |
| | N28Q | |
| 18 | 10F4 HVR L1 | RSSQSIVHSSGNTFLE |
| | N28S | |
| 19 | 10F4 HVR L1 | RSSQSIVHSDGNTFLE |
| | N28D | |
| 20 | 10F4 HVR L1 | RSSQSIVHSIGNTFLE |
| | N28I | |
| 21 | 10F4 HVR L1 | RSSQSIVHSNGITFLE |
| | N30A | |
| 22 | 10F4 HVR L1 | RSSQSIVHSNGQTFLE |
| | N30Q | |
| 45 | hu10F4v3 heavy | EVQLVESGGG LVQPGGSLRL SCAAS |
| | chain (HC) | |
| | framework (FR) 1 | |
| 46 | hu10F4v3 HC | WVRQA PGKGLEWV |
| | FR2 | |
| 47 | hul0F4v3 HC | RFTI SADTSKNTAY LQMNSLRAED TAVYYCAR |
| | FR3 | |
| 48 | hul0F4v3 HC | W GQGTLVTVSS |
| | FR4 | |
| 49 | hu10F4v3 light | DIQMTQSPSS LSASVGDRVT ITC |
| | chain (LC) | |
| | framework (FR) 1 | |
| 50 | hu10F4v3 LC | W YQQKPGKAPK LLIY |
| | FR2 | |
| 51 | hu10F4v3 LC | GVPSRFSGS GSGTDFTLTI SSLQPEDFAT YYC |
| | FR3 | |
| 52 | hu10F4v3 LC | FGQGTKVE IK |
| | FR4 | |
| 23 | hu10F4v3 light | DIQMTQSPSS LSASVGDRVT ITCRSSQSIV HSVGNTFLEW YQQKPGKAPK |
| | chain (Igκ) | LLIYKVSNRF SGVPSRFSGS GSGTDFTLTI SSLQPEDFAT YYCFQGSQFP |
| | | YTFGQGTKVE IKRTVAAPSV FIFPPSDEQL KSGTASVVCL LNNFYPREAK |
| | | VQWKVDNALQ SGNSQESVTE QDSKDSTYSL SSTLTLSKAD YEKHKVYACE |
| | | VTHQGLSSPV TKSFNRGEC |
| 24 | hu10F4v3 heavy | EVQLVESGGG LVQPGGSLRL SCAASGYEFS RSWMNWVRQA PGKGLEWVGR |
| | chain (IgG1) | IYPGDGDTNY SGKFKGRFTI aADTSKNTAY LQMNSLRAED TAVYYCARDG |
| | | SSWDWYFDVW GQGTLVTVSS ASTKGPSVFP LAPSSKSTSG GTAALGCLVK |
| | | DYFPEPVTVS WNSGALTSGV HTFPAVLQSS GLYSLSSVVT VPSSSLGTQT |
| | | YICNVNHKPS NTKVDKKVEP KSCDKTHTCP PCPAPELLGG PSVFLFPPKP |
| | | KDTLMISRTP EVTCVVVDVS HEDPEVKFNW YVDGVEVHNA KTKPREEQYN |
| | | STYRVVSVLT VLHQDWLNGK EYKCKVSNKA LPAPIEKTIS KAKGQPREPQ |
| | | VYTLPPSREE MTKNQVSLTC LVKGFYPSDI AVEWESNGQP ENNYKTTPPV |
| | | LDSDGSFFLY SKLTVDKSRW QQGNVFSCSV MHEALHNHYT QKSLSLSPGK |
| 25 | hu10F4V205C | DIQMTQSPSS LSASVGDRVT ITCRSSQSIV HSVGNTFLEW YQQKPGKAPK |
| | cysteine | LLIYKVSNRF SGVPSRFSGS GSGTDFTLTI SSLQPEDFAT YYCFQGSQFP |
| | engineered | YTFGQGTKVE IKRTVAAPSV FIFPPSDEQL KSGTASVVCL LNNFYPREAK |
| | light | VQWKVDNALQ SGNSQESVTE QDSKDSTYSL SSTLTLSKAD YEKHKVYACE |
| | chain (Igκ) | VTHQGLSSPC TKSFNRGEC |
| 26 | hu10F4v3 A118C | EVQLVESGGG LVQPGGSLRL SCAASGYEFS RSWMNWVRQA PGKGLEWVGR |
| | cysteine | IYPGDGDTNY SGKFKGRFTI aADTSKNTAY LQMNSLRAED TAVYYCARDG |
| | engineered | SSWDWYFDVW GQGTLVTVSS CSTKGPSVFP LAPSSKSTSG GTAALGCLVK |
| | heavy | DYFPEPVTVS WNSGALTSGV HTFPAVLQSS GLYSLSSVVT VPSSSLGTQT |
| | chain (IgG1) | YICNVNHKPS NTKVDKKVEP KSCDKTHTCP PCPAPELLGG PSVFLFPPKP |
| | | KDTLMISRTP EVTCVVVDVS HEDPEVKFNW YVDGVEVHNA KTKPREEQYN |
| | | STYRVVSVLT VLHQDWLNGK EYKCKVSNKA LPAPIEKTIS KAKGQPREPQ |
| | | VYTLPPSREE MTKNQVSLTC LVKGFYPSDI AVEWESNGQP ENNYKTTPPV |
| | | LDSDGSFFLY SKLTVDKSRW QQGNVFSCSV MHEALHNHYT QKSLSLSPGK |
| 27 | hu10F4v3 S400C | EVQLVESGGG LVQPGGSLRL SCAASGYEFS RSWMNWVRQA PGKGLEWVGR |
| | cysteine | IYPGDGDTNY SGKFKGRFTI aADTSKNTAY LQMNSLRAED TAVYYCARDG |
| | engineered | SSWDWYFDVW GQGTLVTVSS ASTKGPSVFP LAPSSKSTSG GTAALGCLVK |
| | heavy | DYFPEPVTVS WNSGALTSGV HTFPAVLQSS GLYSLSSVVT VPSSSLGTQT |
| | chainFc region | YICNVNHKPS NTKVDKKVEP KSCDKTHTCP PCPAPELLGG PSVFLFPPKP |
| | (IgG1) | KDTLMISRTP EVTCVVVDVS HEDPEVKFNW YVDGVEVHNA KTKPREEQYN |
| | | STYRVVSVLT VLHQDWLNGK EYKCKVSNKA LPAPIEKTIS KAKGQPREPQ |
| | | VYTLPPSREE MTKNQVSLTC LVKGFYPSDI AVEWESNGQP ENNYKTTPPV |
| | | LDCDGSFFLY SKLTVDKSRW QQGNVFSCSV MHEALHNHYT QKSLSLSPGK |
| 28 | Human CD22beta | mhllgpwlll lvleylafsd sskwvfehpe tlyawegacv wipctyrald |
| | precursor; | gdlesfilfh npeynkntsk fdgtrlyest kdgkvpseqk rvqflgdknk |
| | NP_0017622; | nctlsihpvh lndsgqlglr mesktekwme rihlnvserp fpphiqlppe |
| | signal sequence = | iqesqevtlt cllnfscygy piqlqwlleg vpmrqaavts tsltiksvft |
| | amino acids 1 to | rselkfspqw shhgkivtcq lqdadgkfls ndtvqlnvkh tpkleikvtp |
| | 19 | sdaivregds vtmtcevsss npeyttvswl kdgtslkkqn tftlnlrevt |
| | | kdqsgkyccq vsndvgpgrs eevflqvqya pepstvqilh spavegsqve |
| | | flcmslanpl ptnytwyhng kemqgrteek vhipkilpwh agtyscvaen |
| | | ilgtgqrgpg aeldvqyppk kvttviqnpm piregdtvtl scnynssnps |
| | | vtryewkphg aweepslgvl kiqnvgwdnt tiacarcnsw cswaspvaln |
| | | vqyaprdvry rkikplseih sgnsyslqcd fssshpkevq ffwekngrll |
| | | gkesqlnfds ispedagsys cwvnnsigqt askawtlevl yaprrlrvsm |
| | | spgdqvmegk satltcesda nppvshytwf dwnnqslphh sqklrlepvk |
| | | vqhsgaywcq gtnsvgkgrs plstltvyys petigrrvav glgsclaili |
| | | laicglklqr rwkrtqsqqg lgenssgqsf fvrnkkvrra plsegphslg |
| | | cynpmmedgi syttlrfpem niprtgdaes semqrpprtc ddtvtysalh |
| | | krqvgdyenv ipdfpedegi hyseliqfgv gerpqacienv dyvilkh |
| 29 | Human mature | d sskwvfehpe tiyawegacv wipctyraid gdiesfilfh npeynkntsk |
| | CD22beta, | fdgtrlyest kdgkvpseqk rvqfigdknk nctisihpvh indsgqigir |
| | without signal | mesktekwme rihinvserp fpphiqippe iqesqevtit clinfscygy |
| | sequence; amino | piqlqwileg vpmrqaavts tsitiksvft rselkfspqw shhgkivtcq |
| | acids 20 to 847 | iqdadgkfis ndtvqinvkh tpkieikvtp sdaivregds vtmtcevsss |
| | | npeyttvswi kdgtsikkqn tftinirevt kdqsgkyccq vsndvgpgrs |
| | | eevfiqvqya pepstvqiih spavegsqve ficmslanpi ptnytwyhng |
| | | kemqgrteek vhipkiipwh agtyscvaen ilgtgqrgpg aeldvqyppk |
| | | kvttviqnpm piregdtvti scnynssnps vtryewkphg aweepsigvi |
| | | kiqnvgwdnt tiacarcnsw cswaspvain vqyaprdvry rkikpiseih |
| | | sgnsysiqcd fssshpkevq ffwekngril gkesqinfds ispedagsys |
| | | cwvnnsigqt askawtievi yaprrirvsm spgdqvmegk satitcesda |
| | | nppvshytwf dwnnqslphh sqkirlepvk vqhsgaywcq gtnsvgkgrs |
| | | pistitvyys petigrrvav gigsciaili laicgikiqr rwkrtqsqqg |
| | | igenssgqsf fvrnkkvrra pisegphsig cynpmmedgi syttirfpem |
| | | niprtgdaes semqrpprtc ddtvtysalh krqvgdyenv ipdfpedegi |
| | | hyseliqfgv gerpqaqenv dyvilkh |
| 30 | Human | MHLLGPWLLL LVLEYLAFSD SSKWVFEHPE TLYAWEGACV WIPCTYRALD |
| | CD22alpha | GDLESFILFH NPEYNKNTSK FDGTRLYEST KDGKVPSEQK RVQFLGDKNK |
| | precursor; | NCTLSIHPVH LNDSGQLGLR MESKTEKWME RIHLNVSERP FPPHIQLPPE |
| | NP_001172030.1; | IQESQEVTLT CLLNFSCYGY PIQLQWLLEG VPMRQAAVTS TSLTIKSVFT |
| | signal sequence = | RSELKFSPQW SHHGKIVTCQ LQDADGKFLS NDTVQLNVKH PPKKVTTVIQ |
| | amino acids 1 to | NPMPIREGDT VTLSCNYNSS NPSVTRYEWK PHGAWEEPSL GVLKIQNVGW |
| | 19 | DNTTIACAAC NSWCSWASPV ALNVQYAPRD VRVRKIKPLS EIHSGNSVSL |
| | | QCDFSSSHPK EVQFFWEKNG RLLGKESQLN FDSISPEDAG SYSCWVNNSI |
| | | GQTASKAWTL EVLYAPRRLR VSMSPGDQVM EGKSATLTCE SDANPPVSHY |
| | | TWFDWNNQSL PYHSQKLRLE PVKVQHSGAY WCQGTNSVGK GRSPLSTLTV |
| | | YYSPETIGRR VAVGLGSCLA ILILAICGLK LQRRWKRTQS QQGLQENSSG |
| | | QSFFVRNKKV RRAPLSEGPH SLGCYNPMME DGISYTTLRF PEMNIPRTGD |
| | | AESSEMQRPP PDCDDTVTYS ALHKRQVGDY ENVIPDFPED EGIHYSELIQ |
| | | FGVGERPQAQ ENVDYVILKH |
| 31 | Human mature | D SSKWVFEHPE TLYAWEGACV WIPCTYRALD GDLESFILFH NPEYNKNTSK |
| | CD22alpha, | FDGTRLYEST KDGKVPSEQK RVQFLGDKNK NCTLSIHPVH LNDSGQLGLR |
| | without signal | MESKTEKWME RIHLNVSERP FPPHIQLPPE IQESQEVTLT CLLNFSCYGY |
| | sequence; amino | PIQLQWLLEG VPMRQAAVTS TSLTIKSVFT RSELKFSPQW SHHGKIVTCQ |
| | acids 20 to 670 | LQDADGKFLS NDTVQLNVKH PPKKVTTVIQ NPMPIREGDT VTLSCNYNSS |
| | | NPSVTRYEWK PHGAWEEPSL GVLKIQNVGW DNTTIACAAC NSWCSWASPV |
| | | ALNVQYAPRD VRVRKIKPLS EIHSGNSVSL |
| | | QCDFSSSHPK EVQFFWEKNG RLLGKESQLN FDSISPEDAG SYSCWVNNSI |
| | | GQTASKAWTL EVLYAPRRLR VSMSPGDQVM EGKSATLTCE SDANPPVSHY |
| | | TWFDWNNQSL PYHSQKLRLE PVKVQHSGAY WCQGTNSVGK GRSPLSTLTV |
| | | YYSPETIGRR VAVGLGSCLA ILILAICGLK LQRRWKRTQS QQGLQENSSG |
| | | QSFFVRNKKV RRAPLSEGPH SLGCYNPMME DGISYTTLRF PEMNIPRTGD |
| | | AESSEMQRPP PDCDDTVTYS ALHKRQVGDY ENVIPDFPED EGIHYSELIQ |
| | | FGVGERPQAQ ENVDYVILKH |
| 32 | Anti-CD79b | GYTFSSYWIE |
| | huMA79bv28 | |
| | HVR H1 | |
| 33 | Anti-CD79b | GEILPGGGDTNYNETFKG |
| | huMA79bv28 | |
| | HVR H2 | |
| 34 | Anti-CD79b | TRRVPIPLDY |
| | huMA79bv28 | |
| | HVR H3 | |
| 35 | Anti-CD79b | KASQSVDYEDSFLN |
| | huMA79bv28 | |
| | HVR L1 | |
| 36 | Anti-CD79b | AASNLES |
| | huMA79bv28 | |
| | HVR L2 | |
| 37 | Anti-CD79b | QQSNEDPLT |
| | huMA4A79bv28 | |
| | ENR L3 | |
| 38 | Anti-CD79b | EVQLVESGGG LVQPGGSLRL SCAASGYTFS SYWIEWVRQA PGKGLEWIGE |
| | MA4A79bv28 | ILPGGGDTNY NEIFKGRATF aADTSKNTAY LQMNSLRAED TAVYYCTRRV |
| | heavy chain | PIRLDYWGQG TLVTVSS |
| | variable region | |
| 39 | Anti-CD79b | DIQLTQSPSS LSASVGDRVT ITCKASQSVD YEGDSFLNWY QQKPGKAPKL |
| | MA4A79bv28 | LIYAASNLES GVPSRFSGSG SGTDFTLTIS SLQPEDFATY YCQQSNEDPL |
| | light chain | TFGQGTKVEI KR |
| | variable region | |
| 40 | Anti-CD79b | EVQLVESGGG LVQPGGSLRL SCAASGYTFS SYWIEWVRQA PGKGLEWIGE |
| | MA4A79bv28 | ILPGGGDTNY NEIFKGRATF aADTSKNTAY LQMNSLRAED TAVYYCTRRV |
| | A118C cysteine | PIRLDYWGQG TLVTVSSCST KGPSVFPLAP SSKSTSGGTA ALGCLVKDYF |
| | engineered heavy | PEPVTVSWNS GALTSGVHTF PAVLQSSGLY SLSSVVTVPS SSLGTQTYIC |
| | chain (IgG1) | NVNHKPSNTK VDKKVEPKSC DKTHTCPPCP APELLGGPSV FLFPPKPKDT |
| | | LMISRTPEVT CVVVDVSHED PEVKFNWYVD GVEVHNAKTK PREEQYNSTY |
| | | RVVSVLTVLH QDWLNGKEYK CKVSNKALPA PIEKTISKAK GQPREPQVYT |
| | | LPPSREEMTK NQVSLTCLVK GFYPSDIAVE WESNGQPENN YKTTPPVLDS |
| | | DGSFFLYSKL TVDKSRWQQG NVFSCSVMHE ALHNHYTQKS LSLSPG |
| 41 | Anti-CD79b | DIQLTQSPSS LSASVGDRVT ITCKASQSVD YEGDSFLNWY QQKPGKAPKL |
| | MA4A79bv28 | LIYAASNLES GVPSRFSGSG SGTDFTLTIS SLQPEDFATY YCQQSNEDPL |
| | light chain (Igκ) | TFGQGTKVEI KRTVAAPSVF IFPPSDEQLK SGTASVVCLL NNFYPREAKV |
| | | QWKVDNALQS GNSQESVTEQ DSKDSTYSLS STLTLSKADY EKHKVYACEV |
| | | THQGLSSPVT KSFNRGEC |
| 42 | Anti-CD79b | DIQLTQSPSS LSASVGDRVT ITCKASQSVD YEGDSFLNWY QQKPGKAPKL |
| | MA4A79bv28 | LIYAASNLES GVPSRFSGSG SGTDFTLTIS SLQPEDFATY YCQQSNEDPL |
| | V205C cysteine | TFGQGTKVEI KRTVAAPSVF IFPPSDEQLK SGTASVVCLL NNFYPREAKV |
| | engineered light | QWKVDNALQS GNSQESVTEQ DSKDSTYSLS STLTLSKADY EKHKVYACEV |
| | chain (Igκ) | THQGLSSPCT KSFNRGEC |
| 44 | Anti-CD79b | EVQLVESGGG LVQPGGSLRL SCAASGYTFS SYWIEWVRQA PGKGLEWIGE |
| | MA4A79bv28 | ILPGGGDTNY NEIFKGRATF aADTSKNTAY LQMNSLRAED TAVYYCTRRV |
| | heavy chain | PIRLDYWGQG TLVTVSSAST KGPSVFPLAP SSKSTSGGTA ALGCLVKDYF |
| | (IgG1) | PEPVTVSWNS GALTSGVHTF PAVLQSSGLY SLSSVVTVPS SSLGTQTYIC |
| | | NVNHKPSNTK VDKKVEPKSC DKTHTCPPCP APELLGGPSV FLFPPKPKDT |
| | | LMISRTPEVT CVVVDVSHED PEVKFNWYVD GVEVHNAKTK PREEQYNSTY |
| | | RVVSVLTVLH QDWLNGKEYK CKVSNKALPA PIEKTISKAK GQPREPQVYT |
| | | LPPSREEMTK NQVSLTCLVK GFYPSDIAVE WESNGQPENN YKTTPPVLDS |
| | | DGSFFLYSKL TVDKSRWQQG NVFSCSVMHE ALHNHYTQKS LSLSPG |
| 43 | Anti-CD79b | EVQLVESGGG LVQPGGSLRL SCAASGYTFS SYWIEWVRQA PGKGLEWIGE |
| | MA4A79bv28 | ILPGGGDTNY NEIFKGRATF aADTSKNTAY LQMNSLRAED TAVYYCTRRV |
| | S400C cysteine | PIRLDYWGQG TLVTVSSAST KGPSVFPLAP SSKSTSGGTA ALGCLVKDYF |
| | engineered heavy | PEPVTVSWNS GALTSGVHTF PAVLQSSGLY SLSSVVTVPS SSLGTQTYIC |
| | chain (IgG1) | NVNHKPSNTK VDKKVEPKSC DKTHTCPPCP APELLGGPSV FLFPPKPKDT |
| | | LMISRTPEVT CVVVDVSHED PEVKFNWYVD GVEVHNAKTK PREEQYNSTY |
| | | RVVSVLTVLH QDWLNGKEYK CKVSNKALPA PIEKTISKAK GQPREPQVYT |
| | | LPPSREEMTK NQVSLTCLVK GFYPSDIAVE WESNGQPENN YKTTPPVLDC |
| | | DGSFFLYSKL TVDKSRWQQG NVFSCSVMHE ALHNHYTQKS LSLSPGK |

1-45. (canceled)

46. An immunoconjugate of formula Ab-(L-D)p, wherein:
(a) Ab is an antibody that binds CD22;
(b) L is a linker;
(c) D is a cytotoxic agent; and
(d) p ranges from 1-8;
wherein D is a pyrrolobenzodiazepine of Formula A: 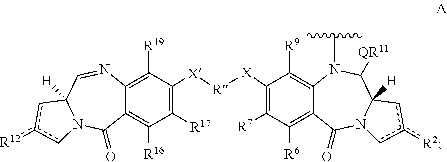
wherein the wavy line indicates a covalent attachment site to the linker;
the dotted lines indicate the optional presence of a double bond between C1 and C2 or C2 and C3;
R2 is independently selected from H, OH, ═O, ═CH₂, CN, R, OR, ═CH—RD, ═C(RD)2, O—SO2-R, CO2R and COR, and optionally further selected from halo or dihalo, wherein RD is independently selected from R, CO2R, COR, CHO, CO2H, and halo;
R6 and R9 are independently selected from H, R, OH, OR, SH, SR, NH2, NHR, NRR′, NO2, Me3 Sn and halo;
R7 is independently selected from H, R, OH, OR, SH, SR, NH2, NHR, NRR′, NO2, Me3Sn and halo;
Q is independently selected from O, S and NH;
R11 is either H, or R or, where Q is O, SO3M, where M is a metal cation;
R and R′ are each independently selected from optionally substituted C1-12 alkyl, C3-20 heterocyclyl and C5-20 aryl groups, and optionally in relation to the group NRR′, R and R′ together with the nitrogen atom to which they are attached form an optionally substituted 4-, 5-, 6- or 7-membered heterocyclic ring;
R12, R16, R19 and R17 are as defined for R2, R6, R9 and R7 respectively;
R″ is a C3-12 alkylene group, which chain may be interrupted by one or more heteroatoms and/or aromatic rings that are optionally substituted; and
X and X′ are independently selected from O, S and N(H); and wherein the antibody comprises:
(i) HVR-H1 comprising the amino acid sequence of SEQ ID NO: 9, (ii) HVR-H2 comprising the amino acid sequence of SEQ ID NO: 10, (iii) HVR-H3 comprising the amino acid sequence of SEQ ID NO: 11, (iv) HVR-L1 comprising an amino acid sequence selected from SEQ ID NOs: 12 and 15 to 22, (v) HVR-L2 comprising the amino acid sequence of SEQ ID NO: 13, and (vi) HVR-L3 comprising the amino acid sequence of SEQ ID NO: 14.

47. The immunoconjugate of claim 46, wherein the antibody comprises HVR-L1 comprising the amino acid sequence of SEQ ID NO: 15.

48. The immunoconjugate of claim 46, wherein the antibody comprises:
a) a VH sequence having at least 95% sequence identity to the amino acid sequence of SEQ ID NO: 7; or
b) a VL sequence having at least 95% sequence identity to the amino acid sequence of SEQ ID NO: 6 or SEQ ID NO: 8; or
c) a VH sequence as in (a) and a VL sequence as in (b).

49. The immunoconjugate of claim 46, comprising a VH sequence having the amino acid sequence of SEQ ID NO: 7 and a VL sequence having the amino acid sequence of SEQ ID NO: 6 or SEQ ID NO:8.

50. The immunoconjugate of claim 46, wherein the antibody is an IgG1, IgG2a or IgG2b antibody.

51. The immunoconjugate of claim 46, wherein D has the structure selected from: 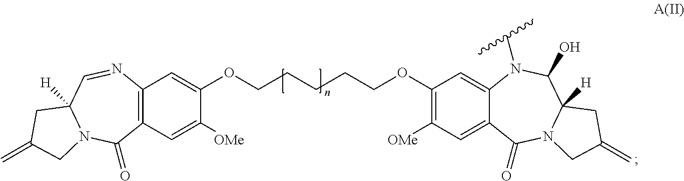
wherein n is 0 or 1; 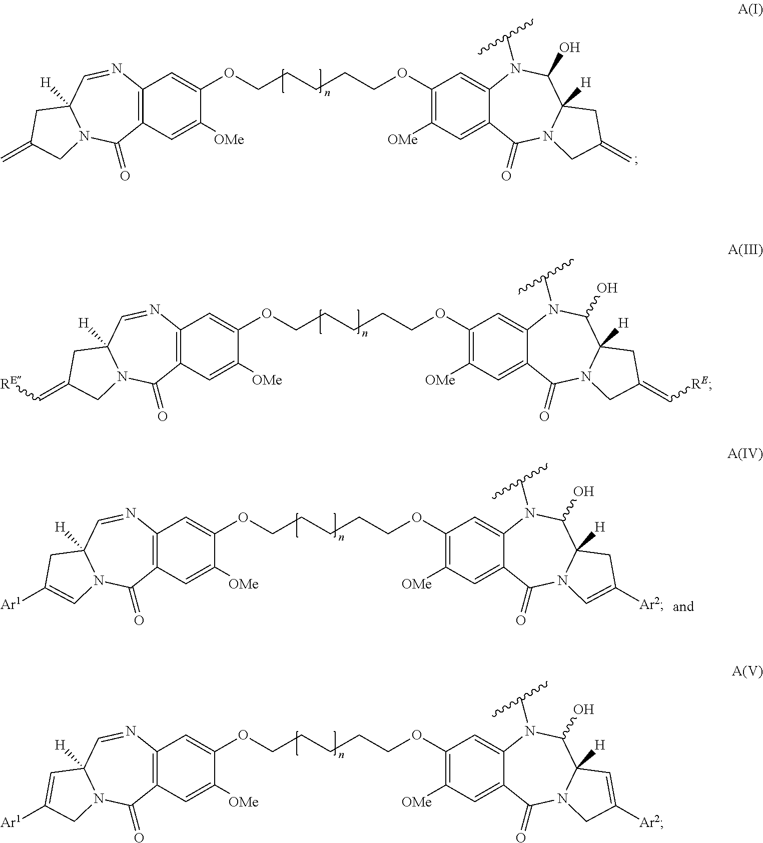
wherein R^{E} and R^{E″} are each independently selected from H or RD, wherein RD is independently selected from R, CO2R, COR, CHO, CO2H, and halo;
wherein Ar1 and Ar2 are each independently optionally substituted C5-20 aryl; and
wherein n is 0 or 1.

52. The immunoconjugate of claim 46, wherein D is a pyrrolobenzodiazepine of Formula B: 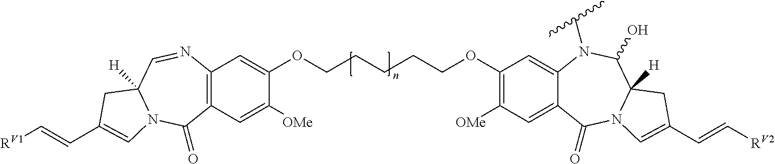
wherein the horizontal wavy line indicates the covalent attachment site to the linker;
RV1 and RV2 are independently selected from H, methyl, ethyl, phenyl, fluoro-substituted phenyl, and C5-6 heterocyclyl; and
n is 0 or 1.

53. The immunoconjugate of claim 46, wherein the linker is cleavable by a protease.

54. The immunoconjugate of claim 53, wherein the linker comprises a val-cit dipeptide or a Phe-Lys dipeptide.

55. The immunoconjugate of claim 46 having the formula

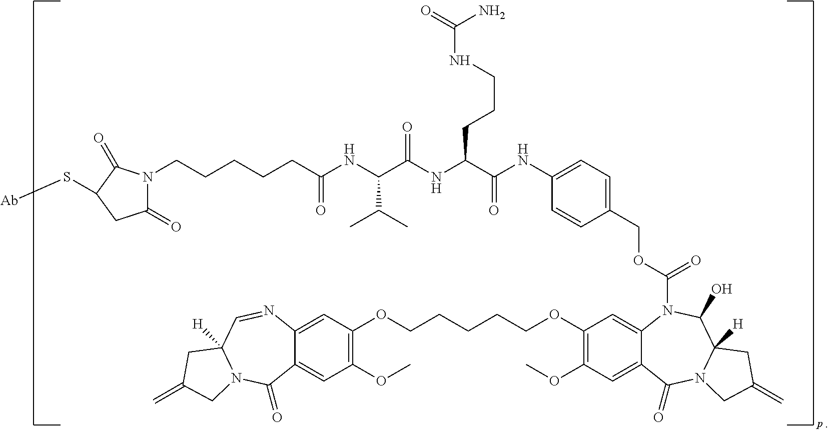

56. The immunoconjugate of claim 46, wherein p ranges from 1-3.

57. The immunoconjugate of claim 46, comprising the structure D: 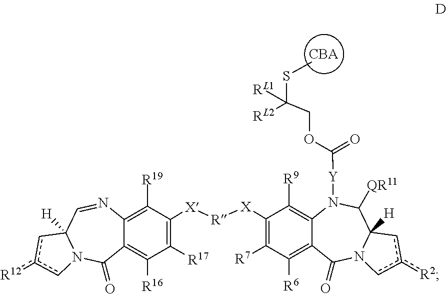
wherein CBA represents the antibody (Ab); RL1 and RL2 are each independently selected from H and methyl, or together with the carbon atom to which they are bound form a cyclopropylene group; and Y is selected from a single bond, (a1), and (a2): 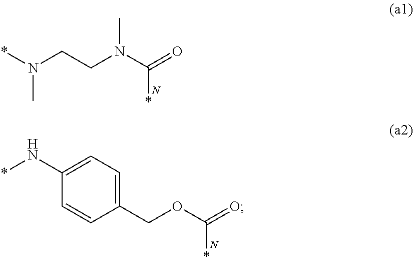
wherein N shows where the group binds to the N10 of the PBD moiety.

58. The immunoconjugate of claim 57, comprising a structure selected from:

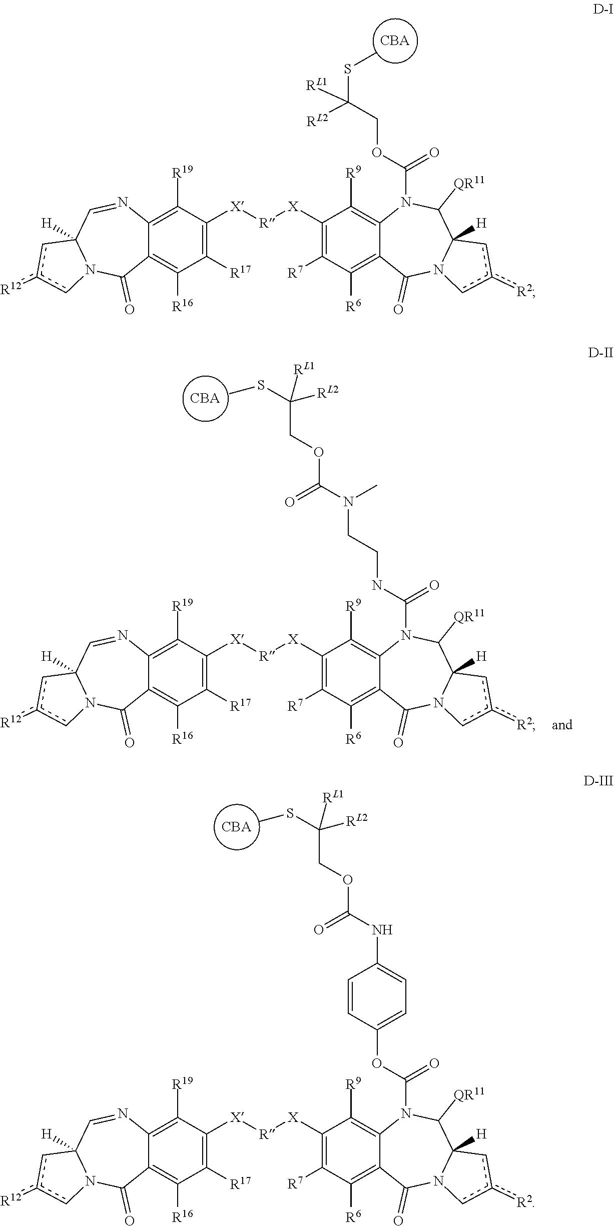

59. The immunoconjugate of claim 57, comprising the structure: 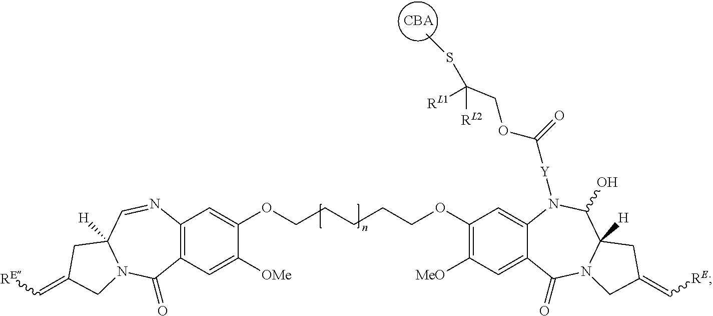
wherein R^{E} and R^{E″} are each independently selected from H and RD; or 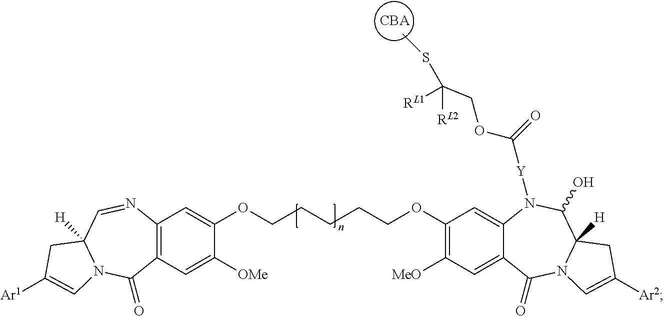
wherein Ar1 and Ar2 are each independently optionally substituted C5-20 aryl.

60. The immunoconjugate of claim 59, wherein Ar1 and Ar2 are each independently selected from optionally substituted phenyl, furanyl, thiophenyl and pyridyl.

61. The immunoconjugate of claim 57, comprising the structure: 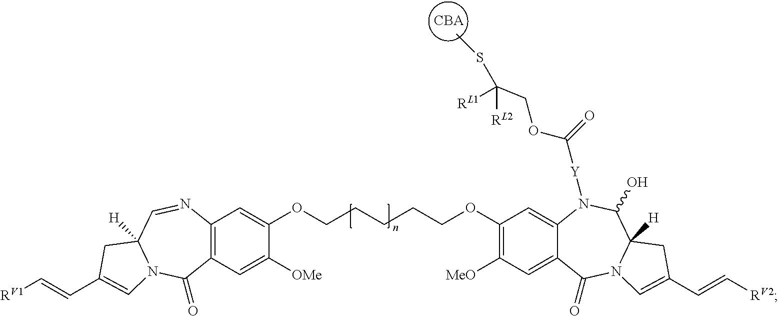
wherein RV1 and RV2 are each independently selected from H, methyl, ethyl, optionally substituted phenyl, and C5-6 heterocyclyl.

62. The immunoconjugate of claim 61, wherein RV1 and RV2 are each independently selected from H, phenyl, and 4-fluorophenyl.

63. An immunoconjugate having a formula selected from: 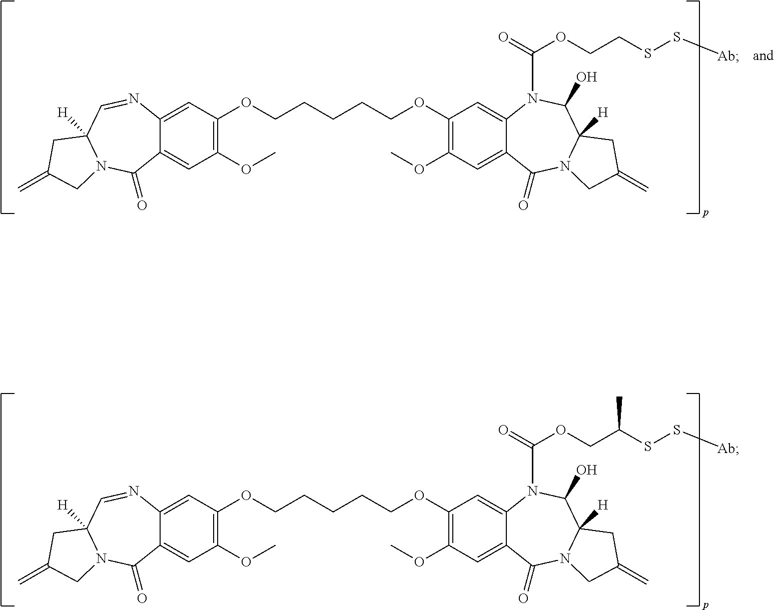
wherein Ab is an antibody comprising (i) HVR-H1 comprising the amino acid sequence of SEQ ID NO: 9, (ii) HVR-H2 comprising the amino acid sequence of SEQ ID NO: 10, (iii) HVR-H3 comprising the amino acid sequence of SEQ ID NO: 11, (iv) HVR-L1 comprising the amino acid sequence of SEQ ID NO: 15, (v) HVR-L2 comprising the amino acid sequence of SEQ ID NO: 13, and (vi) HVR-L3 comprising the amino acid sequence of SEQ ID NO: 14; and wherein p ranges from 1 to 3.

64. The immunoconjugate of claim 46 having the formula: 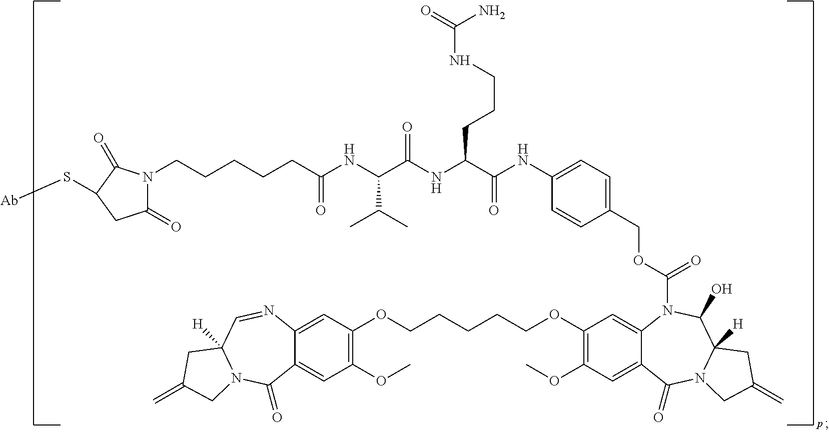
wherein Ab is an antibody comprising (i) HVR-H1 comprising the amino acid sequence of SEQ ID NO: 9, (ii) HVR-H2 comprising the amino acid sequence of SEQ ID NO: 10, (iii) HVR-H3 comprising the amino acid sequence of SEQ ID NO: 11, (iv) HVR-L1 comprising the amino acid sequence of SEQ ID NO: 15, (v) HVR-L2 comprising the amino acid sequence of SEQ ID NO: 13, and (vi) HVR-L3 comprising the amino acid sequence of SEQ ID NO: 14; and wherein p ranges from 1 to 3.

65. The immunoconjugate of claim 64, wherein the antibody comprises a VH sequence of SEQ ID NO: 7 and a VL sequence of SEQ ID NO: 8.

66. The immunoconjugate of claim 64, wherein the antibody comprises a heavy chain of SEQ ID NO: 26 and a light chain of SEQ ID NO: 23.

67. The immunoconjugate of claim 46, wherein the antibody is a monoclonal antibody.

68. The immunoconjugate of claim 46, wherein the antibody is a human, humanized, or chimeric antibody.

69. The immunoconjugate of claim 46, wherein the antibody is an antibody fragment that binds CD22.

70. The immunoconjugate of claim 46, wherein the antibody binds human CD22.

71. The immunoconjugate of claim 70, wherein human CD22 has the sequence of SEQ ID NO: 28 or SEQ ID NO: 29.

72. A pharmaceutical composition comprising the immunoconjugate of claim 46 and a pharmaceutically acceptable carrier.

73. The pharmaceutical composition of claim 72, further comprising an additional therapeutic agent.

74. A method for treating an individual having a CD22-positive cancer, the method comprising administering to the individual an effective amount of an immunoconjugate of formula Ab-(L-D)p, wherein:
(a) Ab is an antibody that binds CD22;
(b) L is a linker;
(c) D is a cytotoxic agent; and
(d) p ranges from 1-8;
wherein D is a pyrrolobenzodiazepine of Formula A: 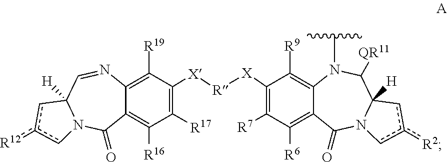
wherein the wavy line indicates a covalent attachment site to the linker;
the dotted lines indicate the optional presence of a double bond between C1 and C2 or C2 and C3;
R2 is independently selected from H, OH, ═O, ═CH₂, CN, R, OR, ═CH—RD, ═C(RD)2, O—SO2-R, CO2R and COR, and optionally further selected from halo or dihalo, wherein RD is independently selected from R, CO2R, COR, CHO, CO2H, and halo;
R6 and R9 are independently selected from H, R, OH, OR, SH, SR, NH2, NHR, NRR′, NO2, Me3 Sn and halo;
R7 is independently selected from H, R, OH, OR, SH, SR, NH2, NHR, NRR′, NO2, Me3Sn and halo;
Q is independently selected from O, S and NH;
R11 is either H, or R or, where Q is O, SO3M, where M is a metal cation;
R and R′ are each independently selected from optionally substituted C1-12 alkyl, C3-20 heterocyclyl and C5-20 aryl groups, and optionally in relation to the group NRR′, R and R′ together with the nitrogen atom to which they are attached form an optionally substituted 4-, 5-, 6- or 7-membered heterocyclic ring;
R12, R16, R19 and R17 are as defined for R2, R6, R9 and R7 respectively;
R″ is a C3-12 alkylene group, which chain may be interrupted by one or more heteroatoms and/or aromatic rings that are optionally substituted; and X and X′ are independently selected from O, S and N(H); and wherein the antibody comprises: (i) HVR-H1 comprising the amino acid sequence of SEQ ID NO: 9, (ii) HVR-H2 comprising the amino acid sequence of SEQ ID NO: 10, (iii) HVR-H3 comprising the amino acid sequence of SEQ ID NO: 11, (iv) HVR-L1 comprising an amino acid sequence selected from SEQ ID NOs: 12 and 15 to 22, (v) HVR-L2 comprising the amino acid sequence of SEQ ID NO: 13, and (vi) HVR-L3 comprising the amino acid sequence of SEQ ID NO: 14.

75. The method of claim 74, wherein the CD22-positive cancer is selected from -lymphoma, non-Hogkins lymphoma (NHL), aggressive NHL, relapsed aggressive NHL, relapsed indolent NHL, refractory NHL, refractory indolent NHL, chronic lymphocytic leukemia (CLL), small lymphocytic lymphoma, leukemia, hairy cell leukemia (HCL), acute lymphocytic leukemia (ALL), Burkitt's lymphoma, and mantle cell lymphoma.

76. The method of claim 74 further comprising administering an additional therapeutic agent to the individual.

77. An in vitro method of inhibiting proliferation of a CD22-positive cell, the method comprising exposing the cell to the immunoconjugate of claim 46 under conditions permissive for binding of the immunoconjugate to CD22 on the surface of the cell, thereby inhibiting proliferation of the cell.


SEQUENCE LISTING (52 sequences)

SEQ ID NO: 1 - protein, 113 aa, Homo sapiens
EVQLVESGGGLVQPGGSLRLSCAASGFTFSSYAMSWVRQAPGKGLEWVSVISGDGGSTYYADSVKGRFTISRDNSKNTLYLQMNSLRAEDTAVYYCARGFDYWGQGTLVTVSS

SEQ ID NO: 2 - protein, 107 aa, Homo sapiens
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIK

SEQ ID NO: 3 - protein, 120 aa, Mus musculus
QVQLQQSGPELVKPGASVKISCKASGYEFSRSWMNWVKQRPGQGREWIGRIYPGDGDTNYSGKFKGKATLTADKSSSTAYMQLSSLTSVDSAVYFCARDGSSWDWYFDVWGAGTTVTVSS

SEQ ID NO: 4 - protein, 112 aa, Mus musculus
DILMTQTPLSLPVSLGDQASISCRSSQSIVHSNGNTFLEWYLQKPGQSPKLLIYKVSNRFSGVPDRFSGSGSGTDFTLKISRVEAEDLGVYYCFQGSQFPYTFGGGTKVEIK

SEQ ID NO: 5 - protein, 120 aa, Artificial sequence
  Synthetic
EVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSS

SEQ ID NO: 6 - protein, 112 aa, Artificial sequence
  Synthetic
DIQMTQSPSSLSASVGDRVTITCRSSQSIVHSNGNTFLEWYQQKPGKAPKLLIYKVSNRFSGVPSRFSGSGSGTDFTLTISSLQPEDFATYYCFQGSQFPYTFGQGTKVEIK

SEQ ID NO: 7 - protein, 120 aa, Artificial sequence; identical to SEQ ID NO: 5
  Synthetic

SEQ ID NO: 8 - protein, 112 aa, Artificial sequence
  Synthetic
DIQMTQSPSSLSASVGDRVTITCRSSQSIVHSVGNTFLEWYQQKPGKAPKLLIYKVSNRFSGVPSRFSGSGSGTDFTLTISSLQPEDFATYYCFQGSQFPYTFGQGTKVEIK

SEQ ID NO: 9 - protein, 10 aa, Artificial sequence
  Synthetic
GYEFSRSWMN

SEQ ID NO: 10 - protein, 18 aa, Artificial sequence
  Synthetic
GRIYPGDGDTNYSGKFKG

SEQ ID NO: 11 - protein, 11 aa, Artificial sequence
  Synthetic
DGSSWDWYFDV

SEQ ID NO: 12 - protein, 16 aa, Artificial sequence
  Synthetic
RSSQSIVHSNGNTFLE

SEQ ID NO: 13 - protein, 7 aa, Artificial sequence
  Synthetic
KVSNRFS

SEQ ID NO: 14 - protein, 9 aa, Artificial sequence
  Synthetic
FQGSQFPYT

SEQ ID NO: 15 - protein, 16 aa, Artificial sequence
  Synthetic
RSSQSIVHSVGNTFLE

SEQ ID NO: 16 - protein, 16 aa, Artificial sequence
  Synthetic
RSSQSIVHSAGNTFLE

SEQ ID NO: 17 - protein, 16 aa, Artificial sequence
  Synthetic
RSSQSIVHSQGNTFLE

SEQ ID NO: 18 - protein, 16 aa, Artificial sequence
  Synthetic
RSSQSIVHSSGNTFLE

SEQ ID NO: 19 - protein, 16 aa, Artificial sequence
  Synthetic
RSSQSIVHSDGNTFLE

SEQ ID NO: 20 - protein, 16 aa, Artificial sequence
  Synthetic
RSSQSIVHSIGNTFLE

SEQ ID NO: 21 - protein, 16 aa, Artificial sequence
  Synthetic
RSSQSIVHSNGITFLE

SEQ ID NO: 22 - protein, 16 aa, Artificial sequence
  Synthetic
RSSQSIVHSNGQTFLE

SEQ ID NO: 23 - protein, 219 aa, Artificial sequence
  Synthetic
DIQMTQSPSSLSASVGDRVTITCRSSQSIVHSVGNTFLEWYQQKPGKAPKLLIYKVSNRFSGVPSRFSGSGSGTDFTLTISSLQPEDFATYYCFQGSQFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 450 aa, Artificial sequence
  Synthetic
EVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 219 aa, Artificial sequence
  Synthetic
DIQMTQSPSSLSASVGDRVTITCRSSQSIVHSVGNTFLEWYQQKPGKAPKLLIYKVSNRFSGVPSRFSGSGSGTDFTLTISSLQPEDFATYYCFQGSQFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 26 - protein, 450 aa, Artificial sequence
  Synthetic
EVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 450 aa, Artificial sequence
  Synthetic
EVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 28 - protein, 847 aa, Homo sapiens
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACARCNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPHHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPRTCDDTVTYSALHKRQVGDYENVIPDFPEDEGIHYSELIQFGVGERPQAQENVDYVILKH

SEQ ID NO: 29 - protein, 828 aa, Homo sapiens
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACARCNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPHHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPRTCDDTVTYSALHKRQVGDYENVIPDFPEDEGIHYSELIQFGVGERPQAQENVDYVILKH

SEQ ID NO: 30 - protein, 670 aa, Homo sapiens
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 31 - protein, 651 aa, Homo sapiens
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 32 - protein, 10 aa, Artificial sequence
  Synthetic
GYTFSSYWIE

SEQ ID NO: 33 - protein, 18 aa, Artificial sequence
  Synthetic
GEILPGGGDTNYNEIFKG

SEQ ID NO: 34 - protein, 10 aa, Artificial sequence
  Synthetic
TRRVPIRLDY

SEQ ID NO: 35 - protein, 15 aa, Artificial sequence
  Synthetic
KASQSVDYEGDSFLN

SEQ ID NO: 36 - protein, 7 aa, Artificial sequence
  Synthetic
AASNLES

SEQ ID NO: 37 - protein, 9 aa, Artificial sequence
  Synthetic
QQSNEDPLT

SEQ ID NO: 38 - protein, 117 aa, Artificial sequence [sequence omitted]

SEQ ID NO: 39 - protein, 112 aa, Artificial sequence
  Synthetic
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKR

SEQ ID NO: 40 - protein, 446 aa, Artificial sequence
  Synthetic
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 218 aa, Artificial sequence
  Synthetic
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 218 aa, Artificial sequence
  Synthetic
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 43 - protein, 447 aa, Artificial sequence
  Synthetic
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 44 - protein, 446 aa, Artificial sequence
  Synthetic
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 25 aa, Artificial sequence [sequence omitted]

SEQ ID NO: 46 - protein, 13 aa, Artificial sequence
  Synthetic
WVRQAPGKGLEWV

SEQ ID NO: 47 - protein, 32 aa, Artificial sequence
  Synthetic
RFTISADTSKNTAYLQMNSLRAEDTAVYYCAR

SEQ ID NO: 48 - protein, 11 aa, Artificial sequence
  Synthetic
WGQGTLVTVSS

SEQ ID NO: 49 - protein, 23 aa, Artificial sequence
  Synthetic
DIQMTQSPSSLSASVGDRVTITC

SEQ ID NO: 50 - protein, 15 aa, Artificial sequence
  Synthetic
WYQQKPGKAPKLLIY

SEQ ID NO: 51 - protein, 32 aa, Artificial sequence
  Synthetic
GVPSRFSGSGSGTDFTLTISSLQPEDFATYYC

SEQ ID NO: 52 - protein, 10 aa, Artificial sequence
  Synthetic
FGQGTKVEIK